(19) United States
(12) Patent Application Publication
(10) Pub. No.: US 2026/0306407 A1
(43) Pub. Date: Oct. 8, 2026

(54) MODULAR MULTIPLATFORM SYSTEM FOR MRNA DRUG PRODUCTION

(71) Applicant: BioNTech SE, Mainz (DE)

(72) Inventors: Rainer Kröner, Kirchhain (DE); Semra Kati, Hamburg (DE); Florian Krämer, Andernach (DE); Jonas Hennekes, Mainz (DE)

(21) Appl. No.: 19/480,552

(22) PCT Filed: Jun. 28, 2024
(86) PCT No.: PCT/EP2024/068256

§ 371 Date: Oct. 31, 2025

Related U.S. Application Data
(60) Provisional application No. 63/511,175, filed on Jun. 29, 2023.
(60) Provisional application No. 63/588,670, filed on Oct. 6, 2023.
(60) Provisional application No. 63/614,026, filed on Dec. 22, 2023.

(51) Int. Cl. A61K 9/1277; A61K 9/51; A61K 39/00; A61L 2/022; A61L 2/22; A61L 103/10; B01D 15/32; B01D 61/14; B01D 69/02; B01D 71/16; B01J 19/00; B65B 3/00; B65B 7/28; B65B 55/10; B65B 65/00; F04B 43/12

(52) U.S. Cl. CPC A61K 9/1277; A61K 9/5123; A61K 9/5192; A61K 39/0011; A61K 39/001156; A61K 39/001163; A61K 39/00119; A61L 2/022; A61L 2/22; B01D 15/327; B01D 61/14; B01D 69/02; B01D 71/16; B01J 19/004; B01J 19/0066; B65B 3/003; B65B 7/2857; B65B 55/10; B65B 65/003; F04B 43/12; A61K 2039/53; A61K 2039/55555; A61K 2039/70; A61L 2103/10; B01D 2315/10; B01D 2325/02834; B01J 2219/0002

(57) ABSTRACT

A drug production facility includes: a first module comprising drug substance and drug product manufacturing equipment including equipment for producing RNA-based drug substance, lipid nanoparticle (LNP)-based drug products and lipoplex-based drug products; and a second module that includes fill and finish process equipment.

### CROSS-REFERENCE TO RELATED APPLICATIONS

This application claims priority to U.S. Provisional Patent Application No. 63/511,175, filed Jun. 29, 2023, the title of which is “MODULAR DRUG PRODUCTION SYSTEM”; U.S. Provisional Patent Application No. 63/588,670, filed Oct. 6, 2023, the title of which is “MODULAR MULTIPLATFORM SYSTEM FOR MRNA DRUG PRODUCTION”; and U.S. Provisional Patent Application No. 63/614,026, filed Dec. 22, 2023, the title of which is “MODULAR MULTIPLATFORM SYSTEM FOR MRNA DRUG PRODUCTION”, the content of each of which is incorporated herein by reference in its entirety.

### BACKGROUND

Nucleic acids represent an important therapeutic modality; lipid nanoparticle technologies have proven to be particularly useful for the delivery of nucleic acid therapeutics, specifically including RNA therapeutics (for example, mRNA therapeutics). The ability to deliver lipid nanoparticles, nucleic acids, and/or drug products resulting therefrom, (as well as other drug products) in a time-sensitive manner is often constrained by drug product (i.e., bulk drug product) manufacturing capacity, as well as the ability to rapidly adapt drug product manufacturing to current needs.

### SUMMARY

The present disclosure provides technologies relating to modular drug product manufacturing (i.e., bulk and individualized drug production) including mRNA vaccines and therapies, lipid nanoparticle (LNP) compositions, liposomes, lipoplexes, as well as other drug products and delivery modalities. Quickly producing and delivering therapies and/or treatments to patients often involves challenges relating to but not limited to manufacturing capacity, manufacturing flexibility, the overall time required to produce and deliver a drug product to a patient, as well as other considerations. Often, local requirements relating to certification and approval of drugs categorize drug products differently depending on if they are produced within a country or imported from another country. For example, different regulatory approval hurdles may exist for drugs that are produced within a country than those that exist for drugs that are imported.

In one aspect, the present disclosure is directed to a drug production facility that includes: a first module comprising drug substance and drug product (for example, bulk drug product in some embodiments) manufacturing equipment comprising equipment for producing RNA-based drug substance, lipid nanoparticle (LNP)-based drug products and lipoplex-based drug product; and a second module comprising fill and finish process equipment.

In some embodiments, the first module contains: an RNA-based drug substance; and an LNP-based drug product and/or a lipoplex-based drug product.

In some embodiments, each of the first module and the second module comprises a Grade C Clean Room area of from about 80 square meters to about 120 square meters.

In some embodiments, the second module comprises a first machine for debagging, a second machine for denesting, a third machine for filling and a fourth machine for capping and crimping.

In some embodiments, the second module further comprises at least one conduit supplying laminar Grade A air to an air bathing area, the air bathing area being located in a vicinity of the first machine for debagging is located.

In some embodiments, the second module further comprises: a first mixing unit for pooling multiple drug products, each drug product of the multiple drug products comprising different RNA, the multiple drug products being used in a single, multivalent vaccine; and a second mixing unit for performing sterile filtration (i.e., a final sterile filtration). In some embodiments, the contents are mixed and cooled within the second mixing unit, and the weight of the contents is also measured within the second mixing unit.

In some embodiments, the second module further comprises at least one of: lyophilization equipment, an autocalve, washing machinery (i.e., glass and/or metallic component washing machinery), sterilizing misting equipment, glove box testing equipment, and a wall pass through.

In some embodiments, the second module further comprises at least one conduit supplying nitrogen to the filling machine.

In some embodiments, the second module comprises sterilizing misting equipment, and the sterilizing misting equipment is configured to provides a sterilizing mist comprising ionized hydrogen peroxide.

In some embodiments, the first module comprises a chromatograph (for example, a cellulose chromatograph, a chromatograph that removes oligo (dT) primers, and/or a chromatograph that performs hydrophobic interaction chromatography (HIC)).

In some embodiments, the first module comprises multiple sensors comprising: at least one pressure sensor; at least one combined temperature and humidity sensor at least one hydrogen peroxid sensor; at least one ethanol sensor; and at least one oxygen sensor.

In some embodiments, the first module contains a lipoplex-based drug product comprising lipoplex particles that comprise a phospholipid bilayer structure that encapsulates RNA molecules of the RNA-based drug substance.

In some embodiments, the first module contains a lipoplex-based drug product comprising lipoplex particles that comprise a cationic lipid or an ionizable aminolipid in a 2:1 molar ratio with a helper lipid.

In some embodiments, the first module contains a lipoplex-based drug product comprising lipoplex particles that comprise a particle size in a range from about 250 nm to about 700 nm.

In some embodiments, the first module contains a lipoplex-based drug product encapsulating the RNA-based drug substance, wherein RNA of the RNA-based drug substance encodes at least one of a carcinoma antigen, a melanoma-associated antigen, a tyrosinase antigen, and a transmembrane phosphatase with tensin homology (TPTE) antigen.

In another aspect, the present disclosure is directed to: a method of selectively producing a filled container containing an RNA-based drug product at a production facility comprising a first module and a second module, the method comprising: producing an RNA-based drug substance within the first module, wherein producing drug substance comprises: performing in vitro transcription (IVT); performing at least one tangential flow filtration (TFF) step and/or at least one hydrophobic interaction chromatography (HIC) step; and performing at least one filtration step following TFF; producing, within the first module, a lipid-based drug product that comprises the RNA-based drug substance, the lipid-based drug product comprising at least one of a lipoplex particle and a lipid nanoparticle (LNP); and performing a filling process, within the second module, comprising filling at least one fluid container with the lipid-based drug product.

In some embodiments, producing an RNA-based drug substance comprises performing chromatography prior to a TFF step of the at least one tangential flow filtration (TFF) step.

In some embodiments, performing a filling process comprises: bathing a bag holding the RNA-based drug product in laminar Grade A air while a debagging process occurs to remove the bag; and filling the container with the RNA-based drug product using an automated filling machine.

In some embodiments, the bag holding the RNA-based drug product is either a) holding a bag that directly holds the RNA-based drug product, or b) is itself held by another bag (i.e., the RNA-based drug product is double bagged).

In some embodiments in which the RNA-based drug product is double bagged, the outer bag is removed in the area that is bathed in laminar Grade A air, and the inner bag is removed in a debagging machine.

In some embodiments, the lipid-based drug product comprises a lipid nanoparticle (LNP), and producing a lipid-based drug product comprises: performing an LNP formation step comprising impingement jet mixing; performing a tangential flow filtration (TFF) step following the LNP formation step; and performing at least one formulation step following the TFF step.

In some embodiments, the lipid-based drug product comprises a lipoplex particle, and producing a lipid-based drug product comprises: conditioning RNA from the RNA-based drug substance; performing sterile filtration on the conditioned RNA; and performing a lipoplexation step to produce the lipoplex particle.

The present embodiments include drug production facilities that are configured to accommodate the production of different types and quantities of drugs including mRNA-based vaccines delivered via lipid nanoparticle and/or lipoplex delivery systems. In one embodiment, the present disclosure is directed to a portable system for producing a formulation comprising an mRNA-based product including: a first sub-system including a drug substance formulation module, the first sub-system including: transcription equipment for forming an RNA solution via in vitro transcription; and a second sub-system operatively downstream of the first sub system comprising multiple drug product formation modules, the second sub-system including: an LNP formation module for producing an RNA-LNP preparation from the RNA solution; a lipoplex formation module for producing an RNA lipoplex preparation from the RNA solution; and a buffer preparation module (or dispensing room, for lipid and buffer dispensing). The portable system may be contained within an area no larger than about 75 square meters. In some embodiments, the portable system may be contained within an area no larger than about 88 square meters including about 75 square meters for the production area and about 13 square meters for the dispensing area/preparation area.

In some embodiments, the lipoplex formation module comprises continuous automated lipoplex formation (CALF) equipment.

In some embodiments, the lipoplex formation module comprises at least one of a mixing unit, a biowelder, a biosealer, a refrigerator, a sonicator, and a bag tester.

In some embodiments, the second sub-system further comprises a shared use area.

In some embodiments, the shared use area comprises at least one of a charging station, a mobile HMI, a magnetic stirrer, and a filter holder.

In some embodiments, the buffer preparation module comprises at least one of a magnetic stirrer, a buffer mixing unit, a pH sensor, a scale, a balance, a conductivity sensor, and/or optionally a temperature control unit (TCU) for heating/cooling.

In another aspect, the present embodiments are directed to a facility that includes the system described herein and a portable fill and finish module.

In some embodiments, the fill and finish module comprises lyophilization equipment.

In some embodiments, the fill and finish module comprises autoclave equipment (e.g., one or more autoclaves).

In some embodiments, the fill and finish module comprises machine-washing equipment (i.e., glass, metallic, ceramic, polymer, etc. washing machinery).

In another aspect, the present embodiments are direct to a method for making liposomes in a modular and/or portable production facility, the method including: adjusting a lipid concentraction within a first solution; adding the lipid concentration to, and mixing it with, ethanol; sterilizing the mixture via filtration; injecting the filtered mixture into a spinner flask; stirring the filtered mixture within the spinner flask; continuing injection of the mixture into the spinner flask until a desired lipid concentration is reached; filtering the mixture, thereby forming filtered liposomes; and diluting the filtered liposomes.

In some embodiments, the method includes storing the diluted liposomes in bags.

In some embodiments, filtration comprises filtering with a cellulose acetate filter.

In some embodiments, filtration comprises filtering with a pore size in a range from about 0.1 micrometers to about 0.5 micrometers.

In some embodiments, the method includes: prior to adjusting the lipid concentraction within a first solution, providing filtration equipment, injection equipment, and at least one spinner flask to the modular and/or portable production facility described herein.

In another aspect, the present embodiments are directed to a method for making lipoplexes in a modular and/or portable production facility, the method including: providing an RNA solution and a liposome solution to the modular and/or portable production facility; adjusting a concentration of RNA in the RNA solution to a concentration of liposomes in the liposome solution; adding NaCl to the RNA solution for condensation of the RNA; and mixing the RNA solution and the liposome solution, thereby forming a lipoplex solution.

In some embodiments, adjusting a concentration of RNA in the RNA solution comprises mixing positively charged liposomes in the liposome solution with RNA molecules in the RNA solution such that the positively charged lipids and RNA are present at a charge ratio of 1.3 to 2 to facilitate delivery of the resulting lipoplexes in the lipoplex solution to a target location.

In some embodiments, the liposome solution comprises cationic lipids.

In some embodiments, the liposome solution comprises at least one of a cationic lipid, an ionizable aminolipid, and a helper lipid.

In some embodiments, mixing the RNA solution and the liposome solution includes: transferring both the RNA solution and the liposome solution into large-volume syringes; mounting both large-volume syringes onto a single syringe pump; and simultaneously driving the pistons of each of the respective large-volume syringes, thereby enabling lipoplex formation.

In some embodiments, the method further includes: adding a cryoprotectant solution to the lipoplex solution; and adjusting a concentration of the lipoplex solution to arrive at a final concentration.

In some embodiments, the method further includes filling containers (for example, glass vials or bottles) with the lipoplex solution; freezing the filled containers, thereby forming a lipoplex concentrate; and storing the frozen and filled containers in a temperature-controlled environment.

In some embodiments, adjusting a concentration of RNA in the RNA solution includes adjusting the RNA concentration in the RNA solution to allow for mixing of identical volumes of RNA and liposomes.

In some embodiments, the method includes providing a pre-sterilized single use fluid path to the modular and/or portable production facility prior to providing the RNA solution and the liposome solution to the modular and/or portable production facility, to allow for safe aseptic handling of the materials (e.g., solutions).

In some embodiments, the method includes providing in-line cleaning systems to the modular and/or portable production facility prior to providing the RNA solution and the liposome solution to the modular and/or portable production facility, to allow for aseptic conditions within at least one multi-use fluid path.

In some embodiments, the single syringe pump comprises a single perfusor pump.

In some embodiments, the method includes providing continuous automated lipoplex formation (CALF) equipment to the modular and/or portable production facility prior to mixing the RNA solution and the liposome solution, wherein mixing the RNA solution and the liposome solution comprises mixing the RNA solution and the liposome solution using the continuous automated lipoplex formation (CALF) equipment.

In some embodiments, mixing the RNA solution and the liposome solution includes one or more ethanol injection techniques.

In some embodiments, mixing the RNA solution and the liposome solution includes one or more sonication steps.

In some embodiments, mixing the RNA solution and the liposome solution comprises mixing using at least one of a Y-type mixing element and a T-type mixing element, and each of the Y-type mixing element and the T-type mixing element includes an inner diameter in a range from about 1.0 mm and to about 50.0 mm. In some embodiments, each of the Y-type mixing element and the T-type mixing element includes an inner diameter in a range from about 1.0 mm and to about 10.0 mm.

In some embodiments, the method includes providing a mixing unit, a biowelder, a biosealer, a refrigerator, a sonicator, and/or a bag tester to the modular and/or portable production facility prior to mixing the RNA solution and the liposome solution.

In another aspect, the present embodiments are directed to a portable system for producing a formulation comprising an mRNA-based product, the system including: a first sub-system comprising a drug substance formulation module, the first sub-system including: equipment for forming an RNA solution; and a second sub-system including multiple drug product formation modules, the second sub-system comprising: an LNP formation module for producing an RNA-LNP preparation from the RNA solution and a lipid solution; and a lipoplex formation module for producing an RNA-lipoplex preparation from the RNA solution and a liposome solution. The portable system is configured to selectively produce each of the RNA-LNP preparation and the RNA-lipoplex preparation using the RNA solution.

In another aspect, the present embodiments are directed to a method for making RNA lipid nanoparticles (LNP) in a modular production facility, the method including: providing an acidified aqueous phase, an RNA solution, and a lipid solution to the modular production facility; mixing the acidified aqueous phase with the lipid solution, thereby forming a mixture; performing at least one of filtration, bioburden reduction, freezing, and dilution on the mixture, thereby forming preformed LNPs; and mixing the RNA solution and the preformed LNPs, thereby forming the RNA lipid nanoparticles.

In some embodiments, the system described herein includes at least one in-process monitoring and/or control sensor.

In some embodiments, the in-process monitoring and/or control sensor includes a sensor for measuring bacteria count and/or a sensor for detecting at least one visible property or characteristic of a liquid.

In some embodiments, the system includes a sensor for measuring bacteria count includes at least one of a spectrophotometer, a dip tester, and an optical sensor configured to measure an optical density within a specific wavelength spectrum.

In some embodiments, the system includes a sensor for detecting at least one visible property or characteristic of a liquid (for example, a vortex).

In some embodiments, the system includes a sensor for detecting at least one visible property or characteristic of a liquid (for example, the existence or presence of at least one subvisible particle (SVP)).

In some embodiments, the cryoprotectant used in the systems and/or methods described herein includes: a sucrose buffer in a weight percent range from about 13% to about 18%; a HEPES buffer at a concentration of about 3.8 mM to about 4.6 mM; an EDTA butter at a concentration of about 0.8 mM to about 1.6 mM; and a pH of about 6.3 to about 7.1.

In another aspect, the present embodiments are directed to a modular drug production facility (or module) comprising a combined process flow capable of producing both lipid nanoparticle (LNPs) and lipoplexes, the facility (or module) including: an LNP formation module; a lipoplex formation module; a lipid supply; and a drug substance/RNA formation module. In some embodiments, each of the LNP formation module and the lipoplex formation module are fluidly coupled downstream of both the lipid supply and the drug substance/RNA formation module.

In some embodiments, the facility and/or system described herein includes a buffer exchange/purification module fluidly coupled downstream of both the lipid supply and the drug substance/RNA formation module.

In another aspect, the present embodiments are directed to an RNA mixture including: from about 42% to about 54% RNA solution by volume; from about 8% to about 28% elution buffer by volume; and from about 30% to about 38% dilution buffer by volume.

In some embodiments, the elution buffer includes a chelating agent.

In some embodiments, the elution buffer includes at least one of an 18 mM HEPES buffer at a pH of 7 and an 18 mM ETDA buffer at a pH of 7.

In some embodiments, the dilution buffer includes ammonium sulphate.

In some embodiments, the RNA mixture includes drug substance.

In another aspect, the present embodiments are directed to a process of making lipid nanoparticles (LNP) including impingement jet mixing the RNA solution as described herein, with an ethanol solution that includes lipids.

In another aspect, the present embodiments are directed to a bioburden reduction module that includes the RNA mixture as described herein, disposed within a filter that includes a 0.2 μm pore diameter.

In some embodiments, the RNA mixture includes mRNA, and the module includes an mRNA mass to filter surface area ratio in a range from about 1.0 mg/cm\hat{ }2 to about 2.0 mg/cm\hat{ }2.

In some embodiments, the liposome solution used in the methods and/or systems described herein includes: about 70% by weight phospholipon 100H loaded with sodium hyaluronic acid; about 20% by weight cholesterol; and about 10% by weight stearylamine.

In another aspect, the present embodiments are directed to a mixing unit that includes: multiple fluid inlets including a first fluid inlet and a second fluid inlet, the first fluid inlet including a first fluid flowing therethrough, the second fluid inlet including a second fluid flowing therethrough. In some embodiments, the second fluid includes the RNA mixture as described herein.

In some embodiments, the second fluid inlet further includes an ethanol solution including lipids.

In some embodiments, the CALF equipment described herein includes a low-pulsation peristaltic pump.

In some embodiments, the low-pulsation peristaltic pump includes from about 2 to about 4 rollers.

In some embodiments, the low-pulsation peristaltic pump includes at least one flow rate sensor in a feedback-loop configuration for online-control and real-time adjustment of a fluid flow rate, the fluid including at least one of a liposome solution and a lipoplex solution.

In some embodiments, the low-pulsation peristaltic pump includes two or more channels to reduce pulsations.

In some embodiments, the rollers are angularly offset from one another around an inner circumference of the low-pulsation peristaltic pump such that pulsations are minimized and/or eliminated.

In some embodiments: the low-pulsation peristaltic pump includes a dual head, the low-pulsation peristaltic pump operates at a flow rate in a range from about 1 mL/min to about 500 mL/min, and the low-pulsation peristaltic pump operates at a delta pressure range (i.e., the pressure difference between a pump discharge and a pump suction) in a range from about 0.05 bar to about 1 bar.

In some embodiments, the CALF equipment includes a rocker mixer to aid in lipoplexation.

In some embodiments, the facility and/or system described herein includes a liposome formation module disposed fluidly downstream of the lipid supply and fluidly upstream of the lipoplex formation module.

In some embodiments, the drug substance/RNA formation module produces an RNA solution.

In another aspect, the present embodiments are directed to a method of forming an RNA-lipid solution including: providing the facility as described herein; and selectively flowing lipids and RNA solution to the LNP formation module and/or the lipoplex formation module, thereby forming the RNA-lipid solution.

In some embodiments, selectively flowing lipids to the lipoplex formation module includes: flowing lipids to the liposome formation module; and flowing liposomes from the liposome formation module to the lipoplex formation module.

The present embodiments include a modular drug production system that may be shipped anywhere in the world that standard shipping (i.e., “overseas”) containers are able to be shipped. The modular drug production system of the present embodiments may be particularly useful for producing RNA-LNP drug products, but may also be useful for any number of other types of drug products. The modular drug production system of the present embodiments enables drugs to be produced within a country and/or localized region, thereby allowing for drug products that address localized outbreaks and viral strains to be manufactured, in the exact location in which they are needed. In addition, the modular drug production system of the present embodiments allows for an increase in the overall drug manufacturing capacity, and offloads the demand and/or pressure on centralized large scale drug manufacturing facilities to undertake smaller-scale production runs (i.e., to address regional and/or localized viral strains, for use in clinical trials, for commercial production of small batch therapies (for example, drug products for treating orphan diseases), etc.) to the possible detriment of large scale production (which is primarily focused on helping the greatest number people in the most efficient manner).

In another aspect, the present disclosed embodiments are directed to a biopharmaceutical manufacturing facility comprising at least one biopharmaceutical production unit, said biopharmaceutical production unit comprising two or more modularly arranged containment units containing one or more apparatus adaptable for (1) receipt and processing of active ingredient or active ingredient precursor, and pharmaceutically acceptable carrier or excipient, and resultant biopharmaceutical product production (2) biopharmaceutical product quality control, and (3) biopharmaceutical product filling and finishing, said two or more modularly arranged containment units being positioned and optionally interconnected for the inter-unit transfer of unfinished biopharmaceutical product and delivery of finished biopharmaceutical product.

In some embodiments, the facility comprises between one and ten production units (i.e., containers or modules), each of said production units comprising: (a) a first containment unit containing one or more apparatus adaptable for receipt and processing of active ingredient or active ingredient precursor, and pharmaceutically acceptable carrier or excipient, and resultant biopharmaceutical product production; (b) a second containment unit in communication with the first containment unit for the receipt of biopharmaceutical product and containing one or more apparatus adaptable for biopharmaceutical product quality control, and (3) a third containment unit in communication with the second containment unit for the receipt of quality control-evaluated biopharmaceutical product and containing one or more apparatus adaptable for filling and finishing of quality control-evaluated biopharmaceutical product and delivery of finished biopharmaceutical product.

In some embodiments, at least two of the modularly arranged containment units are horizontally arranged.

In some embodiments, the manufacturing facility is at least partially enclosed in a housing. In some embodiments, both the active ingredient and active ingredient precursor are each nucleotides, processing includes encapsulation of a processed nucleotide within a lipid nanoparticle and the resultant biopharmaceutical product is a nucleotide vaccine. In some embodiments, the active ingredient is mRNA.

In some embodiments, at least one of the containment units is an ISO container. In some embodiments, the one or more apparatus adaptable for receipt and processing of active ingredient or active ingredient precursor, and pharmaceutically acceptable carrier or excipient, and resultant biopharmaceutical product production include a bioreactor adaptable for receipt of DNA and the in vitro transcription of said DNA into mRNA. In some embodiments, the one or more apparatus adaptable for receipt and processing of active ingredient or active ingredient precursor, and pharmaceutically acceptable carrier or excipient, and resultant biopharmaceutical product production include purification apparatus configured for the removal of DNA impurities from the mRNA.

In some embodiments, the biopharmaceutical manufacturing facility produces from about 1 million to about 50 million doses of finished mRNA vaccine per year, or about 3,000 to about 170,000 doses of finished vaccine per day. In some embodiments, the apparatus adaptable for: (1) receipt and processing of active ingredient or active ingredient precursor, and pharmaceutically acceptable carrier or excipient, and resultant biopharmaceutical product production (2) biopharmaceutical product quality control, and (3) filling and finishing of quality control-evaluated biopharmaceutical product are in electronic communication with process control systems for remote operation. In some embodiments, the electronic communication is wireless; process control is at least partially automated; process control systems are controlled by one or more computers; and/or the one or more computers are remote from the facility.

In another aspect, the present disclosed embodiments are directed to a portable system for producing a formulation comprising lipid nanoparticle (LNP)-encapsulated RNA, the system comprising: a first sub-system comprising multiple drug substance formulation modules, the first sub-system comprising: a transcription module for forming an RNA solution via in vitro transcription (e.g., of a DNA template); and a second sub-system operatively downstream of the first sub-system comprising multiple drug product formation modules, the second sub-system comprising: an LNP formulation module for producing a first RNA-LNP preparation from the RNA solution, wherein each of the transcription module and the LNP formulation module is contained (or shipped to a site) within a separate standard shipping container.

In some embodiments, each separate standard shipping container comprises a width of about 8 ft (2.43 m), a height of about 8.5 ft (2.59 m) and a length from about 20 ft (6.06 m) to about 40 ft (12.12 m).

In some embodiments, the LNP formulation module comprises at least one impingement jet mixing unit. In some embodiments, the second sub-system further comprises a purification module disposed operatively downstream of the LNP formulation module, the purification module comprising at least one tangential flow filtration (TFF) unit, wherein the purification module is disposed within a separate standard shipping container. In some embodiments, wherein each of the first sub-system and the second sub-system comprises a bioburden reduction module disposed within a separate standard shipping container, each bioburden reduction module comprising a filtration unit comprising at least one filter with a pore size from about 0.05 μm to about 0.35 μm.

In another aspect, the present disclosed embodiments are directed to a portable system for producing a formulation comprising lipid nanoparticle (LNP)-encapsulated RNA, the system comprising: a first module comprising a transcription module for forming an RNA solution via in vitro transcription; a second module comprising a first purification module comprising a first tangential flow filtration (TFF) unit, the second module operatively downstream of the first module and receiving the RNA solution therefrom; a third module comprising a first bioburden reduction module, the third module operatively downstream of the second module; a fourth module comprising an LNP formulation module for producing a first RNA-LNP preparation from the RNA solution, the fourth module operatively downstream from the third module; a fifth module comprising a second purification module comprising a second tangential flow filtration (TFF) unit, the fifth module operatively downstream of the fourth module and receiving the first RNA-LNP preparation therefrom; and a sixth module comprising a second bioburden reduction module, the sixth module operatively downstream of the fifth module, wherein each of the first through sixth modules are disposed (and/or transported to a production site) within a separate standard shipping container.

In another aspect, the present disclosed embodiments are directed to a drug production system comprising: a drug substance module for producing at least one drug substance; a drug product module for producing a drug product at least partially from the at least one drug substance; wherein each of the drug substance module and the drug product module is disposed entirely in one or more portable shipping containers, and wherein the drug product is used to treat and/or vaccinate a patient within 1, 2, 4, 8, 12, 24, 48, and/or 72 hours of being produced.

In another aspect, the present disclosed embodiments are directed to a portable LNP formulation system for producing a first RNA-LNP preparation comprising: an impingement jet mixing unit, a first fluid conduit for delivering an RNA solution to the impingement jet mixing unit, the first fluid conduit fluidly connecting the impingement jet mixing unit to an RNA solution source external to the system; a second fluid conduit for delivering a lipid solution to the impingement jet mixing unit, the second fluid conduit fluidly connecting the impingement jet mixing unit to a lipid solution source external to the system; and a third fluid conduit for delivering the first RNA-LNP preparation to a downstream module external to the system, wherein the system is disposed (and/or transported to a production site) within a single standard shipping container.

In another aspect, the present disclosed embodiments are directed to a portable LNP formulation system for producing a first RNA-LNP preparation comprising: an impingement jet mixing unit; and a tangential flow filtration (TFF) unit coupled fluidly downstream of the impingement jet mixing unit, the TFF unit for performing at least one diafiltration step and at least one ultrafiltration step, wherein the system is disposed (and/or transported to a production site) within a single standard shipping container.

In some embodiments, the system includes a bioburden reduction unit coupled fluidly downstream of the TFF unit and contained within the standard shipping container.

In some embodiments, the system includes a first fluid conduit for delivering an RNA solution to the impingement jet mixing unit, the first fluid conduit fluidly connecting the impingement jet mixing unit to an RNA solution source external to the system; a second fluid conduit for delivering a lipid solution to the impingement jet mixing unit, the second fluid conduit fluidly connecting the impingement jet mixing unit to a lipid solution source external to the system; and a third fluid conduit for delivering a first RNA-LNP preparation to the TFF unit.

In another aspect, the present disclosed embodiments are directed to a drug production system comprising: a drug substance module for producing at least one drug substance; a drug product module for producing a drug product comprising the at least one drug substance; wherein each of the drug substance module and the drug product module are disposed entirely in one or more portable shipping containers.

In some embodiments, each of the drug substance module and the drug product module are disposed within at least three (3) portable shipping containers. In some embodiments, the drug product comprises at least one lipid nanoparticle (LNP). In some embodiments, the system includes at least one fill and finish module for disposing the drug product into at least one container.

In some embodiments, the combined power requirement of the drug substance module and the drug product module is in a range from about 200 kW to about 400 kW with an uninterrupted power requirement in a range from about 50 kW to about 100 kW.

In some embodiments, the combined footprint of the drug substance module and the drug product module encompasses an area of from about 500 square meters to about 1000 square meters.

In some embodiments, each of the drug substance module and the drug product module includes at least one airlock through materials and/or personnel must pass when entering an operations area within the respective drug substance module and drug product module.

In some embodiments, the system includes a quality control module including a PCR lab, an RNA/DNA lab, an environmental monitoring console, an HPLC lab, a cell culture lab, a general procedure lab, freezer monitoring equipment, a bioburden lab, a quality control storage area, a washing area, an endotoxin lab, and/or a gowning area.

The present disclosure provides technologies for improving LNP manufacturing, transport and/or storage. Those skilled in the art, reading the present disclosure, will appreciate the significance and applicable breadth of its teachings.

Among other things, those skilled in the art will appreciate the increasing significance of nucleic acid therapeutics (e.g., oligonucleotide therapeutics, as well as longer DNA and/or RNA therapeutics), including the transformative impact of RNA vaccines during the COVID19 pandemic.

Those skilled in the art will further appreciate the importance of delivery technologies, and particularly of LNP delivery technologies, to the success of nucleic acid therapeutics, specifically including therapeutic RNAs e.g., therapeutic mRNAs.

In some embodiments, provided technologies are useful for manufacturing pharmaceutical-grade RNA therapeutics). In some embodiments, provided technologies may be particularly useful for large scale manufacturing of RNA therapeutics, e.g., of pharmaceutical-grade RNA therapeutics.

Among other things, in some embodiments, the present disclosure identifies the source of one or more challenges that can be associated with manufacturing and/or maintaining certain LNP compositions, specifically including RNA-LNP compositions. Among other things, the present disclosure provides technologies that facilitate consistent manufacturing, for example, satisfying pre-determined in-process controls, and/or lot release specifications (e.g., high purity, integrity, potency, etc.). In some embodiments, the present disclosure provides robust manufacturing technologies for LNP (e.g., RNA-LNP) compositions, including technologies that can be performed at scale, while maintaining particular product attributes, such as high purity, integrity, stability (e.g., to transportation and/or storage) etc. In some embodiments, relevant product attributes may be or include, for example, colloidal stability, particle size (and/or size distribution), LNP topology, amenability to further processing and/or formulation, effectiveness of delivery of encapsulated material from administered compositions, etc.

In some embodiments, technologies described herein can be utilized in different manufacturing scales. In some embodiments, technologies described herein can be utilized in parallel to further improve throughput capacity.

In some embodiments, technologies provided herein can utilize nucleic acid (e.g., RNA) manufactured in batch sizes within a range of about 0.01 g to about 500 g, about 0.01 g to about 10 g, about 1 g to about 10 g, about 10 g to about 500 g, about 10 g to about 300 g, about 10 g to about 200 g or about 30 g to about 60 g.

Among other things, in some embodiments, the present disclosure provides methods for characterizing LNP products (e.g., nucleic acid-LNP products such as RNA-LNP products) manufactured that are suitable for use in pharmaceutical products.

Technologies described herein can be useful for manufacturing LNP compositions (e.g., of LNPs encapsulating nucleic acid, and in particular RNA such as, e.g., mRNA). In some embodiments, technologies described here can be useful for manufacturing LNP compositions (e.g., nucleic acid-LNPs, e.g., RNA-LNPs) for treatment and/or prevention of a disease, disorder, or condition (e.g., cancer, infectious diseases, diseases associates with protein deficiency, etc.).

In some embodiments, technologies described herein can be useful for manufacturing LNP compositions that comprise or deliver a nucleic acid encoding a polypeptide. In some embodiments, technologies described herein can be useful for manufacturing LNP for inducing an immune response to an antigen (e.g., an antigen encoded by a nucleic acid that is included in or delivered by an LNP composition).

In some embodiments, the present disclosure is directed to a system that includes at least one DNA sequencer for sequencing at least one local strain of a disease.

In some embodiments, the system includes at least one DNA synthesizer for creating at least one custom DNA molecule.

In some embodiments, the system includes at least one computing system for performing at least one of the following tasks: uploading sequence information describing at least one local strain of a disease to a public database, downloading sequence information describing the at least one local strain of a disease from the public database, downloading DNA synthesis data to be used for making a vaccine that targets the at least one local strain of a disease from the public database, and computing, based on the sequence information describing at least one local strain of a disease, a target strain upon which DNA synthesis data is based.

In another aspect, the present disclosed is directed to a method of producing a vaccine to treat a local strain of a disease, the method comprising: filtering genomic data for the disease by at least one location, thereby producing localized data; determining a target strain from the localized data; sending DNA synthesis instructions to a site within, or proximate to, the at least one location; producing, within, or proximate to, the at least one location, the vaccine to treat the local strain based on the DNA synthesis instructions.

In some embodiments, the disease is SARS-CoV-2.

In some embodiments, the method includes administering the vaccine and distributing the vaccine within, or proximate to, the at least one location.

In some embodiments, the method includes accessing a publicly available database that houses the genomic data to be filtered.

In some embodiments, the method includes: sequencing, within, or proximate to, the at least one location, a sample of the local strain of the disease, thereby producing local strain sequence data; and uploading the local strain sequence data to a public ally available database that houses the genomic data to be filtered.

In some embodiments, the method includes filtering the genomic data based on at least one of a range of dates and a lookback period. The range of dates and/or lookback period corresponds to a timeframe during which a localized outbreak of the disease occurred.

In some embodiments, the method includes assessing deviations between the localized data and a baseline variant of the disease.

In some embodiments, the method includes comparing the deviations for one or more subsets within the localized data.

In some embodiments, the method includes assessing a level of commonality of the deviations for the one or more subsets within the localized data.

In some embodiments, determining a target strain from the localized data includes determining a target strain based at least partially on the deviations between the localized data and the baseline variant of the disease.

In some embodiments, technologies described herein can be useful for manufacturing RNA-LNP compositions for treatment and/or prevention of coronavirus infection, e.g., SARS-CoV-2 infection, as described in Walsh et al. “RNA-based COVID-19 vaccine BNT162b2 selected for a pivotal efficacy study” medRxiv preprint (2020), which is online accessible at: https://doi.org/10.1101/2020.08.17.20176651; and Milligan et al. “Phase I/II study of COVID-19 RNA vaccine BNT162b1 in adults” Nature (2020 August), which is online accessible at: https://doi.org/10.1038/s41586-020-2639-4, the contents of each of which are herein incorporated by reference in their entirety.

### BRIEF DESCRIPTION OF THE DRAWING

FIG. 1 depicts an exemplary modular drug production system, according to aspects of the present disclosure.

FIG. 2 depicts an overview of an exemplary drug product manufacturing enterprise and/or process, according to aspects of the present disclosure.

FIG. 3 depicts an overview of an exemplary drug product manufacturing site and/or process, according to aspects of the present disclosure.

FIG. 4 depicts an overview of an exemplary drug product manufacturing site, according to aspects of the present disclosure.

FIG. 5 depicts an overview of an exemplary drug product manufacturing site and/or module, according to aspects of the present disclosure.

FIG. 6 depicts an overview of an exemplary drug product manufacturing site and/or module, according to aspects of the present disclosure.

FIG. 7 depicts an overview of an exemplary drug product quality control site and/or module, according to aspects of the present disclosure.

FIG. 8 depicts an overview of an exemplary drug product warehouse site and/or module, according to aspects of the present disclosure.

FIG. 9 depicts an overview of an exemplary drug product manufacturing site, according to aspects of the present disclosure.

FIG. 10 depicts an overview of an exemplary drug product manufacturing site, according to aspects of the present disclosure.

FIG. 11 depicts an overview of exemplary manufacturing process for a pharmaceutical-grade composition comprising RNA, according to aspects of the present disclosure.

FIG. 12 illustrates an overview of exemplary DNA template manufacture process via a PCR-based process, according to aspects of the present disclosure.

FIG. 13 illustrates an exemplary process for manufacturing LNP compositions, according to aspects of the present disclosure.

FIG. 14 depicts an overview of an exemplary drug product manufacturing site and/or module, according to aspects of the present disclosure.

FIG. 15 depicts an overview of an exemplary drug product manufacturing site and/or module, according to aspects of the present disclosure.

FIG. 16 depicts an overview of an exemplary drug product manufacturing site and/or module, according to aspects of the present disclosure.

FIG. 17 illustrates a process for making vaccines, according to aspects of the present disclosure.

FIG. 18 illustrates a process for making vaccines, according to aspects of the present disclosure.

FIG. 19 depicts an overview of an exemplary drug product manufacturing enterprise and/or process, according to aspects of the present disclosure.

FIG. 20 depicts an overview of an exemplary drug product manufacturing enterprise and/or process, according to aspects of the present disclosure.

FIG. 21 depicts an overview of an exemplary drug product manufacturing enterprise and/or process, according to aspects of the present disclosure.

FIG. 22 illustrates a process for making liposomes, according to aspects of the present disclosure.

FIG. 23 illustrates a process for making lipoplexes, according to aspects of the present disclosure.

FIG. 24 depicts an overview of an exemplary drug product manufacturing site and/or module, according to aspects of the present disclosure.

FIG. 25 depicts an overview of an exemplary fill and finish module, according to aspects of the present disclosure.

FIG. 26 depicts an overview of a combined lipid nanoparticle (LNP) and lipoplex (LPX) process flow, according to aspects of the present disclosure.

FIG. 27 depicts an overview of an exemplary drug product manufacturing site and/or module, according to aspects of the present disclosure.

FIG. 28A depicts an overview of an exemplary fill and finish module, according to aspects of the present disclosure.

FIG. 28B depicts an overview of an exemplary fill and finish module, according to aspects of the present disclosure.

FIG. 28C depicts an overview of an exemplary fill and finish module, according to aspects of the present disclosure.

FIG. 29 depicts an overview of an exemplary drug substance workflow, according to aspects of the present disclosure.

FIG. 30 depicts an overview of an exemplary drug substance workflow, according to aspects of the present disclosure.

FIG. 31 depicts an overview of an exemplary drug substance workflow, according to aspects of the present disclosure.

FIG. 32 depicts an overview of an exemplary drug product workflow, according to aspects of the present disclosure.

FIG. 33 depicts an overview of an exemplary drug product workflow, according to aspects of the present disclosure.

FIG. 34 depicts an overview of an exemplary drug product workflow, according to aspects of the present disclosure.

FIG. 35 depicts an overview of an exemplary fill and finish workflow, according to aspects of the present disclosure.

FIG. 36 depicts an overview of an exemplary fill and finish workflow, according to aspects of the present disclosure.

FIG. 37 depicts an overview of an exemplary fill and finish workflow, according to aspects of the present disclosure.

FIG. 38 depicts an overview of an overall, combined workflow, according to aspects of the present disclosure.

### CERTAIN DEFINITIONS

About or Approximately: The term “about” or “approximately”, when used herein in reference to a value, refers to a value that is similar, in context to a stated reference value. In general, those skilled in the art, familiar with the context, will appreciate the relevant degree of variance encompassed by “about” or “approximately” in that context. For example, in some embodiments, the term “about” or “approximately” may encompass a range of values that are within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Administration: As used herein, the term “administration” typically refers to the administration of a composition to a subject or system. Those of ordinary skill in the art will be aware of a variety of routes that may, in appropriate circumstances, be utilized for administration to a subject, for example a human. For example, in some embodiments, administration may be ocular, oral, parenteral, topical, etc. In some particular embodiments, administration may be bronchial (e.g., by bronchial instillation), buccal, dermal (which may be or comprise, for example, one or more of topical to the dermis, intradermal, intradermal, transdermal, etc.), enteral, intra-arterial, intradermal, intragastric, intramedullary, intramuscular, intranasal, intraperitoneal, intrathecal, intravenous, intraventricular, within a specific organ (e. g. intrahepatic), mucosal, nasal, oral, rectal, subcutaneous, sublingual, topical, tracheal (e.g., by intratracheal instillation), vaginal, vitreal, etc. In some embodiments, administration may be intramuscular. In some embodiments, administration may involve dosing that is intermittent (e.g., a plurality of doses separated in time) and/or periodic (e.g., individual doses separated by a common period of time) dosing. In some embodiments, administration may involve continuous dosing (e.g., perfusion) for at least a selected period of time.

Agent: In general, the term “agent”, as used herein, is used to refer to an entity (e.g., for example, a lipid, metal, nucleic acid, polypeptide, polysaccharide, small molecule, etc., or complex, combination, mixture or system [e.g., cell, tissue, organism] thereof), or phenomenon (e.g., heat, electric current or field, magnetic force or field, etc.). In appropriate circumstances, as will be clear from context to those skilled in the art, the term may be utilized to refer to an entity that is or comprises a cell or organism, or a fraction, extract, or component thereof. Alternatively or additionally, as context will make clear, the term may be used to refer to a natural product in that it is found in and/or is obtained from nature. In some instances, again as will be clear from context, the term may be used to refer to one or more entities that is man-made in that it is designed, engineered, and/or produced through action of the hand of man and/or is not found in nature. In some embodiments, an agent may be utilized in isolated or pure form; in some embodiments, an agent may be utilized in crude form. In some embodiments, potential agents may be provided as collections or libraries, for example that may be screened to identify or characterize active agents within them. In some cases, the term “agent” may refer to a compound or entity that is or comprises a polymer; in some cases, the term may refer to a compound or entity that comprises one or more polymeric moieties. In some embodiments, the term “agent” may refer to a compound or entity that is not a polymer and/or is substantially free of any polymer and/or of one or more particular polymeric moieties. In some embodiments, the term may refer to a compound or entity that lacks or is substantially free of any polymeric moiety.

Analog: As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance. In some embodiments, an analog is a substance that can be generated through performance of a synthetic process substantially similar to (e.g., sharing a plurality of steps with) one that generates the reference substance. In some embodiments, an analog is or can be generated through performance of a synthetic process different from that used to generate the reference substance.

Antibody agent: As used herein, the term “antibody agent” refers to an agent that specifically binds to a particular antigen. In some embodiments, the term encompasses any polypeptide or polypeptide complex that includes immunoglobulin structural elements sufficient to confer specific binding. Exemplary antibody agents include, but are not limited to monoclonal antibodies or polyclonal antibodies. In some embodiments, an antibody agent may include one or more constant region sequences that are characteristic of mouse, rabbit, primate, or human antibodies. In some embodiments, an antibody agent may include one or more sequence elements are humanized, primatized, chimeric, etc., as is known in the art. In many embodiments, the term “antibody agent” is used to refer to one or more of the art-known or developed constructs or formats for utilizing antibody structural and functional features in alternative presentation. For example, embodiments, an antibody agent utilized in accordance with the present disclosure is in a format selected from, but not limited to, intact IgA, IgG, IgE or IgM antibodies; bi- or multi-specific antibodies (e.g., Zybodies®, etc.); antibody fragments such as Fab fragments, Fab′ fragments, F(ab′)2 fragments, Fd′ fragments, Fd fragments, and isolated complementarity determining regions (CDRs) or sets thereof; single chain Fvs; polypeptide-Fc fusions; single domain antibodies (e.g., shark single domain antibodies such as IgNAR or fragments thereof); cameloid antibodies; masked antibodies (e.g., Probodies®); Small Modular ImmunoPharmaceuticals (“SMIPsTM”); single chain or Tandem diabodies (TandAb®); VHHs; Anticalins®; Nanobodies® minibodies; BiTE®s; ankyrin repeat proteins or DARPINs®; Avimers®; DARTs; TCR-like antibodies; Adnectins®; Affilins®; Trans-bodies®; Affibodies®; TrimerX®; MicroProteins; Fynomers®, Centyrins®; and KALBITOR®s. In some embodiments, an antibody may lack a covalent modification (e.g., attachment of a glycan) that it would have if produced naturally. In some embodiments, an antibody may contain a covalent modification (e.g., attachment of a glycan, a payload [e.g., a detectable moiety, a therapeutic moiety, a catalytic moiety, etc.], or other pendant group [e.g., poly-ethylene glycol, etc.]. In many embodiments, an antibody agent is or comprises a polypeptide whose amino acid sequence includes one or more structural elements recognized by those skilled in the art as a complementarity determining region (CDR); in some embodiments an antibody agent is or comprises a polypeptide whose amino acid sequence includes at least one CDR (e.g., at least one heavy chain CDR and/or at least one light chain CDR) that is substantially identical to one found in a reference antibody. In some embodiments an included CDR is substantially identical to a reference CDR in that it is either identical in sequence or contains between 1-5 amino acid substitutions as compared with the reference CDR. In some embodiments an included CDR is substantially identical to a reference CDR in that it shows at least 85%, 86%, 87%, 88%, 89%, 90%, 91%, 92%, 93%, 94%, 95%, 96%, 97%, 98%, 99%, or 100% sequence identity with the reference CDR. In some embodiments, an included CDR is substantially identical to a reference CDR in that it shows at least 95%, 96%, 97%, 98%, 99%, or 100% sequence identity with the reference CDR. In some embodiments an included CDR is substantially identical to a reference CDR in that at least one amino acid within the included CDR is deleted, added, or substituted as compared with the reference CDR but the included CDR has an amino acid sequence that is otherwise identical with that of the reference CDR. In some embodiments an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an included CDR is substantially identical to a reference CDR in that at least one amino acid within the included CDR is substituted as compared with the reference CDR but the included CDR has an amino acid sequence that is otherwise identical with that of the reference CDR. In some embodiments, an included CDR is substantially identical to a reference CDR in that 1-5 amino acids within the included CDR are deleted, added, or substituted as compared with the reference CDR but the included CDR has an amino acid sequence that is otherwise identical to the reference CDR. In some embodiments, an antibody agent is or comprises a polypeptide whose amino acid sequence includes structural elements recognized by those skilled in the art as an immunoglobulin variable domain. In some embodiments, an antibody agent is a polypeptide protein having a binding domain which is homologous or largely homologous to an immunoglobulin-binding domain.

Antibody agents can be made by the skilled person using methods and commercially available services and kits known in the art. For example, methods of preparation of monoclonal antibodies are well known in the art and include hybridoma technology and phage display technology. Further antibodies suitable for use in the present disclosure are described, for example, in the following publications: Antibodies A Laboratory Manual, Second edition. Edward A. Greenfield. Cold Spring Harbor Laboratory Press (Sep. 30, 2013); Making and Using Antibodies: A Practical Handbook, Second Edition. Eds. Gary C. Howard and Matthew R. Kaser. CRC Press (Jul. 29, 2013); Antibody Engineering: Methods and Protocols, Second Edition (Methods in Molecular Biology). Patrick Chames. Humana Press (Aug. 21, 2012); Monoclonal Antibodies: Methods and Protocols (Methods in Molecular Biology). Eds. Vincent Ossipow and Nicolas Fischer. Humana Press (Feb. 12, 2014); and Human Monoclonal Antibodies: Methods and Protocols (Methods in Molecular Biology). Michael Steinitz. Humana Press (Sep. 30, 2013)).

Antibodies may be produced by standard techniques, for example by immunization with the appropriate polypeptide or portion(s) thereof, or by using a phage display library. If polyclonal antibodies are desired, a selected mammal (e.g., mouse, rabbit, goat, horse, etc.) is immunized with an immunogenic polypeptide bearing a desired epitope(s), optionally haptenized to another polypeptide. Depending on the host species, various adjuvants may be used to increase immunological response. Such adjuvants include, but are not limited to, Freund's, mineral gels such as aluminum hydroxide, and surface-active substances such as lysolecithin, pluronic polyols, polyanions, peptides, oil emulsions, keyhole limpet hemocyanin, and dinitrophenol. Serum from the immunized animal is collected and treated according to known procedures. If serum containing polyclonal antibodies to the desired epitope contains antibodies to other antigens, the polyclonal antibodies can be purified by immunoaffinity chromatography or any other method known in the art. Techniques for producing and processing polyclonal antisera are well known in the art.

Antigen: The term “antigen”, as used herein, refers to an agent that elicits an immune response; and/or (ii) an agent that binds to a T cell receptor (e.g., when presented by an MHC molecule) or to an antibody. In some embodiments, an antigen elicits a humoral response (e.g., including production of antigen-specific antibodies); in some embodiments, an antigen elicits a cellular response (e.g., involving T-cells whose receptors specifically interact with the antigen). In some embodiments, an antigen binds to an antibody and may or may not induce a particular physiological response in an organism. In general, an antigen may be or include any chemical entity such as, for example, a small molecule, a nucleic acid, a polypeptide, a carbohydrate, a lipid, a polymer (in some embodiments other than a biologic polymer [e.g., other than a nucleic acid or amino acid polymer) etc. In some embodiments, an antigen is or comprises a polypeptide. In some embodiments, an antigen is or comprises a glycan. Those of ordinary skill in the art will appreciate that, in general, an antigen may be provided in isolated or pure form, or alternatively may be provided in crude form (e.g., together with other materials, for example in an extract such as a cellular extract or other relatively crude preparation of an antigen-containing source). In some embodiments, antigens utilized in accordance with the present invention are provided in a crude form. In some embodiments, an antigen is a recombinant antigen.

Binding: It will be understood that the term “binding”, as used herein, typically refers to a non-covalent association between or among two or more entities. “Direct” binding involves physical contact between entities or moieties; indirect binding involves physical interaction by way of physical contact with one or more intermediate entities. Binding between two or more entities can typically be assessed in any of a variety of contexts-including where interacting entities or moieties are studied in isolation or in the context of more complex systems (e.g., while covalently or otherwise associated with a carrier entity and/or in a biological system or cell).

Bioreactor: The term “bioreactor” as used herein refers to a vessel used for in vitro transcription described herein. A bioreactor can be of any size so long as it is useful for in vitro transcription. For example, in some embodiments, a bioreactor can be at least 0.5 liter, including 1, 2, 3, 4, 5, 6, 7, 8, 9, 10, 15, 20, 25, 30, 35, 40, 45, 50 liters or more, or any volume in between. The internal conditions of the bioreactor, including, but not limited to pH and temperature, are typically controlled during in vitro transcription. The bioreactor can be composed of any material that is suitable for in vitro transcription under the conditions as described herein, including glass, plastic or metal. One of ordinary skill in the art will be aware of and will be able to choose suitable bioreactor volume for use in practicing in vitro transcription.

Cap: As used herein, the term “cap” refers to a structure comprising or essentially consisting of a nucleoside-5′-triphosphate that is typically joined to a 5′-end of an uncapped RNA (e.g., an uncapped RNA having a 5′-diphosphate). In some embodiments, a cap is or comprises a guanine nucleotide. In some embodiments, a cap is or comprises a naturally-occurring RNA 5′ cap, including, e.g., but not limited to a N7-methylguanosine cap, which has a structure designated as “m7G.” In some embodiments, a cap is or comprises a synthetic cap analog that resembles an RNA cap structure and possesses the ability to stabilize RNA if attached thereto, including, e.g., but not limited to anti-reverse cap analogs (ARCAs) known in the art). Those skilled in the art will appreciate that methods for joining a cap to a 5′ end of an RNA are known in the art. For example, in some embodiments, a capped RNA may be obtained by in vitro capping of RNA that has a 5′ triphosphate group or RNA that has a 5′ diphosphate group with a capping enzyme system (including, e.g., but not limited to vaccinia capping enzyme system or Saccharomyces cerevisiae capping enzyme system). Alternatively, a capped RNA can be obtained by in vitro transcription (IVT) of a DNA template, wherein, in addition to the GTP, an IVT system also contains a cap analog, e.g., as known in the art. Non-limiting examples of a cap analog include a m7GpppG cap analog or an N7-methyl-, 2′-O-methyl-GpppG ARCA cap analog or an N7-methyl-, 3′-O-methyl-GpppG ARCA cap analog, or any commercially available cap analogs, including, e.g., CleanCap (Trilink), EZ Cap, etc., In some embodiments, a cap analog is or comprises a trinucleotide cap analog.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Complementary: As used herein, the term “complementary” is used in reference to oligonucleotide hybridization related by base-pairing rules. For example, the sequence “C-A-G-T” is complementary to the sequence “G-T-C-A.” Complementarity can be partial or total. Thus, any degree of partial complementarity is intended to be included within the scope of the term “complementary” provided that the partial complementarity permits oligonucleotide hybridization. Partial complementarity is where one or more nucleic acid bases is not matched according to the base pairing rules. Total or complete complementarity between nucleic acids is where each and every nucleic acid base is matched with another base under the base pairing rules.

Detecting: The term “detecting” is used broadly herein to include appropriate means of determining the presence or absence of an entity of interest or any form of measurement of an entity of interest in a sample. Thus, “detecting” may include determining, measuring, assessing, or assaying the presence or absence, level, amount, and/or location of an entity of interest. Quantitative and qualitative determinations, measurements or assessments are included, including semi-quantitative. Such determinations, measurements or assessments may be relative, for example when an entity of interest is being detected relative to a control reference, or absolute. As such, the term “quantifying” when used in the context of quantifying an entity of interest can refer to absolute or to relative quantification. Absolute quantification may be accomplished by correlating a detected level of an entity of interest to known control standards (e.g., through generation of a standard curve). Alternatively, relative quantification can be accomplished by comparison of detected levels or amounts between two or more different entities of interest to provide a relative quantification of each of the two or more different entities of interest, i.e., relative to each other.

Determine: Those of ordinary skill in the art, reading the present specification, will appreciate that a step of “determining” can utilize or be accomplished through use of any of a variety of techniques available to those skilled in the art, including for example specific techniques explicitly referred to herein. In some embodiments, determining involves manipulation of a physical sample. In some embodiments, determining involves consideration and/or manipulation of data or information, for example utilizing a computer or other processing unit adapted to perform a relevant analysis. In some embodiments, determining involves receiving relevant information and/or materials from a source. In some embodiments, determining involves comparing one or more features of a sample or entity to a comparable reference.

Dosage form or unit dosage form: Those skilled in the art will appreciate that the term “dosage form” may be used to refer to a physically discrete unit of an active agent (e.g., a therapeutic or diagnostic agent) for administration to a subject. Typically, each such unit contains a predetermined quantity of active agent. In some embodiments, such quantity is a unit dosage amount (or a whole fraction thereof) appropriate for administration in accordance with a dosing regimen that has been determined to correlate with a desired or beneficial outcome when administered to a relevant population (i.e., with a therapeutic dosing regimen). Those of ordinary skill in the art appreciate that the total amount of a therapeutic composition or agent administered to a particular subject is determined by one or more attending physicians and may involve administration of multiple dosage forms.

Encapsulate: The term “encapsulate” or “encapsulation” is used herein to refer to at least a portion of a component is enclosed or surrounded by another material or another component in a composition. In some embodiments, a component can be fully enclosed or surrounded by another material or another component in a composition.

Excipient: As used herein, the term “excipient” refers to a non-therapeutic agent that may be included in a pharmaceutical composition, for example to provide or contribute to a desired property or effect (e.g., desired consistency, delivery, and/or stabilizing effect, etc.). In some embodiments, suitable pharmaceutical excipients to be added to a LNP composition may include, for example, salts, starch, glucose, lactose, sucrose, gelatin, sodium chloride, glycerol, propylene, glycol, water, ethanol and the like.

Encode: As used herein, the term “encode” or “encoding” refers to sequence information of a first molecule that guides production of a second molecule having a defined sequence of nucleotides (e.g., mRNA) or a defined sequence of amino acids. For example, a DNA molecule can encode an RNA molecule (e.g., by a transcription process that includes a DNA-dependent RNA polymerase enzyme). An RNA molecule can encode a polypeptide (e.g., by a translation process). Thus, a gene, a cDNA, or a single-stranded RNA (e.g., an mRNA) encodes a polypeptide if transcription and translation of mRNA corresponding to that gene produces the polypeptide in a cell or other biological system. In some embodiments, a coding region of a single-stranded RNA encoding a target polypeptide agent refers to a coding strand, the nucleotide sequence of which is identical to the mRNA sequence of such a target polypeptide agent. In some embodiments, a coding region of a single-stranded RNA encoding a target polypeptide agent refers to a non-coding strand of such a target polypeptide agent, which may be used as a template for transcription of a gene or cDNA.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Fed-batch process: The term “fed-batch process” as used herein refers to a process in which one or more components are introduced into a vessel, e.g., a bioreactor, at some time subsequent to the beginning of a reaction. In some embodiments, one or more components are introduced by a fed-batch process to maintain its concentration low during a reaction. In some embodiments, one or more components are introduced by a fed-batch process to replenish what is depleted during a reaction.

Five prime untranslated region: As used herein, the terms “five prime untranslated region” or “5′ UTR” refer to a sequence of an mRNA molecule that begins at the transcription start site and ends one nucleotide (nt) before the start codon (usually AUG) of the coding region of an RNA.

Functional: As used herein, a “functional” biological molecule is a biological molecule in a form in which it exhibits a property and/or activity by which it is characterized. In some embodiments, a biological molecule may have two functions (i.e., bifunctional) or many functions (i.e., multifunctional).

Gene: As used herein, the term “gene” refers to a DNA sequence in a chromosome that codes for a product (e.g., an RNA product and/or a polypeptide product). In some embodiments, a gene includes coding sequence (i.e., sequence that encodes a particular product); in some embodiments, a gene includes non-coding sequence. In some particular embodiments, a gene may include both coding (e.g., exonic) and non-coding (e.g., intronic) sequences. In some embodiments, a gene may include one or more regulatory elements that, for example, may control or impact one or more aspects of gene expression (e.g., cell-type-specific expression, inducible expression, etc.).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Homology: As used herein, the term “homology” or “homolog” refers to the overall relatedness between polynucleotide molecules (e.g., DNA molecules and/or RNA molecules) and/or between polypeptide molecules. In some embodiments, polynucleotide molecules (e.g., DNA molecules and/or RNA molecules) and/or polypeptide molecules are considered to be “homologous” to one another if their sequences are at least 15%, 20%, 25%, 30%, 35%, 40%, 45%, 50%, 55%, 60%, 65%, 70%, 75%, 80%, 85%, 90%, 95%, or 99% identical. In some embodiments, polynucleotide molecules (e.g., DNA molecules and/or RNA molecules) and/or polypeptide molecules are considered to be “homologous” to one another if their sequences are at least 25%, 30%, 35%, 40%, 45%, 50%, 55%, 60%, 65%, 70%, 75%, 80%, 85%, 90%, 95%, or 99% similar (e.g., containing residues with related chemical properties at corresponding positions). For example, as is well known by those of ordinary skill in the art, certain amino acids are typically classified as similar to one another as “hydrophobic” or “hydrophilic” amino acids, and/or as having “polar” or “non-polar” side chains. Substitution of one amino acid for another of the same type may often be considered a “homologous” substitution.

Host cell: As used herein, refers to a cell into which exogenous material (e.g., DNA such as recombinant or otherwise) has been introduced. Persons of skill upon reading this disclosure will understand that such terms refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In some embodiments, host cells include prokaryotic and eukaryotic cells selected from any of the Kingdoms of life that are suitable for expressing an exogenous DNA (e.g., a recombinant nucleic acid sequence). Exemplary cells include those of prokaryotes and eukaryotes (single-cell or multiple-cell), bacterial cells (e.g., strains of E. coli, Bacillus spp., Streptomyces spp., etc.), mycobacteria cells, fungal cells, yeast cells (e.g., S. cerevisiae, S. pombe, P. pastoris, P. methanolica, etc.), plant cells, insect cells (e.g., SF-9, SF-21, baculovirus-infected insect cells, Trichoplusia ni, etc.), non-human animal cells, human cells, or cell fusions such as, for example, hybridomas or quadromas. In some embodiments, a host cell is a human, monkey, ape, hamster, rat, or mouse cell. In some embodiments, a host cell is eukaryotic. For example, an eukaryotic host cell may be CHO (e.g., CHO KI, DXB-1 1 CHO, Veggie-CHO), COS (e.g., COS-7), retinal cell, Vero, CV1, kidney (e.g., HEK293, 293 EBNA, MSR 293, MDCK, HaK, BHK), HeLa, HepG2, WI38, MRC 5, Colo205, HB 8065, HL-60, (e.g., BHK21), Jurkat, Daudi, A431 (epidermal), CV-1, U937, 3T3, L cell, C127 cell, SP2/0, NS-0, MMT 060562, Sertoli cell, BRL 3 A cell, HT1080 cell, myeloma cell, tumor cell, or a cell line derived from an aforementioned cell.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4:11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

Improved, increased or reduced: As used herein, these terms, or grammatically comparable comparative terms, indicate values that are relative to a comparable reference measurement. For example, in some embodiments, an assessed value achieved with an agent of interest may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc.). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

Intermediate Storage: The term “intermediate storage” as used herein refers to locally (i.e., at the site or facility) storing of drug substance, drug product (i.e., bulk drug product), solutions, and/or other biological solutions, concentrates, and/or other substances prior to shipment to a CMO (contract manufacturing organization) and/or prior to one or more process steps to be performed as described herein. As used herein, “intermediate storage” is distinguished from “long term storage,” which may refer to off-site storage at, for example, a warehouse or storage facility.

In vitro: The term “in vitro” as used herein refers to events that occur in an artificial environment, e.g., in a test tube or reaction vessel (e.g., a bioreactor), in cell culture, etc., rather than within a multi-cellular organism.

In vitro transcription: As used herein, the term “in vitro transcription” or “IVT” refers to the process whereby transcription occurs in vitro in a non-cellular system to produce a synthetic RNA product for use in various applications, including, e.g., production of protein or polypeptides. Such synthetic RNA products can be translated in vitro or introduced directly into cells, where they can be translated. Such synthetic RNA products include, e.g., but not limited to mRNAs, antisense RNA molecules, shRNA molecules, long non-coding RNA molecules, ribozymes, aptamers, guide RNAs (e.g., for CRISPR), ribosomal RNAs, small nuclear RNAs, small nucleolar RNAs, and the like. An IVT reaction typically utilizes a DNA template (e.g., a linear DNA template) as described and/or utilized herein, ribonucleotides (e.g., non-modified ribonucleotide triphosphates or modified ribonucleotide triphosphates), and an appropriate RNA polymerase.

In vitro transcription RNA composition: As used herein, the term “in vitro transcription RNA composition” refers to a composition comprising target RNA synthesized by in vitro transcription. In some embodiments, such a composition can comprise excess in vitro transcription reagents (including, e.g., ribonucleotides and/or capping agents), nucleic acids or fragments thereof such as DNA templates or fragments thereof, polypeptides or fragments thereof such as recombinant enzymes or host cell proteins or fragments thereof, and/or other impurities. In some embodiments, an in vitro transcription RNA composition may have been treated and/or processed prior to a purification process that ultimately produces an RNA transcript preparation comprising RNA transcript at a desired concentration in an appropriate buffer for formulation and/or further manufacturing and/or processing. For example, in some embodiments, an in vitro transcription RNA composition may have been treated to remove or digest DNA template (e.g., using a DNase). In some embodiments, an in vitro transcription RNA composition may have been treated to remove or digest polypeptides (e.g., enzymes such as RNA polymerases, RNase inhibitors, etc.) present in an in vitro transcription reaction (e.g., using a protease).

In vivo: As used herein, the term “in vivo” refers to events that occur within a multi-cellular organism, such as a human and a non-human animal.

Nanoparticle: As used herein, the term “nanoparticle” refers to a particle having a diameter of less than 1000 nanometers (nm). In some embodiments, a nanoparticle has a diameter of less than 300 nm, as defined by the National Science Foundation. In some embodiments, a nanoparticle has a diameter of less than 100 nm as defined by the National Institutes of Health. In some embodiments, a nanoparticle has a diameter of less than 80 nm as defined by the National Institutes of Health. In some embodiments, a nanoparticle comprises one or more enclosed compartments, separated from the bulk solution by a membrane, which surrounds and encloses a space or compartment.

Nucleic acid/Polynucleotide: As used herein, the term “nucleic acid” refers to a polymer of at least 2 nucleotides or more, including, e.g., at least 3 nucleotides, at least 4 nucleotides, at least 5 nucleotides, at least 6 nucleotides, at least 7 nucleotides, at least 8 nucleotides, at least 9 nucleotides, at least 10 nucleotides, or more. In some embodiments, a nucleic acid is or comprises DNA. In some embodiments, a nucleic acid is or comprises RNA. In some embodiments, a nucleic acid is or comprises peptide nucleic acid (PNA). In some embodiments, a nucleic acid is or comprises a single stranded nucleic acid. In some embodiments, a nucleic acid is or comprises a double-stranded nucleic acid. In some embodiments, a nucleic acid comprises both single and double-stranded portions. In some embodiments, a nucleic acid comprises a backbone that comprises one or more phosphodiester linkages. In some embodiments, a nucleic acid comprises a backbone that comprises both phosphodiester and non-phosphodiester linkages. For example, in some embodiments, a nucleic acid may comprise a backbone that comprises one or more phosphorothioate or 5′-N-phosphoramidite linkages and/or one or more peptide bonds, e.g., as in a “peptide nucleic acid”. In some embodiments, a nucleic acid comprises one or more, or all, natural residues (e.g., adenine, cytosine, deoxyadenosine, deoxycytidine, deoxyguanosine, deoxythymidine, guanine, thymine, uracil). In some embodiments, a nucleic acid comprises on or more, or all, non-natural residues. In some embodiments, a non-natural residue comprises a nucleoside analog (e.g., 2-aminoadenosine, 2-thiothymidine, inosine, pyrrolo-pyrimidine, 3-methyl adenosine, 5-methylcytidine, C-5 propynyl-cytidine, 1-methyl-pseudouridine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 6-O-methylguanine, 2-thiocytidine, methylated bases, intercalated bases, and combinations thereof). In some embodiments, a non-natural residue comprises one or more modified sugars (e.g., 2′-fluororibose, ribose, 2′-deoxyribose, arabinose, and hexose) as compared to those in natural residues. In some embodiments, a nucleic acid has a nucleotide sequence that encodes a functional gene product such as an RNA or polypeptide. In some embodiments, a nucleic acid has a nucleotide sequence that comprises one or more introns. In some embodiments, a nucleic acid may be prepared by isolation from a natural source, enzymatic synthesis (e.g., by polymerization based on a complementary template, e.g., in vivo or in vitro, reproduction in a recombinant cell or system, or chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 1 10, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000, 5500, 6000, 6500, 7000, 7500, 8000, 8500, 9000, 9500, 10,000, 10,500, 11,000, 11,500, 12,000, 12,500, 13,000, 13,500, 14,000, 14,500, 15,000, 15,500, 16,000, 16,500, 17,000, 17,500, 18,000, 18,500, 19,000, 19,500, or 20,000 or more residues or nucleotides long.

Pharmaceutical grade: The term “pharmaceutical grade” as used herein refers to standards for chemical and biological drug substances, drug products, dosage forms, compounded preparations, excipients, medical devices, and dietary supplements, established by a recognized national or regional pharmacopeia (e.g., The United States Pharmacopeia and The Formulary (USP-NF)).

Polypeptide: The term “polypeptide”, as used herein, typically has its art-recognized meaning of a polymer of at least three amino acids or more. Those of ordinary skill in the art will appreciate that the term “polypeptide” is intended to be sufficiently general as to encompass not only polypeptides having a complete sequence recited herein, but also to encompass polypeptides that represent functional, biologically active, or characteristic fragments, portions or domains (e.g., fragments, portions, or domains retaining at least one activity) of such complete polypeptides. In some embodiments, polypeptides may contain L-amino acids, D-amino acids, or both and/or may contain any of a variety of amino acid modifications or analogs known in the art. Useful modifications include, e.g., terminal acetylation, amidation, methylation, etc. In some embodiments, polypeptides may comprise natural amino acids, non-natural amino acids, synthetic amino acids, and combinations thereof (e.g., may be or comprise peptidomimetics). In some embodiments, a polypeptide may be or comprise an enzyme. In some embodiments, a polypeptide may be or comprise a polypeptide antigen. In some embodiments, a polypeptide may be or comprise an antibody agent. In some embodiments a polypeptide may be or comprise a cytokine.

Pure or Purified: As used herein, an agent or entity is “pure” or “purified” if it is substantially free of other components. For example, a preparation that contains more than about 90% of a particular agent or entity is typically considered to be a pure preparation. In some embodiments, an agent or entity is at least 91%, at least 92%, at least 93%, at least 94%, at least 95%, at least 96%, at least 97%, at least 98%, or at least 99% pure in a preparation.

Ribonucleotide: As used herein, the term “ribonucleotide” encompasses unmodified ribonucleotides and modified ribonucleotides. For example, unmodified ribonucleotides include the purine bases adenine (A) and guanine (G), and the pyrimidine bases cytosine (C) and uracil (U). Modified ribonucleotides may include one or more modifications including, but not limited to, for example, (a) end modifications, e.g., 5′ end modifications (e.g., phosphorylation, dephosphorylation, conjugation, inverted linkages, etc.), 3′ end modifications (e.g., conjugation, inverted linkages, etc.), (b) base modifications, e.g., replacement with modified bases, stabilizing bases, destabilizing bases, or bases that base pair with an expanded repertoire of partners, or conjugated bases, (c) sugar modifications (e.g., at the 2′ position or 4′ position) or replacement of the sugar, and (d) internucleoside linkage modifications, including modification or replacement of the phosphodiester linkages. The term “ribonucleotide” also encompasses ribonucleotide triphosphates including modified and non-modified ribonucleotide triphosphates.

Ribonucleic acid (RNA): As used herein, the term “RNA” refers to a polymer of ribonucleotides. In some embodiments, an RNA is single stranded. In some embodiments, an RNA is double stranded. In some embodiments, an RNA comprises both single and double stranded portions. In some embodiments, an RNA can comprise a backbone structure as described in the definition of “Nucleic acid/Polynucleotide” above. An RNA can be a regulatory RNA (e.g., siRNA, microRNA, etc.), or a messenger RNA (mRNA). In some embodiments, an RNA is a mRNA. In some embodiments, where an RNA is a mRNA, a RNA typically comprises at its 3′ end a poly(A) region. In some embodiments where an RNA is a mRNA, an RNA typically comprises at its 5′ end, an art-recognized cap structure, e.g., for recognizing and attachment of a mRNA to a ribosome to initiate translation. In some embodiments, an RNA is a synthetic RNA. Synthetic RNAs include RNAs that are synthesized in vitro (e.g., by enzymatic synthesis methods and/or by chemical synthesis methods). In some embodiments, an RNA is a single-stranded RNA. In some embodiments, a single-stranded RNA may comprise self-complementary elements and/or may establish a secondary and/or tertiary structure. One of ordinary skill in the art will understand that when a single-stranded RNA is referred to as “encoding,” it can mean that it comprises a nucleic acid sequence that itself encodes or that it comprises a complement of the nucleic acid sequence that encodes. In some embodiments, a single-stranded RNA can be a self-amplifying RNA (also known as self-replicating RNA).

Recombinant: as used herein, is intended to refer to polypeptides that are designed, engineered, prepared, expressed, created, manufactured, and/or or isolated by recombinant means, such as polypeptides expressed using a recombinant expression vector transfected into a host cell; polypeptides isolated from a recombinant, combinatorial human polypeptide library; polypeptides isolated from an animal (e.g., a mouse, rabbit, sheep, fish, etc.) that is transgenic for or otherwise has been manipulated to express a gene or genes, or gene components that encode and/or direct expression of the polypeptide or one or more component(s), portion(s), element(s), or domain(s) thereof; and/or polypeptides prepared, expressed, created or isolated by any other means that involves splicing or ligating selected nucleic acid sequence elements to one another, chemically synthesizing selected sequence elements, and/or otherwise generating a nucleic acid that encodes and/or directs expression of the polypeptide or one or more component(s), portion(s), element(s), or domain(s) thereof. In some embodiments, one or more of such selected sequence elements is found in nature. In some embodiments, one or more of such selected sequence elements is designed in silico. In some embodiments, one or more such selected sequence elements results from mutagenesis (e.g., in vivo or in vitro) of a known sequence element, e.g., from a natural or synthetic source such as, for example, in the germline of a source organism of interest (e.g., of a human, a mouse, etc.).

Reference: As used herein, the term “reference” describes a standard or control relative to which a comparison is performed. For example, in some embodiments, an agent, animal, individual, population, sample, sequence or value of interest is compared with a reference or control agent, animal, individual, population, sample, sequence or value. In some embodiments, a reference or control is tested and/or determined substantially simultaneously with the testing or determination of interest. In some embodiments, a reference or control is a historical reference or control, optionally embodied in a tangible medium. Typically, as would be understood by those skilled in the art, a reference or control is determined or characterized under comparable conditions or circumstances to those under assessment. Those skilled in the art will appreciate when sufficient similarities are present to justify reliance on and/or comparison to a particular possible reference or control.

RNA polymerase: As used herein, the term “RNA polymerase” refers to an enzyme that catalyzes polyribonucleotide synthesis by addition of ribonucleotide units to a nucleotide chain using DNA or RNA as a template. The term refers to either a complete enzyme as it occurs in nature, or an isolated, active catalytic or functional domain, or fragment thereof. In some embodiments, an RNA polymerase enzyme initiates synthesis at the 3′-end of a primer or a nucleic acid strand, or at a promoter sequence, and proceeds in the 5′-direction along the target nucleic acid to synthesize a strand complementary to the target nucleic acid until synthesis terminates.

RNA transcript preparation: The term “RNA transcript preparation” as used herein refers to a preparation comprising RNA transcript that is purified from an in vitro transcription RNA composition described herein. In some embodiments, an RNA transcript preparation is a preparation comprising pharmaceutical-grade RNA transcript. In some embodiments, an RNA transcript preparation is a preparation comprising RNA transcript, where one or more product quality attributes are characterized and determined to meet a release and/or acceptance criteria (e.g., as described herein). Examples of such product quality attributes include, but are not limited to appearance, RNA length, identity of drug substance as RNA, RNA integrity, RNA sequence, RNA concentration, pH, osmolality, residual DNA template, residual double stranded RNA, bacterial endotoxins, bioburden, and combinations thereof.

Room temperature: As used herein, the term “room temperature” refers to an ambient temperature. In some embodiments, a room temperature is about 18° C.-30° C., e.g., about 18° C.-25° C., or about 20° C.-25° C., or about 20-30° C., or about 23-27° C. or about 25° C.

Sample: As used herein, the term “sample” typically refers to an aliquot of material obtained or derived from a source of interest, e.g., as described herein. In some embodiments, a source of interest is a biological or environmental source. In some embodiments, a source of interest may be or comprise a cell or an organism, such as a microbe, a plant, or an animal (e.g., a mouse). In some embodiments, a source of interest is or comprises biological tissue or fluid. In some embodiments, a biological fluid may be or comprise an intracellular fluid, an extracellular fluid, an intravascular fluid (blood plasma), an interstitial fluid, a lymphatic fluid, and/or a transcellular fluid. In some embodiments, a biological tissue or sample may be obtained, for example, by aspirate, biopsy (e.g., fine needle or tissue biopsy), swab (e.g., oral, nasal, skin, or vaginal swab), scraping, surgery, washing or lavage (e.g., bronchoalveolar, ductal, nasal, ocular, oral, uterine, vaginal, or other washing or lavage). In some embodiments, a sample is or comprises cells obtained from a subject. In some embodiments, a sample is a “primary sample” obtained directly from a source of interest by any appropriate means. In some embodiments, as will be clear from context, the term “sample” refers to a preparation that is obtained by processing (e.g., by removing one or more components of and/or by adding one or more agents to) a primary sample. For example, a “processed sample” may comprise, for example nucleic acids or proteins extracted from a sample or obtained by subjecting a primary sample to one or more techniques such as amplification or reverse transcription of nucleic acid, isolation and/or purification of certain components, etc.

Stable: The term “stable,” when applied to nucleic acids and/or compositions comprising nucleic acids, e.g., encapsulated in lipid nanoparticles, means that such nucleic acids and/or compositions maintain one or more aspects of their characteristics (e.g., physical and/or structural characteristics, function, and/or activity) over a period of time under a designated set of conditions (e.g., pH, temperature, light, relative humidity, etc.). In some embodiments, such stability is maintained over a period of time of at least about one hour; in some embodiments, such stability is maintained over a period of time of about 5 hours, about 10 hours, about one (1) day, about one (1) week, about two (2) weeks, about one (1) month, about two (2) months, about three (3) months, about four (4) months, about five (5) months, about six (6) months, about eight (8) months, about ten (10) months, about twelve (12) months, about twenty-four (24) months, about thirty-six (36) months, or longer. In some embodiments, such stability is maintained over a period of time within the range of about one (1) day to about twenty-four (24) months, about two (2) weeks to about twelve (12) months, about two (2) months to about five (5) months, etc. In some embodiments, such stability is maintained under an ambient condition (e.g., at room temperature and ambient pressure). In some embodiments, such stability is maintained under a physiological condition (e.g., in vivo or at about 37° C. for example in serum or in phosphate buffered saline). In some embodiments, such stability is maintained under cold storage (e.g., at or below about 4° C., including, e.g., −20° C., or −70° C.). In some embodiments, such stability is maintained when nucleic acids and/or compositions comprising the same are protected from light (e.g., maintaining in the dark).

As an example, in some embodiments, the term “stable” is used in reference to a nanoparticle composition (e.g., a lipid nanoparticle composition). In such embodiments, a stable nanoparticle composition (e.g., a stable nanoparticle composition) and/or component(s) thereof maintain one or more aspects of its characteristics (e.g., physical and/or structural characteristics, function(s), and/or activity) over a period of time under a designated set of conditions. For example, in some embodiments, a stable nanoparticle composition (e.g., a lipid nanoparticle composition) is characterized in that average particle size, particle size distribution, and/or polydispersity of nanoparticles is substantially maintained (e.g., within 10% or less, as compared to the initial characteristic(s)) over a period of time (e.g., as described herein) under a designated set of conditions (e.g., as described herein). In some embodiments, a stable nanoparticle composition (e.g., a lipid nanoparticle composition) is characterized in that no detectable amount of degradation products (e.g., associated with hydrolysis and/or enzymatic digestion) is present after it is maintained under a designated set of conditions (e.g., as described herein) over a period of time.

Synthetic: As used herein, the term “synthetic” refers to an entity that is artificial, or that is made with human intervention, or that results from synthesis rather than naturally occurring. For example, in some embodiments, a synthetic nucleic acid or polynucleotide refers to a nucleic acid molecule that is chemically synthesized, e.g., in some embodiments by solid-phase synthesis. In some embodiments, the term “synthetic” refers to an entity that is made outside of biological cells. For example, in some embodiments, a synthetic nucleic acid or polynucleotide refers to a nucleic acid molecule (e.g., an RNA) that is produced by in vitro transcription using a template.

Three prime untranslated region: As used herein, the terms “three prime untranslated region” or “3′ UTR” refer to the sequence of an mRNA molecule that begins following the stop codon of the coding region of an open reading frame sequence. In some embodiments, the 3′ UTR begins immediately after the stop codon of the coding region of an open reading frame sequence. In other embodiments, the 3′ UTR does not begin immediately after stop codon of the coding region of an open reading frame sequence.

Threshold level (e.g., acceptance criteria): As used herein, the term “threshold level” refers to a level that are used as a reference to attain information on and/or classify the results of a measurement, for example, the results of a measurement attained in an assay. For example, in some embodiments, a threshold level means a value measured in an assay that defines the dividing line between two subsets of a population (e.g., a batch that satisfy quality control criteria vs. a batch that does not satisfy quality control criteria). Thus, a value that is equal to or higher than the threshold level defines one subset of the population, and a value that is lower than the threshold level defines the other subset of the population. A threshold level can be determined based on one or more control samples or across a population of control samples. A threshold level can be determined prior to, concurrently with, or after the measurement of interest is taken. In some embodiments, a threshold level can be a range of values.

Vector: As used herein, the term “vector” refers to a nucleic acid molecule capable of transporting another nucleic acid to which it has been linked. One type of vector is a “plasmid”, which refers to a circular double stranded DNA into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “expression vectors.”

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Green and Sambrook, Molecular Cloning: A Laboratory Manual (4th ed., Cold Spring Harbor Laboratory Press, Cold Spring Harbor, N.Y. (2012)), which is incorporated herein by reference for any purpose.

### DETAILED DESCRIPTION OF CERTAIN EMBODIMENTS

Nucleic acid therapeutics, and particularly RNA therapeutics represent a particularly promising class of therapies for treatment and prevention of various diseases such as cancer, infectious diseases, and/or diseases or disorders associated with overabundance or deficiency in certain proteins.

RNA therapeutics provide remarkably effective as vaccines to address the COVID19 pandemic. Particularly given the promise of this technology, and its adaptability to a wide variety of clinical contexts, including massively large scale (e.g., vaccination and/or treatment on a global scale such as is under development for SARS-CoV-2), improvements to manufacturing technologies, especially those applicable to large-scale production, are especially valuable.

Development of effective delivery technologies has been central to the success of nucleic acid therapeutics, and lipid nanoparticle technologies have proven to be particularly effective (reviewed in, for example, Cullis et al. Molecular Therapy 25:1467 Jul. 5, 2017; See also, U.S. Pat. No. 8,058,069), specifically including for RNA therapeutics (reviewed in, for example, Hou et al., Nat. Rev. Mater doi.org/10.1038/s41578-021-00358-0, Aug. 10, 2021).

Technologies provided herein are useful, among other things, to achieve particularly effective and/or efficient production, e.g., on commercial scale and/or under commercial conditions, of pharmaceutical grade LNP preparations and/or compositions (e.g., nucleic acid-LNP preparations, and specifically RNA-LNP preparations). For example, in various embodiments, provided technologies permit and/or facilitate achievement of requirements unique to pharmaceutical-grade (and/or scale) production such as, for example, batch size and/or rate of production, pre-determined in-process controls and/or lot release specifications (e.g., high purity, integrity, potency, and/or stability, etc.), etc.

The present disclosure provides technologies for manufacturing LNP compositions (e.g., including RNA, e.g., therapeutic RNA such as therapeutic mRNA). In some embodiments, provided technologies are useful for manufacturing pharmaceutical-grade RNA-LNP therapeutics.

In some embodiments, provided technologies are useful for large scale manufacturing of LNP (e.g., nucleic acid-LNP, e.g., RNA-LNP) therapeutics, e.g., pharmaceutical-grade therapeutics. For example, in some such embodiments, technologies provided herein can be used to produce a pharmaceutical-grade batch throughput of at least 10,000 vials of LNP (e.g., nucleic acid-LNP, e.g., RNA-LNP) therapeutics (including, e.g., at least 20,000 vials, at least 30,000 vials, at least 40,000 vials, at least 50,000 vials, at least 60,000 vials, at least 70,000 vials, at least 80,000 vials, at least 90,000 vials, at least 100,000 vials, at least 200,000 vials, at least 300,000 vials, at least 400,000 vials, at least 500,000 vials, or more). For example, in some such embodiments, technologies provided herein can be used to produce a pharmaceutical-grade batch throughput of at least 50 L of LNP (e.g., nucleic acid-LNP, e.g., RNA-LNP) therapeutics (including e.g., at least 50 L, at least 60 L, at least 70 L, at least 80 L, at least 100 L, at least 110 L, at least 120 L, at least 130 L, at least 140 L, at least 150 L or more. In some embodiments, each vial can comprise an RNA drug product in an amount of 0.01 mg to 0.5 mg (e.g., 0.01 mg, 0.02 mg, 0.03 mg, 0.04 mg, 0.05 mg, 0.06 mg, 0.07 mg, 0.08 mg, 0.09 mg, 0.1 mg, 0.15 mg, 0.2 mg, 0.25 mg, 0.3 mg, 0.35 mg, 0.4 mg, 0.45 mg, 0.5 mg).

Technologies described herein can be useful for manufacturing LNP (e.g., nucleic acid-LNP, e.g., RNA-LNP) compositions for treatment and/or prevention of a disease, disorder, or condition (e.g., cancer, infectious diseases, diseases associates with protein deficiency, etc.). In some embodiments, technologies described herein can be useful for manufacturing LNP (e.g., nucleic acid-LNP, e.g., RNA-LNP) compositions that comprise or deliver (e.g., by comprising and/or delivering a nucleic acid, such as an RNA, that encodes it) a polypeptide.

In some embodiments, technologies described herein can be useful for manufacturing LNP (e.g., nucleic acid-LNP, e.g., RNA-LNP) compositions for inducing an immune response to an antigen. In some embodiments, technologies described herein can be useful for manufacturing LNP (e.g., nucleic acid-LNP, e.g., RNA-LNP) compositions for treatment and/or prevention of coronavirus infection, e.g., SARS-CoV-2 infection, as described in Walsh et al. “RNA-based COVID-19 vaccine BNT162b2 selected for a pivotal efficacy study” medRxiv preprint (2020), which is online accessible at: https://doi.org/10.1101/2020.08.17.20176651; and Milligan et al. “Phase I/II study of COVID-19 RNA vaccine BNT162b1 in adults” Nature (2020 August), which is online accessible at: https://doi.org/10.1038/s41586-020-2639-4, the contents of each of which are incorporated by reference in their entirety.

### Lipid Nanoparticles

Those skilled in the art are aware that lipid nanoparticles have achieved successful clinical delivery of a wide range of therapeutic agents including, for example, small molecules, and various nucleic acids—e.g., oligonucleotides, siRNAs, and mRNAs (reviewed, for example, in Hu et al., Nat. Rev. Mater. https://doi.org/10.1038/s41578-021-00358-0, Aug. 10, 2021).

Various routes of administration for lipid nanoparticle compositions have been proposed and/or tested; those skilled in the art will be aware of appropriate routes for particular compositions (e.g., depending on agent being delivered). To give but a few examples, in some embodiments, LNPs are parenterally administered; most clinical studies have utilized parenteral administration, and particularly intravenous, subcutaneous, intradermal, intravitreal, intratumoral, or intramuscular injection. Intrautero injection has also been described. In some embodiments, topical administration is utilized. In some embodiments, intranasal administration is utilized.

In some embodiments, administered LNPs are delivered to or accumulate in the liver. Given that the liver is naturally effective at producing and secreting proteins, liver delivery can prove useful for achieving delivery of an LNP-encapsulated agent (and/or, in the case of a nucleic acid agent such as an RNA agent, a polypeptide encoded thereby) into the bloodstream. Such liver delivery has been proposed to be particularly useful, for example, for expression of proteins that are missing in certain metabolic or hematological disorders, or that are effective in provoking immune responses (e.g., particularly antibody responses), for example against infectious agents or cancer cells.

In some embodiments, administered LNPs are delivered to and/or taken up by antigen-presenting cells (e.g., as may be present in skin, muscle, mucosal tissues, etc.); such administration may be particularly useful or effective for induction of T cell immunity (e.g., for treatment of infectious diseases and/or cancers).

In various embodiments, lipid nanoparticles can have an average size (e.g., mean diameter) of about 30 nm to about 150 nm, about 40 nm to about 150 nm, about 50 nm to about 150 nm, about 50 nm to about 130 nm, about 50 nm to about 110 nm, about 50 nm to about 100 nm, about 50 to about 90 nm, or about 60 nm to about 80 nm, or about 60 nm to about 70 nm. In some embodiments, lipid nanoparticles that may be useful in accordance with the present disclosure can have an average size (e.g., mean diameter) of about 50 nm to about 100 nm. In some embodiments, lipid nanoparticles may have an average size (e.g., mean diameter) of less than 80 nm, less than 75 nm, less than 70 nm, less than 65 nm, less than 60 nm, less than 55 nm, less than 50 nm, or less than 45 nm. In some embodiments, lipid nanoparticles that may be useful in accordance with the present disclosure can have an average size (e.g., mean diameter) of about 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm.

In some embodiments, lipids that form lipid nanoparticles described herein comprise: a polymer-conjugated lipid; a cationic lipid; and a helper neutral lipid. In some such embodiments, total polymer-conjugated lipid may be present in about 0.5-5 mol %, about 0.7-3.5 mol %, about 1-2.5 mol %, about 1.5-2 mol %, or about 1.5-1.8 mol % of the total lipids. In some embodiments, total polymer-conjugated lipid may be present in about 1-2.5 mol % of the total lipids. In some embodiments, the molar ratio of total cationic lipid to total polymer-conjugated lipid (e.g., PEG-conjugated lipid) may be about 100:1 to about 20:1, or about 50:1 to about 20:1, or about 40:1 to about 20:1, or about 35:1 to about 25:1. In some embodiments, the molar ratio of total cationic lipid to total polymer-conjugated lipid may be about 35:1 to about 25:1.

In some embodiments involving a polymer-conjugated lipid, a cationic lipid, and a helper neutral lipid in lipid nanoparticles described herein, total cationic lipid is present in about 35-65 mol %, about 40-60 mol %, about 41-49 mol %, about 41-48 mol %, about 42-48 mol %, about 43-48 mol %, about 44-48 mol %, about 45-48 mol %, or about 46-49 mol % of the total lipids. In certain embodiments, total cationic lipid is present in about 47.0, 47.1, 47.2, 47.3, 47.4, 47.5, 47.6, 47.7, 47.8, 47.9 or 48.0 mol % of the total lipids.

In some embodiments involving a polymer-conjugated lipid, a cationic lipid, and a helper neutral lipid in lipid nanoparticles described herein, total neutral lipid is present in about 35-65 mol %, about 40-60 mol %, about 45-55 mol %, or about 47-52 mol % of the total lipids. In some embodiments, total neutral lipid is present in 35-65 mol % of the total lipids. In some embodiments, total non-steroid neutral lipid (e.g., DPSC) is present in about 5-15 mol %, about 7-13 mol %, or 9-11 mol % of the total lipids. In some embodiments, total non-steroid neutral lipid is present in about 9.5, 10 or 10.5 mol % of the total lipids. In some embodiments, the molar ratio of the total cationic lipid to the non-steroid neutral lipid ranges from about 4.1:1.0 to about 4.9:1.0, from about 4.5:1.0 to about 4.8:1.0, or from about 4.7:1.0 to 4.8:1.0. In some embodiments, total steroid neutral lipid (e.g., cholesterol) is present in about 35-50 mol %, about 39-49 mol %, about 39-46 mol %, about 39-44 mol %, or about 39-42 mol % of the total lipids. In certain embodiments, total steroid neutral lipid (e.g., cholesterol) is present in about 39, 40, 41, 42, 43, 44, 45, or 46 mol % of the total lipids. In certain embodiments, the molar ratio of total cationic lipid to total steroid neutral lipid is about 1.5:1 to 1:1.2, or about 1.2:1 to 1:1.2.

In some embodiments, a lipid composition comprising a cationic lipid, a polymer-conjugated lipid, and a neutral lipid can have individual lipids present in certain molar percents of the total lipids, or in certain molar ratios (relative to each other) as described in WO 2018/081480, the entire contents of each of which are incorporated herein by reference for the purposes described herein.

In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2.5 mol % of the total lipids; the cationic lipid is present in 35-65 mol % of the total lipids; and the neutral lipid is present in 35-65 mol % of the total lipids. In some embodiments, lipids that form the lipid nanoparticles comprise: a polymer-conjugated lipid (e.g., PEG-conjugated lipid); a cationic lipid; and a neutral lipid, wherein the polymer-conjugated lipid is present in about 1-2 mol % of the total lipids; the cationic lipid is present in 45-48.5 mol % of the total lipids; and the neutral lipid is present in 45-55 mol % of the total lipids. In some embodiments, lipids that form the lipid nanoparticles comprise: a polymer-conjugated lipid (e.g., PEG-conjugated lipid); a cationic lipid; and a neutral lipid comprising a non-steroid neutral lipid and a steroid neutral lipid, wherein the polymer-conjugated lipid is present in about 1-2 mol % of the total lipids; the cationic lipid is present in 45-48.5 mol % of the total lipids; the non-steroid neutral lipid is present in 9-11 mol % of the total lipids; and the steroid neutral lipid is present in about 36-44 mol % of the total lipids. In many of such embodiments, a PEG-conjugated lipid is or comprises a structure

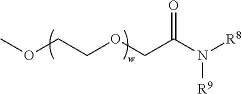

as described in WO 2017/075531 (also described above), or a derivative thereof. In some embodiments, a PEG-conjugated lipid is or comprises 2-[(polyethylene glycol)-2000]-N,N-ditetradecylacetamide. In many of such embodiments, a cationic lipid is or comprises a chemical structure selected from I-1 to I-10 of Table 1 herein or a derivative thereof. In some embodiments, a cationic lipid is or comprises ((4-hydroxybutyl) azanediyl)bis(hexane-6,1-diyl)bis(2-hexyldecanoate). In many of such embodiments, a neutral lipid comprises DSPC and cholesterol, wherein DSPC is a non-steroid neutral lipid and cholesterol is a steroid neutral lipid.

In some embodiments, lipid nanoparticles include one or more cationic lipids (e.g., ones described herein). In some embodiments, cationic lipid nanoparticles may comprise at least one cationic lipid, at least one polymer-conjugated lipid, and at least one helper lipid (e.g., at least one neutral lipid).

### Liposomes

Those skilled in the art are aware that liposomes have also achieved successful clinical delivery of a wide range of therapeutic agents. In some embodiments, liposomes may include both a hydrophilic region and a hydrophobic region. In some embodiments, liposomes may include a spherical lipid bilayer. In some embodiments, liposomes may include phospholipids (e.g., phosphatidylcholine and/or cholesterol). In some embodiments, liposomes may include phosphatidylethanolamine. Liposomes can be used in connection with drug delivery.

### Lipoplexes

Those skilled in the art are aware that lipoplexes have also achieved successful clinical delivery of a wide range of therapeutic agents. In some embodiments, lipoplexes include cationic lipids. In certain aspects according to the present embodiments, cationic lipids are structured to allow or enable electrostatic interaction negatively charges (e.g., negatively charged phosphate backbones (e.g., of nucleic acids)). Accordingly, lipoplexes may be used in connection with the delivery of nucleic acids (i.e., for therapeutic benefit and/or in connection with drug/vaccine delivery).

In the context of the present disclosure, the terms “lipoplex” and “RNA lipoplex particle” relates to a particle that contains lipid, in particular cationic lipid, and RNA. Electrostatic interactions between positively charged liposomes and negatively charged RNA results in complexation and spontaneous formation of RNA lipoplex particles. Positively charged liposomes may be generally synthesized using a cationic lipid, such as DOTMA (dioleoyl-3-trimethylammonium propane), and additional lipids, such as DOPE (dioleoylphosphatidylethanolamine). In some embodiments, a lipid preparation or solution includes 0.6 mM DOTMA, DOPE, ethanol, and acetic acid. In one embodiment, an RNA lipoplex particle is a nanoparticle. The disclosures of U.S. Pat. Nos. 11,173,120 and 11,395,799 are incorporated herein by reference, in their entireties.

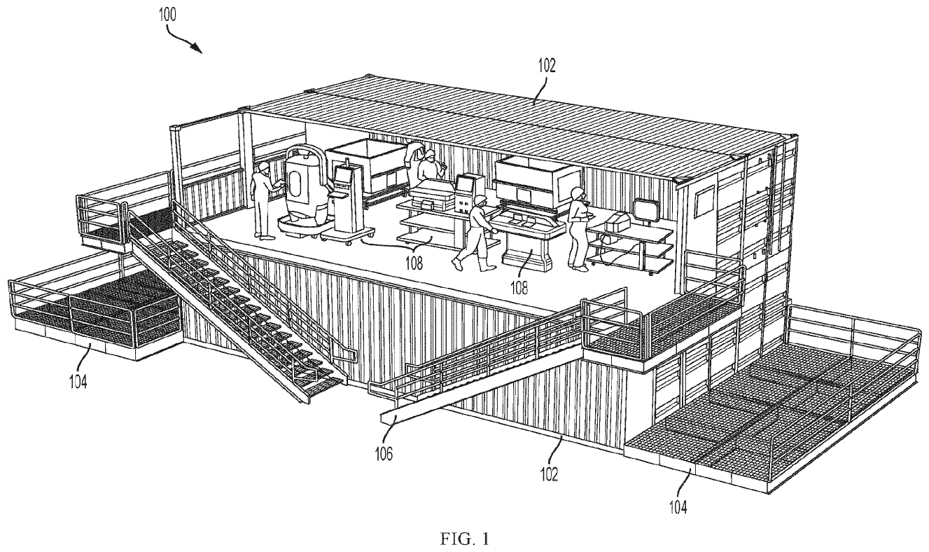

FIG. 1 depicts an exemplary modular drug production system 100, according to aspects of the present disclosure. In the embodiment of FIG. 1, the system 100 may include a total of five or six shipping containers 102 with three shipping containers 102 on the bottom and another two or three shipping containers 102 stacked on top of the bottom three (3) containers. The system 100 may include various external platforms 104 and/or staircases 106 as necessary to allow accessibility of personnel within the system 100. The system 100 includes various equipment 108 required for drug production. The equipment 108 may include tanks, filtration equipment, mixers, valves, sensors, process control and testing equipment (including computers), as well as other equipment. In some embodiments, the system 100 may include HVAC (heating, ventilation, and air conditioning) equipment to maintain the system 100 within a desired temperature range (for example, from about 15° C. to about 25° C., and/or from about 10° C. to about 30° C., as well as other suitable temperature ranges). In one or more embodiments, the system may include a first set of three containers 102 stacked on top of a second set of three containers 102 with the first set of three containers 102 containing a drug substance production module and/or a drug product production module, and with the second set of three containers containing HVAC equipment. In some embodiments, the HVAC equipment may be contained on the top level with the drug substance production module and/or a drug product production module contained on the bottom level. In some embodiments, the relative positions may be reversed (i.e., with the HVAC equipment on the bottom).

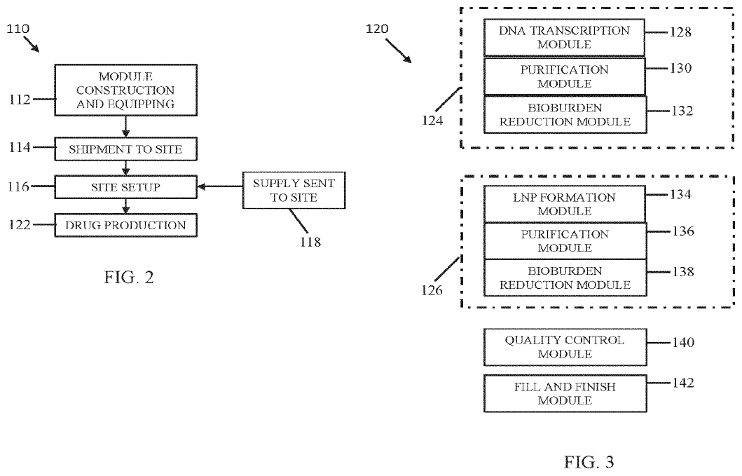

FIG. 2 depicts an overview of an exemplary drug product manufacturing enterprise 110 and/or process, according to aspects of the present disclosure. In the embodiment of FIG. 2, the enterprise 110 and/or process 110 may include constructing modules and equipping the modules (i.e., containers 102) at step 112 at a production facility (for example, at a BioNTech facility in Germany, and/or at one or more contractor and/or partner facilities located at various locations). At step 114, the enterprise 110 and/or process 110 may include shipping the constructed containers 102 and/or equipment to a drug production site. At step 116, the enterprise 110 and/or process 110 may include setting up the constructed modules (i.e.m containers 102) and/or equipment at the drug production site. At step 118, the enterprise 110 and/or process 110 may include shipping supplies (i.e., process input materials that are needed for producing drug substances and/or drug products) to the drug production site. In some embodiments, step 118 may occur concurrent with step 114 (that is, process input supplies are shipped to the site with the containers 102). At step 122, the enterprise 110 and/or process 110 may include initiating the production of drug substances and/or drug products.

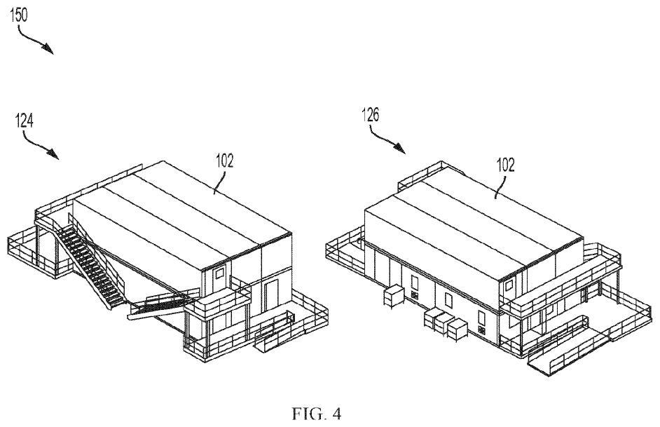

FIG. 3 depicts an overview of an exemplary drug product manufacturing site, system and/or process 120, according to aspects of the present disclosure. In the embodiment of FIG. 3, the system 120 may generally include 8 containers 102. In some embodiments, the system 120 may include less than 8 containers 102 and/or more than 8 containers 102. The system 120 may include a first group of one or more containers 102 dedicated to drug substance production 124 as well as a second group of one or more containers 102 dedicated to drug product production 126. In addition, the system 120 may include a quality control module (and/or container) 140 as well as a fill and finish module (and/or container) 142. The first group of one or more containers 102 dedicated to drug substance production 124 may include a DNA transcription module 128 (or container), a first purification module 130 (or container), and a first bioburden reduction module 132 (or container). The second group of one or more containers 102 dedicated to drug product production 126 may include an LNP formation module 134 (or container), a second purification module 136 (or container), and a second bioburden reduction module 138 (or container). Each module or container may receive inputs from the previous or adjacent module. For example, the LNP formation module 134 may receive drug substance as an input from the first bioburden reduction module 132.

FIG. 4 depicts an overview of an exemplary drug product manufacturing site 150, according to aspects of the present disclosure. In the embodiment of FIG. 4, the site 150 may include the a first group of one or more containers 102 dedicated to drug substance production 124 as well as the second group of one or more containers 102 dedicated to drug product production 126. Each of the first and second groups 124, 126 may include six containers 102, with three of the six containers of each group housing HVAC equipment (for example on the first level or on the second level), as well as three of the six containers housing process modules, as shown in FIG. 3 (i.e., the first group three containers 124 housing the DNA transcription module 128, the first purification module 130, and the first bioburden reduction module 132, and the second group of three containers 126 including the LNP formation module 134, the second purification module 136, and the second bioburden reduction module 138). In some embodiments, the site 150 may include different numbers of containers, and may include other modules as discussed herein (for example, a quality control module 140, a fill and finish module 142, and other modules as described herein). In some embodiments, the first and second groups 124, 126 may encompass a total area (or footprint (for example, a site footprint)) of about 800 m² or less, and may be able to produce approximately 50 million doses of vaccine per year.

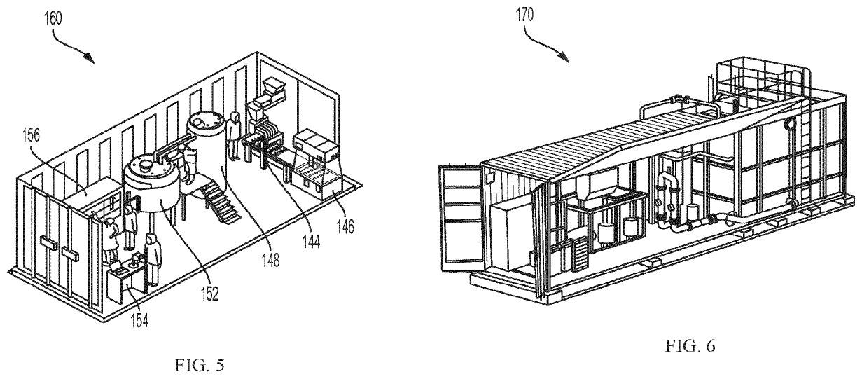

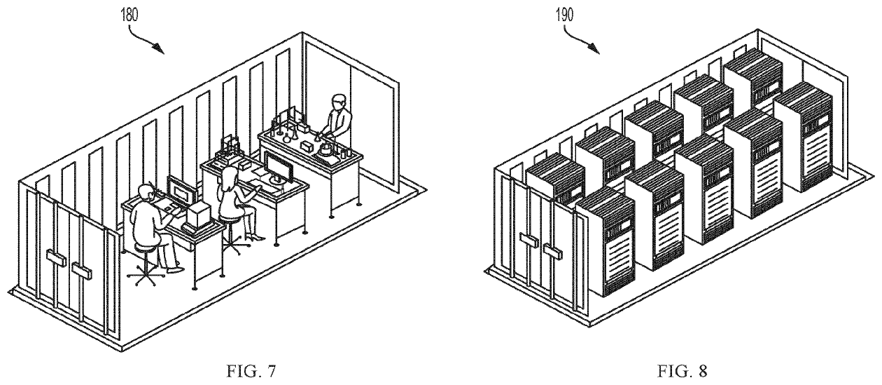

FIGS. 5 and 6 depict an overview of an exemplary drug product manufacturing site and/or module 160, according to aspects of the present disclosure. In the embodiment of FIG. 5, the module 160 may be or include any of the individual modules 128, 130, 132, 124, 126, 128, 140, and/or 142 including combinations thereof, as shown in FIG. 3, and as described herein. Referring to FIGS. 5 and 6, in some embodiments, the module 160 may include all of the associated equipment required for drug substance and/or drug product production in a single container (for example, for use in medium-scale, small-scale, and/or micro-scale production applications). For example, module 160 may be installed at a hospital or other location where individualize treatment and/or therapies are required in a time-sensitive setting, and on a relatively small scale. Module 160 may include equipment 144 for receiving and/or processing drug substance (and/or other process input supplies), tanks 148 and/or mixing equipment 148 (for example, for LNP (i.e., RNA-LNP production)) filtration equipment 152, process control equipment 154, fill and finish equipment 146, and/or storage or refrigeration equipment 156. In the context of larger scale production, certain equipment, such as mixing and filtration equipment, is only commercially available at fixed sizes, thereby partially dictating the size at which drug production can occur. At smaller scales, there may be vastly greater availability of equipment in increment sizes, meaning that the entire drug production (include drug substance and drug product manufacturing) can feasibly fit within a single shipping container 102. In addition, certain process steps or modules such as freezing and/or warehousing may not be required when the drug product is being delivered to (i.e., administered to) the patient as soon as it is ready. In some embodiments, module 160 may be used in connection with treating a single individual with individualized needs. In some embodiments, module 160 may be used to manufacture drug products for a group of individuals affected by the same ailment (for example, a localized viral outbreak or viral strain).

FIG. 7 depicts an overview of an exemplary drug product quality control site and/or module 180, according to aspects of the present disclosure. In some embodiments, a quality control assessment involves an assessment of one or more parameters indicative of product performance and/or quality control.

FIG. 8 depicts an overview of an exemplary drug product warehouse site and/or module 190, according to aspects of the present disclosure.

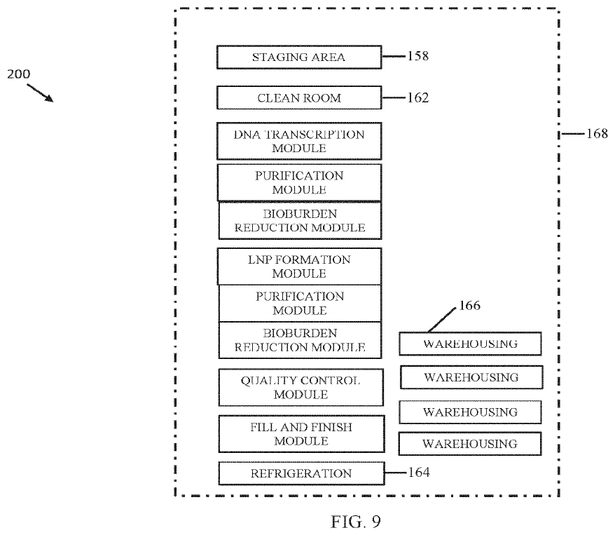

FIG. 9 depicts an overview of an exemplary drug product manufacturing site 200, according to aspects of the present disclosure. In the embodiment of FIG. 9, the site 200 may include about 15 modules or containers 102. In some embodiments, the site 200 may include from about 10 modules to about 20 modules or containers 102. The site 200 may include various numbers of each type of container 102 other than what is shown in FIG. 9. The site 200 may also include other types of modules or containers 102 (for example, HVAC modules), and in some embodiments may not include each and every type of module or container 102 illustrated in FIG. 9. The site 200 may include each of the modules or containers 102 shown in FIG. 3, as well as one or more staging area containers 158 (for example, for receiving shipments and/or visitors to the site 200). The site 200 may also include a clean room module 162 to allow personnel to change into clean working gowns and/or clothing. The site 200 may include one or more refrigeration modules 164 for freezing drug products as well as one or more warehousing modules 166 for storing drug products. The site 200 may be contained within a site boundary 168 that may include, for example, a giant tent, a hangar (for example, an inflatable hangar), and/or other types of temporary or permanent structures, fences, warehouses and/or buildings.

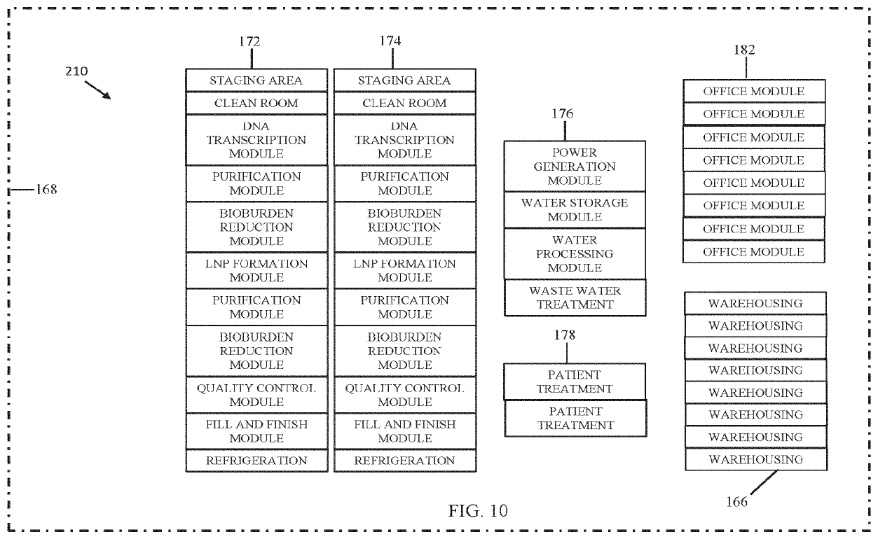

FIG. 10 depicts an overview of an exemplary drug product manufacturing site 210, according to aspects of the present disclosure. In the embodiment of FIG. 10, the site 210 may include about 40 modules or containers 102. In some embodiments, the site 210 may include from about 30 modules to about 50 modules or containers 102, or from about 10 to about 60 modules, and/or more than 60 modules. The site 210 may include various numbers of each type of container 102 other than what is shown in FIG. 10. The site 210 may also include other types of modules or containers 102 (for example, HVAC modules), and in some embodiments may not include each and every type of module or container 102 illustrated in FIG. 10. The site 210 may include each of the modules or containers 102 shown in FIG. 9, as well as a first drug production facility 172 and a second drug production facility 174, both located within the site boundary 168. The first and second drug production facilities 172, 174 may be used to produce the same drug products and/or different products, depending on the need. The site 210 may include a number of utilities 176 including power generation, water storage, water processing, and/or waste water treatment. The site 210 may also include one or more patient treatment areas 178, office areas 182, and/or warehousing modules 166.

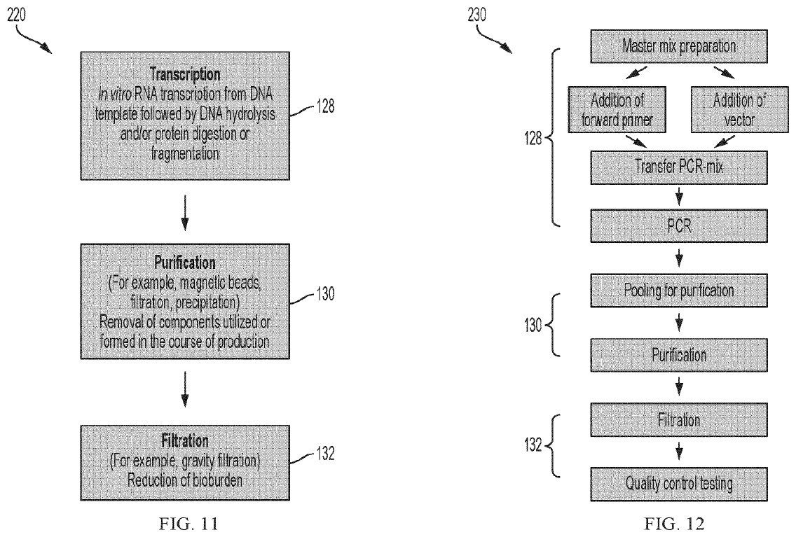

FIG. 11 illustrates an overview of exemplary manufacturing process 220 for a pharmaceutical-grade composition comprising RNA, according to aspects of the present disclosure. The process 220 may include the DNA transcription module 128, the first purification module 130, and the first bioburden reduction (or filtration) module 132, as previously described herein. In the embodiment of FIG. 11, the process 220 includes an exemplary manufacturing process for pharmaceutical-grade RNA comprising an in vitro RNA transcription followed by removal of components utilized or formed in the course of production by a purification process, and filtration to reduce bioburden (e.g., as illustrated in FIG. 11). Optional in-process controls may also be completed depending on whether a hold step is performed.

FIG. 12 illustrates an overview of exemplary DNA template manufacture process 230 via a PCR-based process, according to aspects of the present disclosure. In the embodiment of FIG. 12, the process 230 includes an exemplary manufacturing process of a DNA template via a PCR-based process including the DNA transcription module 128, the first purification module 130, and the first bioburden reduction (or filtration) module 132, as described herein. Initially, a master mix preparation may be made. Subsequently, forward primer and vector may be added. The PCR-mix may be transferred into a reagent reservoir and a PCR plate may be filled. A PCR may be completed, the PCR comprising an initial denaturation, a denaturation step, an annealing step, a final extension step for 20-30 (e.g., 20, 21, 22, 23, 24, 25, 26, 27, 28, 29, or 30) cycles and a hold step. The PCR products may be pooled and purified. Subsequently, the purified, pooled PCR product may be filtered and quality control tested. Accordingly, the purification module 130 may include systems and/or equipment for the addition of elution and/or dilution buffer. In some embodiments, RNA that is bound on magnetic beads is subsequently eluted (e.g., after wash steps) by addition of an elution buffer. In some embodiments, an elution buffer comprises a chelating agent to complex and thus remove residual divalent ions (e.g., magnesium and/or calcium ions) that may be added during RNA synthesis processes. In some embodiments, an elution buffer may comprise EDTA (for example, 18 mM EDTA at a pH of 7). While a skilled artisan will be able to select an appropriate buffer for elution, in some embodiments, an elution buffer may comprise HEPES buffer (for example, 18 mM HEPES at a pH of 7). In some embodiments, an elution buffer is a buffer selected for use in a pharmaceutical-grade composition comprising RNA. A dilution buffer may also include ammonium sulphate. In some embodiments, a dilution buffer may include 300 mM NaCl. In some embodiments, an RNA solution is mixed with both elution buffer and dilution buffer resulting in a mixture comprising from about 42% to about 54% RNA solution by volume, from about 8% to about 28% elution buffer by volume, and from about 30% to about 38% dilution buffer by volume. FIG. 12 illustrates which portions and/or steps of the process 230 are contained within each module or container.

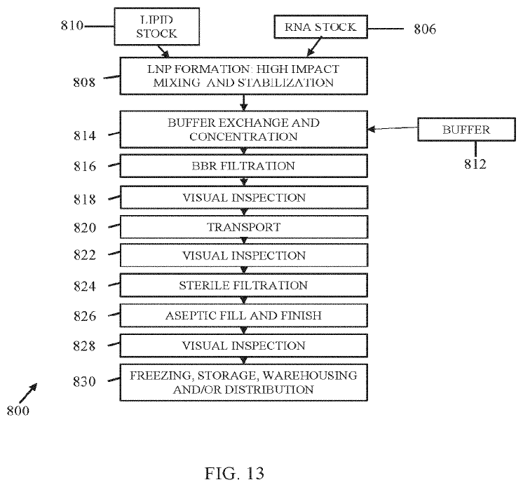

FIG. 13 illustrates an exemplary process 800 for manufacturing LNP compositions. Generally, steps 806, 808, and 810 (as well as equipment associated with those steps, as described herein) will occur and/or be located in the LNP formation module 134. Generally, steps 812 and 814 (as well as equipment associated with those steps, as described herein) will occur and/or be located in the second purification module 136. Generally, step 816 (as well as equipment associated with step 816, as described herein) will occur and/or be located in the second bioburden reduction module 138. Steps following 816 may occur in other modules and/or may occur at other facilities (or not at all). For example, as explained herein, freezing and warehousing may not be required in all embodiments.

Referring still to FIG. 13, as can be seen, the produced compositions are prepared by combining lipids 810 with an aqueous preparation which carries an agent of interest (e.g., an active agent). In many embodiments, the agent of interest is a nucleic acid (e.g., a nucleic acid therapeutic). As depicted in FIG. 13, the nucleic acid is an RNA (e.g., a therapeutic RNA); in many embodiments of this depicted process, a utilized RNA includes at least one open reading frame (ORF) which may, for example, encode a vaccine antigen, a replacement protein, an antibody agent, a cytokine, etc.). In some embodiments a vaccine antigen may be a cancer vaccine antigen or an infectious disease (e.g., viral) antigen. In some embodiments, an RNA encodes a polypeptide that is or comprises a viral antigen such as a coronaviral antigen, such as a spike protein or portion thereof, or relevant variant of the foregoing (e.g., a SARS-CoV-2 spike protein or receptor binding domain thereof, for example, a prefusion stabilized variant thereof), e.g., as is utilized in one or more of mRNA-BNT162a1, mRNA-BNT162b1, mRNA-BNT162b2, mRNA-BNT-162c1, mRNA-1273, CVnCov, CVnCoV2, etc.). In certain embodiments exemplified herein, utilized was an RNA of BNT162b2.

In some embodiments of the process depicted in FIG. 13, the RNA is prepared by in vitro transcription (e.g., of a DNA template which may, for example be a linear template such as a linearized plasmid or an amplicon).

Referring to FIG. 13 and the exemplary process that it depicts, at step 808, the process 800 may include LNP formation by adding lipids 810 to an RNA solution 806, as well as high impact mixing (for example, via impingement jet mixing), and stabilization. Typically, the RNA solution is an aqueous solution.

In many embodiments, the lipids 810 may include one or more of a cationically ionizable (sometimes referred to as “cationic” for simplicity) lipid, a phospholipid, a PEG-lipid, a sterol (e.g., a cholesterol) and an appropriate solvent (e.g., ethanol).

In some embodiments, LNP formation may be performed in presence of a buffer (e.g., a citrate buffer) 812. In some embodiments, the buffer (e.g., a citrate buffer) 812 may be present in the RNA solution 806 prior to mixing with the lipids 810 (for example, via in-line dilution of the water-diluted RNA with the buffer (e.g., citrate buffer) 812 to form the aqueous solution of RNA 806). Stated otherwise, buffer (e.g., citrate buffer) 812 may be added to the RNA solution prior to mixing with the lipid solution 810. In some embodiments, the buffer (e.g., citrate buffer) 812 may also or alternatively be added to the mixture resulting from combining the lipid solution with the aqueous solution 806 (which, as depicted in FIG. 13, is an RNA solution but could, in some embodiments, carry a different agent). In some embodiments, the buffer (e.g., citrate buffer) 812 may include citric acid (monohydrate sodium citrate) and/or sodium hydroxide.

According to embodiments described herein, step 808 (LNP formation) includes forming a first RNA-LNP preparation that includes LNP-encapsulated RNA. LNP formation 808 may include the adjusting of one or more process temperatures, process flow rates, and/or ratios of the buffers, solutions and/or suspensions. LNP formation may include independently flowing each of the aqueous solution and lipids 810 (for example, in a lipid solution) into a mixing unit. Each of the aqueous RNA solution 806 and lipid solution 810 may flow into the mixing unit under laminar flow conditions.

Still referring to FIG. 13, at step 814, the process 800 may include buffer exchange and concentration of the first RNA-LNP preparation to form a second RNA-LNP preparation. The buffer exchange and concentration step 814 may be conducted with process parameters including, for example, a feed flow rate, for example within a range of 18 to 50 liter/min (LPM), a trans-membrane pressure (TMP), for example lower than 1200 mbar, a retentate pressure, for example within a range of 130 to 230 mbar, and a permeate pressure, for example within a range of 10 to 70 mbar.

In some embodiments, buffer exchange 814 of the first RNA-LNP preparation and concentrating the first RNA-LNP preparation are performed in alternating steps. In one or more embodiments, a TRIS (i.e., tris(hydroxymethyl)aminomethane) buffer may be used. In some embodiments, the buffer exchange 814 is conducted via diafiltration and the concentration is conducted via ultrafiltration. In some embodiments, the diafiltration and/or the ultrafiltration are conducted via tangential flow filtration (TFF) (for example, in a tangential flow filtration unit and/or TFF skid). In some embodiments, the tangential flow filtration is conducted using one or more jejunostomy tubes and/or one or more dip tubes. During the tangential flow filtration, a retentate may be recirculated to a feed tank using a dip tube comprising a first end submerged into filtration feed liquid in the feed tank. Prior to the buffer exchange and concentration steps, a filtration system for tangential flow filtration may be filled with a buffer.

Referring still to FIG. 13, the buffer exchange and concentration step 814 may include at least two buffer exchanges conducted via diafiltration alternating with at least two concentrations conducted via ultrafiltration. During buffer exchange and concentration 814, process temperatures may be maintained within a desired temperature range (for example, at or below about 25 degrees C., or from about 2 degrees C. to about 25 degrees C., or from about 15 degrees C. to about 25 degrees C.). During buffer exchange and concentration 814, pH may be continuously monitored (and may be maintained in a target range (for example, from about 7.0 to about 7.5, or from about 7.1 to about 7.3)) and shear may be maintained, for example in a range from about 2000 s\hat{ }−1 to about 6000 s\hat{ }−1, or from about 3000 s\hat{ }−1 to about 5000 s\hat{ }−1, or at about 4000 s\hat{ }−1 (+/−1%, 5%, and/or 10%). Following buffer exchange and concentration 814, a recovery flush may be performed, during which time shear may be reduced to under about 2000 s\hat{ }−1 (for example, under about 1500 s\hat{ }−1, or under about 1000 s\hat{ }−1). In some embodiments, following buffer exchange 814, the pH may be maintained within a range from about 7.3 to about 7.5, for example following ultrafiltration and/or diafiltration.

In some embodiments, during buffer exchange and/or concentration 814, the pH of the first RNA-LNP preparation may be maintained at a pH that is higher than that of the cationic lipid (i.e., the cationic lipid in the lipid solution). Without wishing to be bound by any particular theory, it is proposed that doing so may reduce foaming of the liquid nanoparticles.

In some embodiments, the first and/or second RNA-LNP preparation(s) may be sterilized. In some embodiments, a relevant produced formulation may be a product for further manipulation, processing, packaging, and/or shipping. In some embodiments, a produced formulation may be or comprise a drug product formulation, e.g., for administration to humans.

In some embodiments, one or more sterilization steps may be performed by sterile filtration; in some embodiments, sterile (or other) filtration may be conducted at a target pressure with substantially no pressure building up during the filtration process, for example at about 1.03 bar (or from about 1.02 bar to about 1.04 bar, from about 1.01 bar to about 1.05 bar, or from about 1.00 bar to about 1.1 bar).

In some embodiments, a utilized mixing unit may include one or more impingement jet mixing skids. Prior to mixing, the impingement jet mixing skids may be vented and/or flooded. Mixing of the aqueous and lipid solutions may be performed within boundaries of the mixing unit and/or one or more impingement jet mixing skids. In some embodiments, prior to mixing, the aqueous solution does not contact the lipid solution. In some embodiments, the flow rate ratio into the mixing unit of the aqueous solution to the lipid solution is about 3:1. In some embodiments, the mixing speed may be adapted adjusted to meet process performance requirements. For example, one or more mixing processes may include increasing the mixing speed gradually until a slight vortex has formed (for example, the mixing speed at or slightly above the point at which a visible vortex has formed), but below the mixing speed at which foam begins to form.

Still referring to FIG. 13, the system (for example, the impingement jet mixing skids, the TFF system (i.e., the tangential flow filtration unit), and/or components thereof) may be assessed at one or more time points (e.g., monitored over time, e.g., periodically or continuously) to ensure conformity with process monitoring requirements. In the event that a monitored parameter exceeds a process limit in the aqueous solution, the lipid solution, the first RNA-LNP preparation, the second RNA-LNP preparation, the mixing unit, and/or tubing providing the aqueous solution of RNA, the lipid solution, the first RNA-LNP preparation, and/or the second RNA-LNP preparation, an alert or notification may be sent indicating that there has been an exceedance somewhere in the system.

In some embodiments, the aqueous solution and/or the lipid solution may be flowed into the mixing unit through one or more inlets disposed at a bottom portion of the mixing unit, and the resulting first RNA-LNP preparation may be released from the mixing unit through one or more outlets disposed at a top portion of the mixing unit. In some embodiments, the mixing may be performed with a submerged mixer. In some embodiments, foam may be generated during and/or after formation of the LNP-encapsulated RNA, and may be subsequently removed from the RNA-LNP preparation (for example, the foam may be removed from the first and/or second RNA-LNP preparation).

Referring still to FIG. 13, following buffer exchange 814 and concentration, the process 800 may include 0.2 μm filtration and/or the addition of sucrose and PBS for compounding. Following compounding, the process 800 may include bioburden reduction filtration (BBR) 816 following the buffer exchange and concentration 814. Bioburden reduction filtration 816 may include filtering with 0.2 μm pore size (or for example, about a 0.22 μm pore size) or smaller filter. Bioburden reduction filtration 816 may also include using other pore sizes (for example, 0.45 μm pore size) as described herein. According to the present embodiments, 0.2 μm pore size filtering may also occur on each of the lipid solution and the aqueous RNA solution prior to mixing, on the first RNA-LNP preparation, and/or on the second RNA-LNP preparation. Bioburden reduction filtration 816 may also include filtering the post TFF-LNP suspension through a particulate reduction filter prior to filtering the suspension through (for example) the 0.2 μm pore size and/or 0.22 μm pore size bioburden reduction filter. In some embodiments, bioburden reduction filtration 816 may also include performing a filter recovery flush. In some embodiments, the bioburden reduction steps described herein may include in-process controls to ensure that no greater than about 50 cfu/100 mL (for example, no greater than about 40 cfu/100 mL, no greater than about 30 cfu/100 mL, no greater than about 20 cfu/100 mL, no greater than about 10 cfu/100 mL, and/or no greater than about 5 cfu/100 mL) are present in the flow stream exiting the bioburden reduction filtration step 816. In some embodiments, the system 10 includes one or more automated in-process monitoring and control sensors for measuring bacteria count (i.e., colony forming units). The automated in-process monitoring and control sensors may include one or more spectrophotometers, dip testers, and/or optical sensors configured to measure optical density within a wavelength spectrum from about 500 nm to about 800 nm (for example, from about 550 nm to about 700 nm, or from about 575 nm to about 650 nm, or from about 600 nm to about 630 nm).

Still referring to FIG. 13, following bioburden reduction filtration 816, the process 800 may include filling transport bags (for example, Flexsafe® bags) with the filtered second RNA-LNP preparation, and performing a visual inspection 818 of the transport bags. In some embodiments, transport bags may be, for example 12 L bags, 50 L bags, 100 L bags, and/or other suitable bag sizes (e.g., depending on the batch size of the relevant RNA-LNP preparation), including bags that include a volume between 12 L and 50 L, and/or bags that include a volume between 50 L and 100 L.

In some embodiments, filling transport bags may include filling the bags to a volume in a range from about 30% to about 95%, or from about 40% to about 90%, or from about 50% to about 85%, or from about 60% to about 85% or from about 70% to about 85%, and/or other subranges therebetween of the total bag volume. In some embodiments, prior to filling, the bags may be evacuated, unfilled, and/or otherwise uninflated.

Filled bags may be stored and/or shipped at a temperature in a range from about 1 degree C. to about 15 degrees C. (for example, at about 2 degrees C. to about 10 degrees C., or from about 2 degrees C. to about 8 degrees C.), or alternatively may be frozen to a temperature of about −70 degrees C. (for example, in a range from about −60 degrees C. to about −80 degrees C.). Prior to shipment, the bags may be secured in or on racks and/or within or on any other suitable shelving or storage system so as to minimize movement, rupturing, and/or disruption of the bags during the transport to a fill and finish site. For example, transport bags may be stacked in a specific manner using a stacking system on pallets that include shock absorbers. During transport 820 and/or in preparation for transport 820, as well as following transport, nitrogen with a positive pressure (for example, from about 1-2 bars) may be maintained in and around the environment in which the bags are kept and/or transported.

Referring still to FIG. 13, after arriving at a fill and finish site, sterile filtration 824 may be performed (i.e., the second RNA-LNP preparation). In some embodiments, such sterile filtration 824 may be performed after the preparation has been removed from the transport bags, but prior to being disposed within a collection vessel, reservoir, and/or fill tank. In some embodiments, the material (i.e., the filtered preparation) may then be dispersed from the collection vessel, reservoir, and/or fill tank during aseptic fill and finish 826 (for example, to aseptically fill glass vessels with the drug product). The filled glass vessels, at step 830 of the process 800, may then be frozen, stored, warehoused and/or distributed, for example, to health care administration sites. Alternatively, in some embodiments, filled glass vessels may be subjected to lyophilization or other drying process, so that drug product is transported and/or stored in a dry state (e.g., for subsequent resuspension).

Still referring to FIG. 13, in some embodiments, the fill and finish facility may be located in the same location as the LNP production facility, in which case fill and finish may be performed directly using Point of Fill filtration equipment (in which case the transport 820, bag filling and sealing, and one or more of the visual inspection steps 818, 822, 828 may not be required. In yet other embodiments, the process 800 may include multiple transport steps 820, as well as additional visual inspection steps 818, 822, 828 if the various steps of the process 800 are performed at additional and/or other facilities (or alternatively, if transport is required within a single facility).

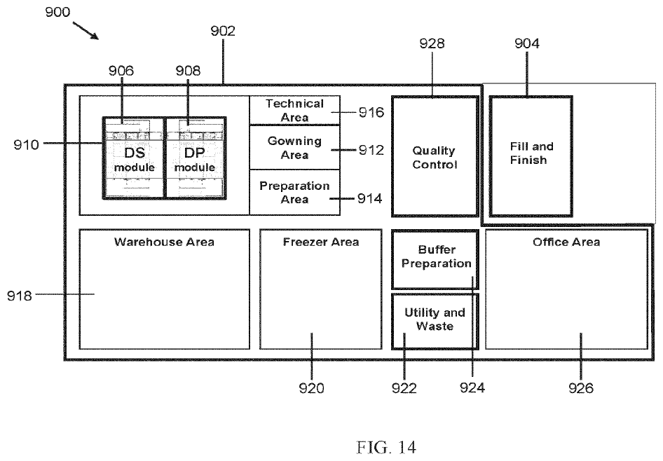

FIG. 14 depicts an overview of an exemplary drug product manufacturing site 900, according to aspects of the present disclosure. In the embodiment of FIG. 14, the site 900 may include a site boundary 902 defining the various areas located at the site 900. In some embodiments, the fill and finish process 904 may be performed at a different location (or may occur on site 900). The site 900 may encompass a total secured ground area of 6,000 m² to 8,000 m², or from about 5,000 m² to about 10,000 m², with a total of about 3,000 m² to 6,000 m², or from about 4,000 m² to about 5,000 m² total sheltered area (that is, total building area). The site 900 may include a drug substance module 906 and a drug product module 908, both enclosed within a clean area boundary 910. Each of the drug substance and product modules 906, 908 may be arranged in a 6-container configuration similar to the arrangements shown in FIG. 4 (i.e., similar to drug substance module 124 and drug production module 126). The drug substance module 906 and the drug product module 908 may encompass a total area of about 800 m², or from about 500 m² to about 1,000 m². Adjacent to the drug substance module 906 and the drug product module 908, the site may include a gowning area 912 and a preparation area 914, which in some embodiments may encompass a combined area of about 300 m². A technical area 916 for electronics, utilities, facility control equipment, as well as other equipment may be located adjacent the gowning area 912.

Referring still to FIG. 14, the site 900 may include a warehouse area 918, a freezer area 920, a buffer preparation area 924, a utility and waste area 922, an office area 926, and a quality control area 928. The warehouse area 918 may encompass a total area of of about 600 m², or from about 500 m² to about 800 m², and may be configured to include inbound and/or outbound logistics to accommodate 1,000 palette positions (for example, each with a standard pallet size of about 1000 mm×1200 mm or about 800 mm×1200 mm). The freezer area 920 may include at least three (3) climate zones, with a first zone maintaining temperatures at or around −70° C. (for example, from about −60° C. to about −80° C.), with a second zone maintaining temperatures at or around −20° C. (for example, from about −10° C. to about −30° C.), and with a third zone maintaining temperatures in a range from about about 2° C. to about 8° C.). In some embodiments, each of the buffer preparation area 924, the utility and waste area 922, the office area 926, the quality control area 928, and/or the fill and finish area 904 may be provided (and/or initially constructed) by the local country and/or region where the site is located, rather than being shipped to the site in one or more standard shipping containers. In some embodiments, the quality control area 928 may encompass an area of about 400 m², the buffer preparation area 924 may encompass an area of about 200 m², the utilities and waste area 922 may encompass an area of about 200 m², the fill and finish area 904 may encompass an area of about 400 m², and the office area 926 may encompass an area of about 800 m². The site 900 may also include a security gate/guard house and/or other suitable structures. In some embodiments, the site may include a second drug product module 908, which would serve to increase the production capacity (for example, to a number greater than 50 million doses per year such as about 80-100 million doses per year), and may add about 300 m² to the site footprint.

Still referring to FIG. 14, the drug substance module (or area) 906 and the drug production module (or area) 908 may be housed in a building with a ceiling height of at least 8 m, with a point load capacity of at least 9,000 kg, and with gates and/or doors with a width of at least 4 m and a height of at least 7 m. The warehouse area 918 (as well as other areas such as the quality control area 928, the buffer preparation area 924, the utility and waste area 922, the fill and finish area 904, the drug substance module 906 and the drug product module 908) may be maintained at a controlled humidity and at a temperature from about 15° C. to about 25° C. The freezer area 920 may include space for about 10-20 freezers as well as about 5 to about 10 refrigerators. The utility and waste area 922 may provide a normal power supply in a range of about 2,000 kW to about 4,000 kW (for example, about 3,000 kW), and may include an uninterrupted power supply of at least 250 KW (for example, from about 200 kW to about 300 kW), to supply power for the entire site. The drug substance and drug product modules 906, 908 may require a total (i.e., combined) normal power of about 300 KW (for example, in a range from about 200 kW to about 400 kw) with an uninterrupted power requirement of about 70 KW (for example, in a range from about 60 kW to about 80 kW, or form about 50 kW to about 100 KW). The utility and waste area 922 may also include a drinking water supply capable of delivering a volume of 4 m³/hr of drinking water to the site. The utility and waste area 922 may also include provisions for providing network connections to the site 900, as well as compressed air, nitrogen, carbon dioxide, and/or oxygen (for example, supplied in bottles). The site 900 may also include various other components, layouts, and arrangements of modules other than what is shown in FIG. 14.

Referring still to FIG. 14, the fill and finish module 904 (or area) may include the necessary equipment for carrying out the following process steps: pooling of liquids or frozen bulk material (for example in grade C conditions); sterile filtration into one or more asceptic filling lines under grade A conditions (for example, via Isolator Technology and/or RABS); capping and crimping of (for example) 2R ISO 8362-1 standard vials up to (for example) 2.25 mL; performing manual, semi-automated, and/or fully automated visual inspection of the filled vials; serialization and labelling of the vials; freezing and storage of the filled vials; and packing and shipping under dry ice. The fill and finish module 904 (or area) may include the necessary equipment to handle about 10 million vials (the equivalent of 50 million doses) annually. In preferred embodiments, the fill and finish module 904 meets one or more standards (for example, the cGMP standard and/or Process Control systems such as Siemens PCS7). In some embodiments, the fill and finish area 904 is located on site 900, while in some embodiments, the fill and finish area 904 is located off site.

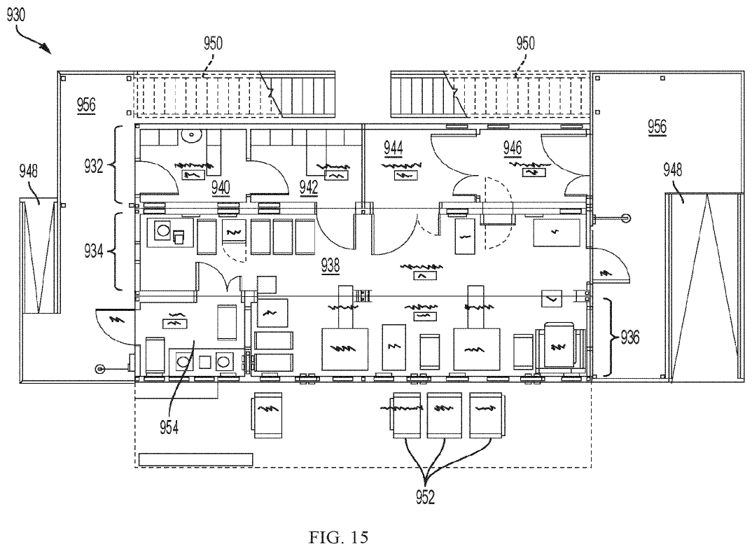

FIG. 15 depicts an overview of an exemplary drug substance module 906 and/or drug product module 908, according to aspects of the present disclosure. The illustration of FIG. 15 shows a possible layout that could be used for both the drug substance module 906 as well as the drug product module 908. The drug substance module 930 and/or drug product module 930 may include 3 containers 932, 934, 936 on a first level (for example an upper level or lower level) of a production facility. The embodiment of FIG. 15 is similar in layout to the 6-container drug substance module 124 and/or drug product module 126 shown in FIG. 4. As such, FIG. 15 may include an additional 3 containers housing HVAC equipment (for example, on a level below or above the level shown in FIG. 15. The first container 932 may be used primarily for staging and for ensuring an airlock is maintained, while the second and third containers may be used primarily to house the operation suite (i.e., for manufacturing drug substance and/or drug product). In a first end, the first container 932 may house a personnel entry section 940 adjacent a personnel airlock area 942. Within a second end, the first container 932 may house a material entry area 946 adjacent to a material airlock area 944. Upon entry within the first container 932, personnel and/or materials must travel through the respective personnel and/or material airlock sections 942, 944, in order for ambient and/or environmental air to be flushed out (for example, via vacuum pumps and/or blowers in cooperation with sealed doors and entryways) to ensure no impurities enter the operational suite 938 when personnel and/or materials enter. In the course of normal operations, personnel and materials should only enter the operation suite 938 through the respective airlocks. The drug substance module 906 and/or drug product module 908 may include manufacturing and/or production equipment within the operation suite 938, as described in the present disclosure. The location of equipment, walls, doors, and/or other structures may be adjusted from what is shown in FIG. 15 as needed to optimize a given production process.

Referring still to FIG. 15, the drug substance module 906 and/or drug product module 908 may include an APEX or hazardous equipment area 954 for housing any materials and/or equipment that is electrified, pressurized, heated and/or otherwise presents a risk of fire, combustion, and/or explosions (for example, equipment falling within the scope of the ATEX 114 “equipment” Directive 2014/34/EU and/or the ATEX 137 “workplace” Directive 1999/92/EC). The hazardous equipment area 954 may also be maintained as a clean room to reduce the likelihood that combustible material and/or debris will enter the hazardous equipment area 954. In some embodiments, the hazardous equipment area 954 (or “ATEX room”) may be used to house one or more drug product components or sub-components used for lipid preparation, which may require the use of ethanol, which may (along with disinfectants) be stored in one or more safety cabinets within the ATEX room 954. The drug substance module 906 and/or drug product module 908 may also include one or more stairways 950 (for example, leading up or down to the HVAC level) as well as one or more ramps 948 to facilitate the ease with which materials (and/or personnel) may be transported to one or more platforms 956 leading to the personnel and material entryways 940, 946. The drug substance module 906 and/or drug product module 908 may include additional external equipment 952 such as transformers, tanks, HVAC equipment, and other necessary components located outside of the containers 932, 934, 936, but in fluid, electrical, thermal, and/or operative communication with components in the interior of the containers 932, 934, 936.

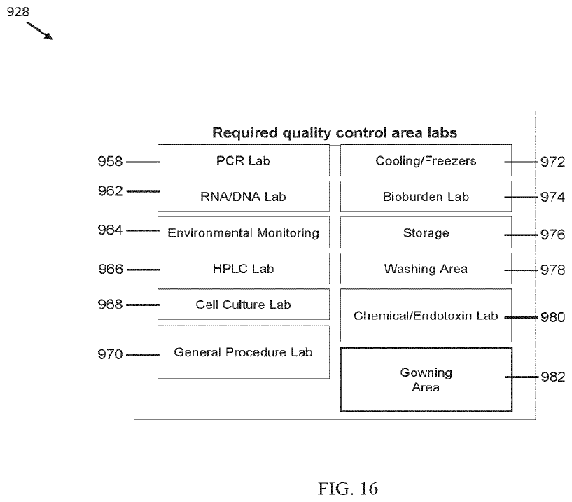

FIG. 16 depicts an overview of an exemplary quality control module 928, according to aspects of the present disclosure. The quality control module 928 may include a PCR lab 958, an RNA/DNA lab 962, an environmental monitoring console 964, a high performance liquid chromatography (HPLC) lab 966, a cell culture lab 968, a general procedure lab 970 (for example, for monitoring such parameters as pH, color, sample logistics, and/or storage metrics), freezer monitoring equipment 972, a bioburden lab 974, a quality control storage area 976, a washing area 978, a chemical/endotoxin lab 980, and/or a gowning area 982. The processes by which quality is controlled (for example, within each of the processes and/or labs illustrated in FIG. 16) may be tightly controlled and/or standardized to reduce the introduction of sources of variation into each lab. The quality control module 928 may be configured such that access by staff/personnel to each of the lab areas is possible only through the gowning area to ensure proper sanitizing and disinfecting measures are taken prior to entering the lab areas within the quality control module 928.

In connection with the modular drug production system 100 of the present embodiments, various personnel and staffing needs must be met in order to ensure consistent and satisfactory operations of the modular drug production system 100. For example, in connection with the drug substance module 906 and/or the drug product module 908, qualified operators/personnel are needed for: operating bioreactors and ensuring laminar flow, monitoring purification protocols, monitoring formulation and filtration processes, process support protocols, as well as production and/or automation engineers, scientists, and/or technicians. In connection with the quality control module 928, scientists and/or technicians with laboratory experience are required for performing various tests to ensure quality control is maintained. In connection with the fill and finish module 904, staff is needed for filling operations, optical control, labelling, and packing. In connection with the warehouse area 918, personnel are needed for logistics and warehousing, while the office area 928 requires personnel with a wide range of skills including (but not limited to) subject matter experts, scientists, engineers, automation experts, quality control/quality assurance experts, procurement/supply chain specialists, finance professionals, human resource professionals, training managers, IT administrators, and overall operations coordinators.

The modular drug production system 100 of the present embodiments may be transported to (and further built or developed as needed) at sites that have solid street access, access via train, and/or shipping to allow each of the containers and other equipment and materials to be transported to site. In some embodiments, the modular drug production system 100 may be transported to (and further built at) existing pharmaceutical installations such that existing laboratory equipment (for example, to be used as a quality control module 928) and/or fill and finish capacity may be utilized. Proximity to universities may also be beneficial to allow staffing needs (for example, including employees/personnel with the requisite technical backgrounds) to more easily be met. In preferred embodiments, sites are located within 1 hour from airports, harbors, and/or train stations to help facilitate logistics and supply chain operations. In some embodiments, the site may be situated in more remote locations, in which cases additional time should be allowed for materials and equipment to arrive at sight, and careful consideration should be taken to ensure that the required qualified personnel will be available to help operate the modular drug production system 100. In addition, and as discussed in this disclosure, the site or location of the modular drug production system 100 installation requires access to power, drinking water, internet/network connections, and must be able to be continuously secured (that is, 24 hours a day, seven days a weeks).

The modular drug production system 100 of the present embodiments may be used in connection with 12.8 gram, 25.6 gram, 38.4 gram, 40 gram and/or other sized production scales. For example, the modular drug production system 100 of the present embodiments may be used in connection with drug product outputs that include 25 L bags of 12-gram batches. Various numbers of containers and types of containers have been described in connection with the embodiments of the present disclosure. It should be appreciated that the modular drug production system 100 of the present embodiments may be used in connection with other types and numbers of containers than those explicitly described here. Standard shipping containers, as described herein, may include a width of about 8 ft. (2.43 m), a height of about 8.5 ft. (2.59 m) and a length from about 20 ft. (6.06 m) to about 40 ft. (12.12 m). Other standard sizes and half sizes and/or large and small sizes may also be used. The modular drug production system 100 of the present embodiments may be used in connection with processes of various production scales. As such, some of the process parameters may vary, while much of the general process flow and equipment will largely remain the same for micro, small, medium, and large-scale drug production. The modular drug production system 100 of the present embodiments has been generally described in connection with RNA-LNP drug production, but may also be used in connection with the production of other drugs.

### Configuration Flexibility—LNPs, Liposomes, and Lipoplexes

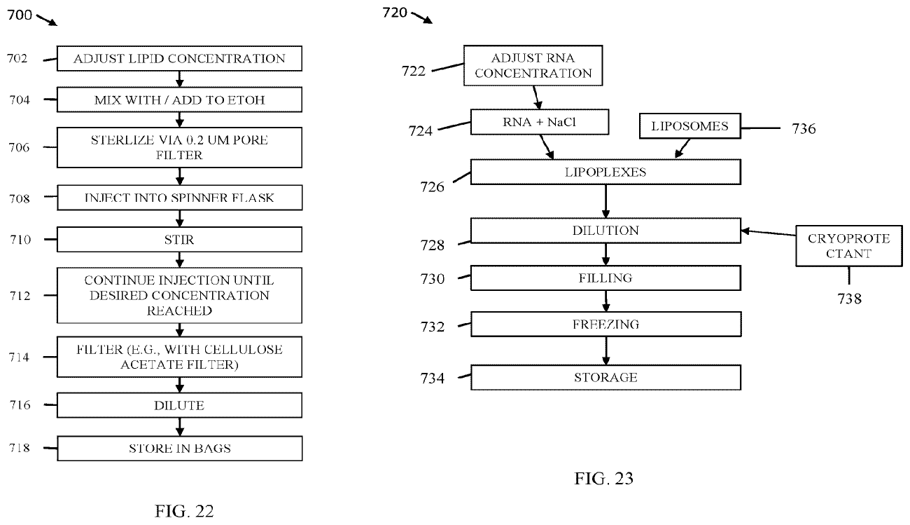

In certain aspects, the present embodiments may include configurable drug manufacturing production facilities (for example, modular facilities) that could be used for research and development, small batch production and even large volume production in a single facility capable of producing mRNA-based products, including those that include lipid nanoparticles (LNPs), liposomes, lipoplexes, and cancer vaccines/therapies, as shown in FIGS. 22-25, and as described herein. In some embodiments, the drug production facility includes a drug substance module for producing RNA solution, and a drug product module in which the drug substance is mixed with lipids to selectively form LNPs, liposomes, and/or lipoplexes, thereby enabling mRNA-based products and therapies in a flexible, modular facility. In some embodiments, the configurable drug manufacturing production facility also includes a fill and finish facility. In some embodiments, the fill and finish facility includes lyophilization capability as well as autoclave capability.

FIG. 22 illustrates a process or method 700 for making liposomes, according to aspects of the present disclosure. At step 702, the method 700 may include adjusting a lipid concentraction. At step 704, the method 700 may include adding the lipid concentration to, and mixing it with, ethanol. At step 706, the method 700 may include sterilizing the mixture via filtration with a 0.2 micrometer pore size (or in some embodiments, a 0.45 micrometer pore size, or in some embodiments, a pore size in a range from about 0.1 micrometers to about 0.5 micrometers). At step 708, the method 700 may include injecting the filtered mixture into a spinner flask. At step 710, the method 700 may include stirring the mixture. At step 712, the method 700 may include continuing injection of the mixture into the spinner flask until a desired concentration is reached. At step 714, the method 700 may include filtering the mixture (for example, with a cellulose acetate filter), thereby forming filtered liposomes. At step 716, the method 700 may include diluting the filtered liposomes. At step 718, the method 700 may include storing the diluted liposomes in bags. In some embodiments, each of the filtration steps as described herein (for example, including 0.2 μm filtration steps and/or steps 132, 706, 816, 824) may include operating with an mRNA mass to filter surface area ratio of about 1.0 mg/cm\hat{ }2 to about 2.0 mg/cm\hat{ }2 (for example, in a range from about 1.2 mg/cm\hat{ }2 to about 1.8 mg/cm\hat{ }2, or in a range from about 1.4 mg/cm\hat{ }2 to about 1.6 mg/cm\hat{ }2, or about 1.5 mg/cm\hat{ }2).

FIG. 23 illustrates a process or method 720 for making lipoplexes, according to aspects of the present disclosure. The method 720 may include using a pre-sterilized single use fluid path, allowing for safe aseptic handling of the materials. First, at step 722, the concentration of the RNA is adjusted to the liposome concentration, and NaCl is added for condensation of the RNA (i.e., at step 724). As a result, in some embodiments, the RNA solution is adjusted to an RNA concentration allowing for mixing of identical volumes of RNA and liposomes. At step 736, liposomes (for example, in a liposome solution) are provided. At step 726, both the RNA and the liposome solution are transferred into large-volume syringes and both syringes are mounted onto a single syringe pump simultaneously driving the two pistons of the syringes, and thereby enabling lipoplex formation. After RNA lipoplex formation, at step 738, a cryoprotectant solution is added and the final concentration of the drug product is adjusted and/or diluted at step 730. In some embodiments, the cryoprotectant 738 is sterile filtered prior to being added to the lipoplex solution. In some embodiments, the method 720 may include sterile filtration (for example, 0.2 μm filtration) following lipoplex formation 726 but prior to addition of the cryoprotectant at step 738. In some embodiments, sterile filtration may occur before dilution 728, while in others, sterile filtration may occur after dilution 728. In some embodiments, the method 720 may include a final filtration step (for example, using a filter with 5 μm pores) following addition of the cryoprotectant to remove any aggregates that may have formed. In some embodiments, the cryoprotectant used at step 738 includes (1) a sucrose buffer in a weight percent range of about 13% to about 18% (for example, from 14-17%, for example, about 15.5%); (2) a HEPES buffer at a concentration of 4.2 mM (for example from about 3.8 mM to about 4.6 mM, or from about 4.0 mM to about 4.4 mM); (3) an EDTA buffer at a concentration of about 1.2 mM (for example, from about 0.8 mM to about 1.6 mM, or from about 1.0 mM to about 1.4 mM); and (4) a pH of about 6.7 (for example, in a range from about 6.3 to about 7.1, or from about 6.5 to about 6.9). After filling of the drug product into glass bottles at step 732, the drug product is frozen as a concentrate at step 732 for storage at step 734 (for example, intermediate storage at site, or in some embodiments, long term storage in a warehouse or storage facility (i.e., at a different site)). In some embodiments, each of steps 702-718 of the liposome formation process 700, as well as steps 722-728 and step 738 of the lipoplex formation process 720 may occur at a temperature in a range from 18° C. to about 25° C. (i.e., at about room temperature).

Referring still to FIG. 23, at step 726, the RNA solution 724 (i.e., drug substance) may be mixed with liposomes 736 at a volume ratio of about 1:1 (plus or minus 5-10%, i.e., at a volume ratio of from 0.95:1 to 1:1.05, or at a volume ratio of from 0.9:1 to 1:1.1), for example, in some embodiments, at a total flow rate of about 100 mL/min to about 800 mL/min, or from about 200 mL/min to about 600 mL/min, or from about 250 mL/min to about 550 mL/min, or from about 300 mL/min to about 500 mL/min, or from about 300 mL/min to about 450 mL/min, or from about 320 mL/min to about 400 mL/min, or from about 350 mL/min to about 450 mL/min. In some embodiments, the liposomes may include L6 liposomes, for example, comprising about 70% by weight phospholipon 100H loaded with sodium hyaluronic acid, about 20% by weight cholesterol, and about 10% by weight stearylamine. In some embodiments, after lipoplexation, the RNA-liposome mixture may be incubated at a temperature in a range from about 18° C. to about 25° C. (i.e., room temperature) for a period of about 10-20 minutes.

A critical quality attribute of RNA lipoplexes is the charge ratio adjusted by the mixing ratio between RNA and liposomes. An automatable and scalable industrial manufacturing process for RNA lipoplexes allowing for efficient control of the mixing ratio may be used. For example, in a process for manufacturing of small scales (≤10 liters), control of the mixing of identical volumes of two aqueous solutions containing liposomes and RNA may be achieved by using a single perfusor pump simultaneously driving two large-volume syringes filled with RNA or liposomes. For equivalent pumping of larger volumes (≥10 liters) pumping systems as pressurized vessels, membrane pumps, gear pumps, magnetic levitation pumps, and/or peristaltic pumps may be used in combination with flow rate sensors with feedback-loop for online-control and real-time adjustment of the flow rate.

For automated RNA lipoplex manufacturing, a static mixing element ensuring efficient mixing of the aqueous solutions containing RNA and liposomes may be required. Commercially available microfluidic mixing elements containing serpentines and embedded structures for enhancement of mixing, as well as prototype mixing elements with comparable architecture were found to block during manufacturing. Therefore, these mixing elements are not suited for automated manufacturing of RNA lipoplexes. Y-type and T-type mixing elements with diameters between 1.2 mm and 50.0 mm were found to be suited for automated manufacturing of RNA lipoplexes.

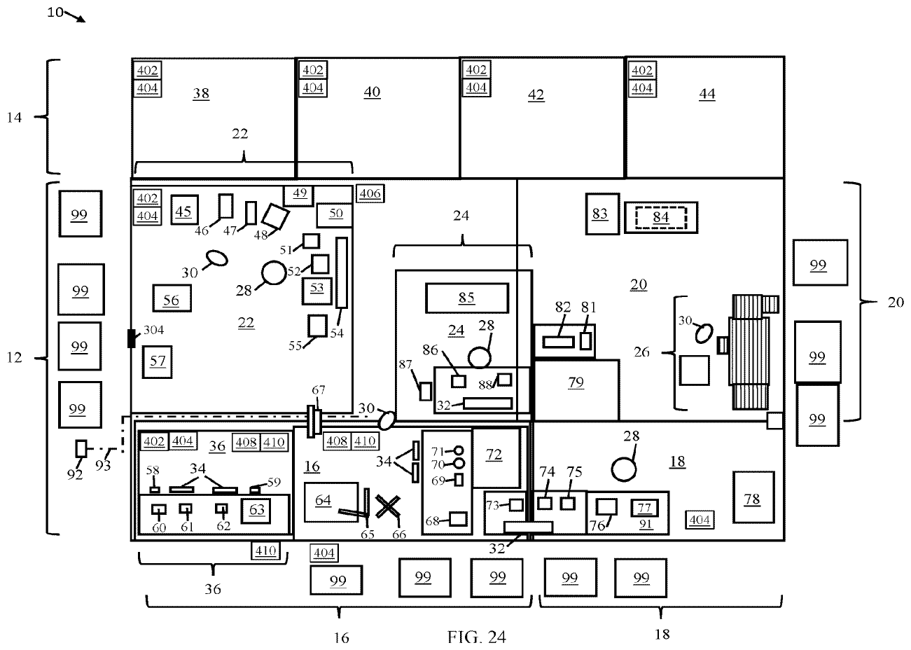

Referring still to FIG. 23, one or more ethanol injection techniques may be used in connection with step 724 and/or step 726 of the method 720. The term “ethanol injection technique” refers to a process in which an ethanol solution comprising lipids is rapidly injected into an aqueous solution through a needle. This action disperses the lipids throughout the solution and promotes lipid structure formation, for example lipid vesicle formation such as liposome formation. Generally, the RNA lipoplex particles described herein are obtainable by adding RNA to a colloidal liposome dispersion. Using the ethanol injection technique, such colloidal liposome dispersion is, in one embodiment, formed as follows: an ethanol solution comprising lipids, such as cationic lipids like DOTMA and additional lipids, is injected into an aqueous solution while the solution is being stirred. In one embodiment, the RNA lipoplex particles described herein are obtainable without a step of extrusion. In some embodiments, the liposome formation method 700 and/or the lipoplex formation method 720 described herein may also include one or more sonication steps (for example, after lipoplex formation at step 726 and before dilution at step 728). In some embodiments, sonication is used during the lipoplex formation step 726 via one or more sonicators (for example, within the lipoplex formation area 20 shown in FIG. 24) which may include an induction-driven impeller or mixer (for example, for creating a vortex, in some embodiments) and/or an ultrasonic bath.

FIG. 24 depicts an overview of an exemplary drug product manufacturing site and/or module 10, according to aspects of the present disclosure. The module 10 illustrated in FIG. 24 may be configured to carry out the liposome and lipoplex manufacturing/formation methods 700, 720 shown in FIGS. 22 and 23, respectively, as well as the LNP formation processes described in connection with FIGS. 11-13. In some embodiments, production and/or formation of lipids and/or liposomes occur offsite while production of lipid nanoparticles and/or lipoplexes occurs on site using lipids and/or liposomes produced offsite. As shown in FIG. 24, the module 10 includes a main production area 12 and a staging or airlock area 14. The main production area 12 may occupy the area of 3 shipping containers (roughly 25 m\hat{ }2 each for a total area of approximately 75 m\hat{ }2, +/−10%) while the staging area 14 may be disposed within a single shipping container (i.e., about 25 m\hat{ }2). In other embodiments, different production and staging areas 12, 14 can be used that are different (for example, larger or smaller) than the respective areas described herein. The main production area 12 may include a drug substance module 22 (i.e., for producing RNA solution) an LNP formation area 16, a lipoplex formation area 20 (including CALF (continuous automated lipoplex formation) equipment 26, an excipient/buffer prep area 18, and/or a shared use area 24. The airlock area 14 may include personnel staging areas 38, 40 including a personnel entry area 38 and a personnel airlock 40, as well as material staging areas 42, 44 including a material inlet area 44 and a material airlock 42. In some embodiments, the CALF equipment 26 may include a peristaltic pump (for example, a 2-roller, 3-roller, 4-roller peristaltic pump) with 2 or more channels to reduce pulsations (i.e., a low pulsation peristaltic pump). The rollers may be angularly offset from one another around an inner circumference of the peristaltic pump such that pulsations are minimized or eliminated. In some embodiments, the CALF equipment may include a rocker mixer to aid in lipoplexation (for example, in connection with step 726 of method 720). In some embodiments, the peristaltic pump may include a dual head. In some embodiments, in connection with lipoplexation 726, the peristaltic pump may operate at an operating flow in a range from about 1 mL/min to about 500 mL/min (for example, from about 25 mL/min to about 300 mL/min, about 35 mL/min to about 200 mL/min, or from about 50 mL/min to about 100 mL/min). In some embodiments, in connection with lipoplexation 726, the peristaltic pump may operate at delta pressure range (i.e., pressure difference between discharge and suction) from about 0.05 bar to about 1 bar, or from about 0.1 bar to about 0.8 bar, or from about 0.2 bar to about 0.6 bar).

Referring still to FIG. 24, the drug substance module 22 may include a TFF skid 45, an IVT control unit 46, an IVT bolus scale 47, an IVT supply unit 48, one or more peristaltic pumps 49, a cleaning trolley/cart 50, pipette equipment 51, a magnetic stirrer 52, an IVT reactor scale 53, a laminar flow bench 54, an IVT raw material scale 55, a thawing area/chamber 56, and/or a chromatograph (for example, a cellulose chromatograph) 57 (or chromatograph unit). In some embodiments, waste from the chromatograph unit 57 may be transferred from an interior of the drug substance/drug product module 10 to one or more waste receptacles (for example, a bagtainer 99) located outside the module 10 via one or more wall pass throughs 304. The LNP formation area 16 may include an ATEX area 36, multiple peristaltic pumps 58, 59, multiple bagtainers 34, an ethanol cannister/mixing unit 60, magnetic stirrers 61, 62 (in some cases including one or more heating and/or cooling jackets), a mix control unit 63, a t-mixer 64, connection pipes 65, an IV pole 66, a charging station 67, an additional magnetic stirrer 68 adjacent to or disposed within a TFF skid 69, collection containers 70, 71, a second set of bagtainers 34 adjacent the collection containers 70, 71, a mix control unit 73, and/or a material processing area 72. In some embodiments, the following items are contained within the ATEX area 36: the multiple peristaltic pumps 58, 59, the multiple bagtainers 34, the ethanol cannister/mixing unit 60, magnetic stirrers 61, 62 (in some cases including one or more heating and/or cooling jackets), and the mix control unit 63. The lipoplex handling area 20 may include continuous automated lipoplex formation (CALF) equipment 26, a mixing unit 79, a biowelder 82, a biosealer 81, a refrigerator 83, and/or a bag tester 84. In some embodiments, the refrigerator 83 may include a combination refrigerator/freezer 83. In some embodiments, the CALF equipment 26 is used to determine a precise weight of the transferred (or added) cryoprotectant (for example, at step 738 of process 720) such that an accurate determination of the ratio of RNA to liposomes may be achieved during lipoplexation (i.e., at step 726 of process 720).

Still referring to FIG. 24, the buffer prep area or module 18 may include a filter holder 32, a magnetic stirrer 74, a scale 75, a buffer mixing unit 78, and/or a buffer prep space 91 including a recirculating hood 91, a pH sensor 76, and a conductivity sensor 77. The shared area 24 may include a charging station 85, a mobile HMI (human-machine interface, i.e., visual display panel with local controls) 87, a magnetic stirrer 86, a scale 88 for fill and finish, and/or a filter holder 32 (i.e., a second filter holder 32 in addition to the filter holder 32 located in the buffer prep area or module 18). The drug production facility/module 10 may include multiple seated workstations 28 and multiple standing workstations 30. The drug production facility/module 10 may also include multiple external handling pods 99 including but not limited to: bagtainers for waste TFF, scales for TFF waste products, scales and bagtainers for waste chromatograph materials, scales and bagtainers for waste permeate and/or drug product waste, air handling units, scales and bagtainers for buffer, filter integrity testers, filter housing, peristaltic pumps, and additional pump skids. The drug production module 10 may also include an oxygen utility (for example, an oxygen supply 92 connected to an oxygen line 93) for supplying oxygen to be used in connection with cancer vaccines and therapies. In some embodiments, the waste containers (i.e., bagtainers and/or other waste vessels or containers) include sensors for determining the volume of the waste contained therein, thereby allowing the system (i.e., an automated system) and/or the operators to be alerted such that mitigating action can be taken to empty and/or replace the full containers.

Referring still to FIG. 24, the drug product manufacturing site and/or module 10 may include multiple sensors including pressure sensors 402, individual and/or combined temperature and humidity sensors 404, hydrogen peroxide (H₂O₂) sensors 406, ethanol sensors 408, and oxygen (O₂) sensors 410. In some embodiments, pressure sensors 402 and individual and/or combined temperature and humidity sensors 404 may be disposed within personnel and material entry and staging areas 38, 40, 42, 44, within the ATEX area 36, and/or within the drug substance area 22. In some embodiments, combined temperature and humidity sensors 404 may also be disposed within the preparation area 18 and/or around the periphery of the drug product manufacturing site and/or module 10. In some embodiments, one or more hydrogen peroxide sensors 406 may be disposed near the shared area 24 and/or outside, but close to the drug substance area 22. In some embodiments, ethanol sensors 408 may be disposed within the ATEX area 36 and within the drug product area 16. In some embodiments, oxygen sensors 408 may be disposed within the ATEX area 36, within the drug product area 16, as well as outside of the ATEX area 36 near the periphery of the drug product manufacturing site and/or module 10.

Referring to FIGS. 22-24, module 10 illustrated in FIG. 24 may be arranged such that it may carry out the formation of LNPs via a two-step process involving the preforming of lipid nanoparticles in a first step, and the addition of an mRNA payload or drug substance to the preformed LNPs during a second step. For example, using the two-step LNP formation process, FIG. 22 can be followed (with the following modifications) to form preformed LNPs. Using the two-step LNP formation process, at step 704 of method 700, a lipid mixture in organic phase is mixed with an acidified aqueous phase (for example, using a T-mixer). Steps 706-718 may then be performed, as described above, to form preformed LNPs (i.e., rather than liposomes). For example, organic solvents may be removed at step 706; dialysis or filtration may occur at step 714; and the preformed LNPs may be optionally frozen or stored at step 718. The preformed LNPs can then be used at step 736 of method 720 (i.e., instead of liposomes) in connection with the remaining steps of method 720 to form mRNA-LNPs via the two step process.

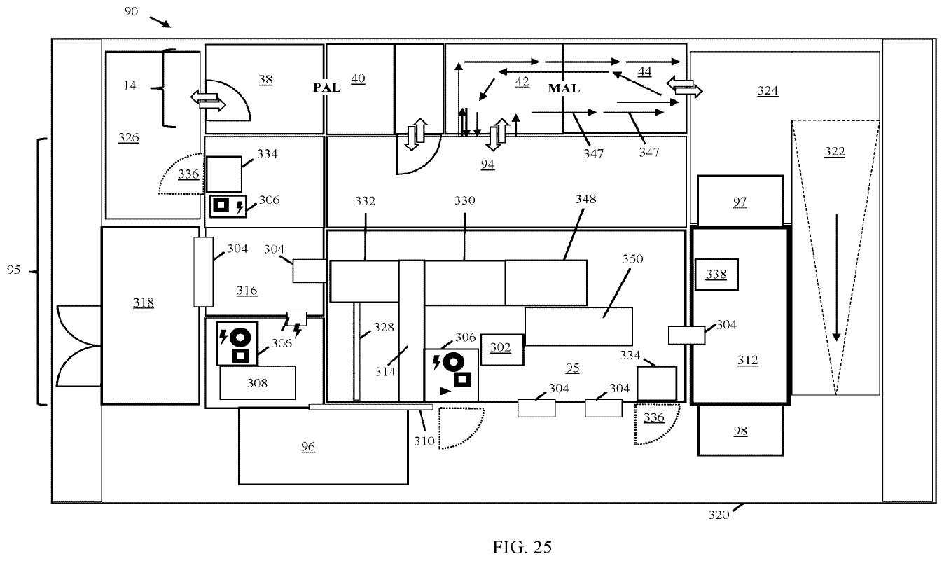

FIG. 25 depicts an overview of an exemplary fill and finish module 90, according to aspects of the present disclosure. In some embodiments, the fill and finish module 90 occupies approximately the same footprint (~100 m\hat{ }2) as the drug production module 10. For example, in some embodiments, about 3 containers worth of area (75 m\hat{ }2) is occupied by the fill and finish workflow 95 and other processing, and about 1 container of space (~25 m\hat{ }2) is occupied by airlocks 14. As shown in FIG. 25, the airlocks 14 may include personnel airlocks (PAL) 38, 40 and material airlocks (MAL) 42, 44 similar to the configuration of FIG. 24 (and detail from FIG. 25 may also apply to FIG. 24, and vice versa). The fill and finish module 90 may also include a clean room area 94 (for example, a Grade C clean room 94), lyophilization equipment 96, one or more autoclaves 97, and machine-washing equipment 98 (for example, in-line machine washing equipment) as shown in FIG. 25. In some embodiments, the lyophilization equipment comprises an Optima lyophilization (freeze-dry) system. In some embodiments, the lyophilization equipment comprises freeze-dry systems made by other manufacturers.

Referring still to FIG. 25, the exemplary fill and finish module 90 may further include space to store drug product 302 and wall pass throughs 304 (i.e., between one portion of the fill and finish module 90 and another, and/or between clean room grade C area 94 and controlled not classified (i.e., “CNC”) area 320 (i.e., area within the shell building that is not within a clean room area 94). The exemplary fill and finish module 90 may further include multiple utilities 306 (as described herein), one or more working areas 308 (i.e., desk/bench 308), a lyophilization opening and/or installation plate 310, a grade C (i.e., clean room grade C) preparation room 312, an air return conduit 314, and a tray loading area, a packing area 318 (i.e., disposed within the CNC area, defined by the CNC boundary 320). The utilities 306 are distributed throughout the fill and finish module 90 in multiple locations and in additional areas to what is illustrated in FIG. 25. The air return conduit 314 may include one or more vents, and may be used to circulate air out of the fill and finish module 90 via a unidirectional flow configuration (that is, air can only flow out of the fill and finish module 90 via the air return conduit 314 (i.e., air cannot flow into the fill and finish module 90 via the air return conduit 314)) in order to maintain aseptic conditions (for example, Grade A air) within the fill and finish module 90. Because one or more pieces of equipment within the fill and finish module 90 may use or be powered by compressed air, as discussed herein, the air return conduit 314 provides the function of allowing an approximately equal amount of air to flow out of the fill and finish module 90. The exemplary fill and finish module 90 may further include a ramp area 322 for loading materials and supplies onto a materials platform area 324 adjacent to the material airlocks (MAL) 42, 44. The exemplary fill and finish module 90 may further include a personnel platform 326 adjacent the personnel airlocks (PAL) 38, 40.

Referring still to FIG. 25, in some embodiments, the exemplary fill and finish module 90 may further include a filling machine 330, as disclosed herein, and a capping and crimping machine 332, for capping and crimping the containers/vials once they have been filled. The exemplary fill and finish module 90 may further include a lyophilization conduit 328 that connects the interior of the fill and finish module 90 to the lyophilization equipment 96 such that, as needed, filled containers may be lyophilized during the process (for example, after being filled via the filling machine 330, and prior to capping and crimping 332). In some embodiments, the air return conduit 314 may be positioned in between and/or adjacent to each of the filling machine 330 and the and the capping and crimping equipment/machine 332. In some embodiments, the exemplary fill and finish module 90 may further include one or more escape doors 336 (i.e., emergency exits 336). In some embodiments, the exemplary fill and finish module 90 may include one or more marked areas 334 including markings on the floor adjacent the escape doors 336 to signify to operators not to put equipment, furniture, supplies, etc. in the marked areas 334 so as to avoid a potential safety hazard that could occur if furniture and/or equipment is obstructing the escape doors 336. In some embodiments, the exemplary fill and finish module 90 may include one or more glove box testers 338 such that operators may use the glove box to aseptically perform testing on drug product samples without risking contamination of the drug product.

Still referring to FIG. 25, the exemplary fill and finish module 90 may further include a debagging machine 350 and a denesting machine 348 that are used prior to drug product (i.e., bulk drug product) entering the filling machine 330. In some embodiments, the debagging machine 350 may include a TUM 9030 model machine (Bausch and Ströbel). In some embodiments, the denesting machine 348 may include a DDM9205 model machine (i.e., VarioSys model from Bausch and Strobel). In some embodiments, the filling machine 330 may include a KSF5105 model machine (VarioSys). In some embodiments, the capping and crimping machine 332 may include a KS1025 model machine (Bausch and Ströbel). In some embodiments, other appropriate models and machines may be used for each of the debagging machine 350, the denesting machine 348, the filling machine 330, and the capping and crimping machine 332. In some embodiments, bulk drug product may be double bagged (i.e., with an inner bag contained within an outer bag). In some embodiments, the debagging machine 350 partially removes the outer bag, but continues to hold the inner bag using the outer bag such that the debagging machine 350 does not make direct contact with the inner bag. In some embodiments, before the inner bag is opened or removed, and while the inner bag is being held within the debagging machine 350 with the outer bag partially removed, the outer periphery of the inner bag is sanitized (for example, via a misting or vaporizing sanitizing process). In some embodiments, Grade A air (i.e., that enters the exemplary fill and finish module 90 via at least an air filtration system that ensures Grade A conditions are maintained) is continuously circulated throughout the exemplary fill and finish module 90). FIG. 25 also illustrates via arrows 347 the path that materials take into and out of the module via the material airlock areas 42, 44.

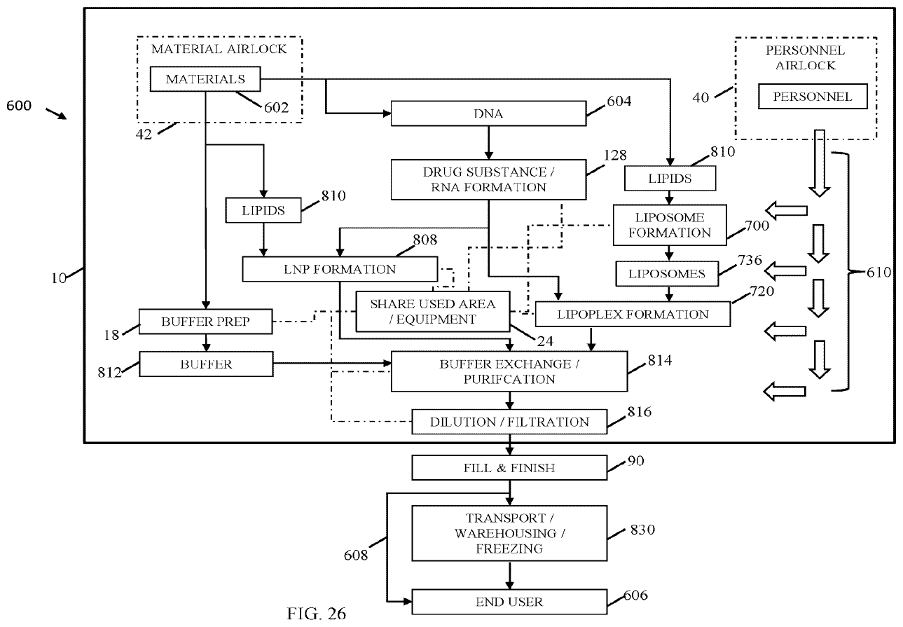

FIG. 26 depicts an overview of a combined lipid nanoparticle (LNP) and lipoplex (LPX) process flow 600, according to aspects of the present disclosure. The combined process flow 600 shows that the drug substance formation processes 220, 230 (illustrated in FIGS. 11 and 12), the LNP formation processes 120, 800 (illustrated in FIGS. 3 and 13), the liposome formation process 720 (shown in FIG. 22), and the lipoplex formation process 720 (shown in FIG. 23) may all be performed within the module 10 illustrated in FIG. 24, using shared resources in a combined process flow 600. For example, materials 602 may enter the module 10 via material airlock 42, and then may take various paths through module 10 depending on which portion(s) of the combined process flow 600 they are following. The materials 602 that enter the module 10 via the material airlock 42 may include lipids 810, DNA stock material 604, ethanol (for example, for use in liposome and LNP formation), buffer ingredients, and/or other materials, as described herein. Similarly, personnel involved in carrying out aspects of the combined process flow 600 (as well as for observing and/or monitoring or taking data on aspects of the process flow 600) may enter the module 10 via the personnel airlock 40, as indicated by arrows 610 shown in FIG. 26.

Referring still to FIG. 26, once materials 602 have entered the module 10, they may be routed to various processes. For example, DNA 604 may be routed to the drug substance/RNA formation sub process flow 128, as described herein in further detail in connection with FIGS. 11 and 12. Lipids 810 may be routed selectively to either an LNP formation module 808 (as described herein in further detail in connection with FIG. 13) and/or to the liposome formation module 700 (as described herein in further detail in connection with FIG. 22). Once formed, liposomes 736 may be used in connection with the lipoplex formation module 720 (as described herein in further detail in connection with FIG. 23). Once formed, RNA solution/drug substance may be routed from the drug substance/RNA formation module 128 to either the LNP formation module 808 and/or the lipoplex formation module 720. Each of the LNP formation module 808 and the lipoplex formation module 720 may be fluidly coupled upstream of a buffer exchange and/or purification module 814 (described herein in more detail in connection with FIG. 13). Accordingly, in some embodiments, the module 10 includes an LNP formation module 808 and a lipoplex formation module 736 that are each fluidly coupled downstream of both a lipid 810 supply and a drug substance/RNA formation module 128, and are both fluidly coupled upstream of a buffer exchange and/or purification module 814. Stated otherwise, the module 10 may include a drug substance/RNA formation module 128 and a supply of lipids 810 that each supply (and are each upstream of) both an LNP formation module 808 and a lipoplex formation module 720. In the case of the lipoplex formation module 720, the supply of lipids 810 may be fluidly coupled upstream of the lipoplex formation module 720 with the liposome formation module 700 being disposed between the supply of lipids 810 and the lipoplex formation module 720. Liposomes 736 (formed in the liposome formation module 700 using the lipids 810) may then be routed from the liposome formation module 700 to the lipoplex formation module 720. Therefore, the supply of lipids 810 may be coupled directly to the liposome formation module 700.

Still referring to FIG. 26, the module 10 may include a buffer prep area 18 that received buffer ingredients that have entered the module via the material airlock 42. Buffer 812, which is prepared in the buffer prep area 18, may be used in the buffer exchange/purification module 814. Downstream of the buffer exchange/purification module 814, the module 10 may include a dilution/filtration module 816, which is described in further detail in connection with FIG. 13. As shown in FIG. 26, the module 10 may also include a shared use/shared equipment area 24 that may be used in connection with several of the module or workflow processes housed within the module 10. For example, the shared use/shared equipment area 24 may be operatively coupled to and/or accessible from each of the drug substance/RNA formation module 128, the liposome formation module 700, the lipoplex formation module 720 the buffer prep area 18, the buffer exchange/purification module 814, and/or the dilution/filtration module 816, as indicated by the dashed lines in FIG. 26 connecting the shared use/shared equipment area 24 to each of the aforementioned processes/process modules (or sub-modules). The shared use/shared equipment area 24 is described herein in further detail in connection with FIG. 24. The combined process flow 600 may also include other downstream processes that are located outside of the module 10 including (but not limited to) fill and finish 90 (described in connection with FIGS. 3, 13, and 25) transport, warehousing, and freezing 830 (described at least in connection with FIG. 13), and administration of the drug product to an end user 606. In some embodiments, as described herein, the drug product may be administered to the patient following fill and finish 90 (i.e., thereby avoiding the need to transport, warehouse, and/or freeze 830 the drug product). In some embodiments, for example, in embodiments involving small production runs (for example, for use in connection with individualized or personalized medicine, and/or smaller production runs of vaccines for treating/preventing rare diseases and/or localized strains of a disease), the drug product may be administered to the end user 606 immediately following dilution/filtration 816 (with no fill and finish 90, transport, warehousing, and/or freezing 830 steps occurring).

Referring still to FIG. 26, according to aspects of the present embodiments, the combined workflow/process flow 600 may also include lyophilization and/or autoclave steps that occur outside of the boundary of module 10 (for example, approximately contemporaneously with fill and finish 90 (i.e., in most cases, immediately before, and/or shortly before fill and finish 90), i.e., within or adjacent the fill and finish module 90 illustrated in FIG. 25). As illustrated in FIGS. 24 and 26, the buffer prep area 18 may be adjacent to and operatively coupled with both the LNP formation area 16, 808 and the lipoplex formation area 20, 700 while the shared area 24 may be adjacent to and operatively coupled with the LNP formation area 16, 808 the lipoplex formation area 20, 700 and the drug substance 22/RNA formation module 128.

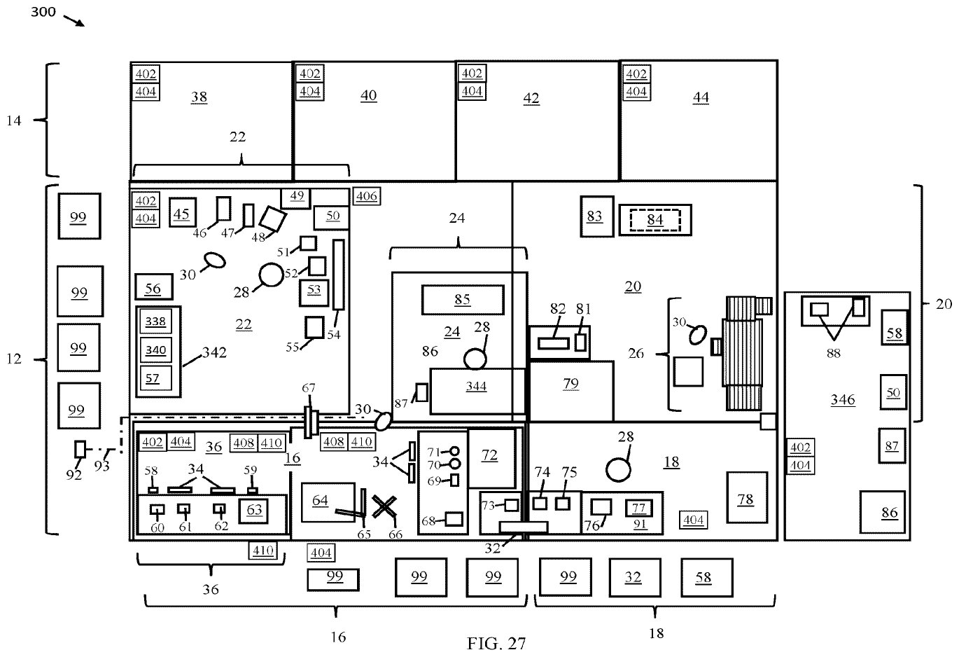

FIG. 27 depicts an overview of an exemplary drug product manufacturing site and/or module 300, according to aspects of the present disclosure. The exemplary drug product manufacturing site and/or module 300 illustrated in FIG. 27 is similar to the module 10 illustrated in FIG. 24, with some differences. In some embodiments, the exemplary drug product manufacturing site and/or module 300 includes a preparation area 346 used to house one or more scales, 88, a peristaltic pump 58, a cart 50 (for transporting materials and/or supplies), an HMI (to allow users to access a computer/network/database, etc., and/or mixing equipment 86 (for example, an Allegro mixing unit). In some embodiments, the preparation area 346 (or dispensing room 346) may further include one or more pressure sensors 402 and/or one or more combined temperature and humidity sensors 404. In some embodiments, dispensing room 346 (or preparation area 346) may further include one or more roller shutter doors for transferring materials between the dispensing room 346 and the interior of the drug product manufacturing site and/or module 300 (and/or fill and finish module 360, as the case may be). In some embodiments, the exemplary drug product manufacturing site and/or module 300 includes one or more peristaltic pumps 58 and one or more filter holders 32 around the periphery of the module 300. In some embodiments, the exemplary drug product manufacturing site and/or module 300 includes a bench 342 in the drug substance area 22 for supporting a mixer, shaker and/or bioreactor (for example, a Varioshake shaker) 338, a centrifuge 340, and/or the chromatography unit 57.

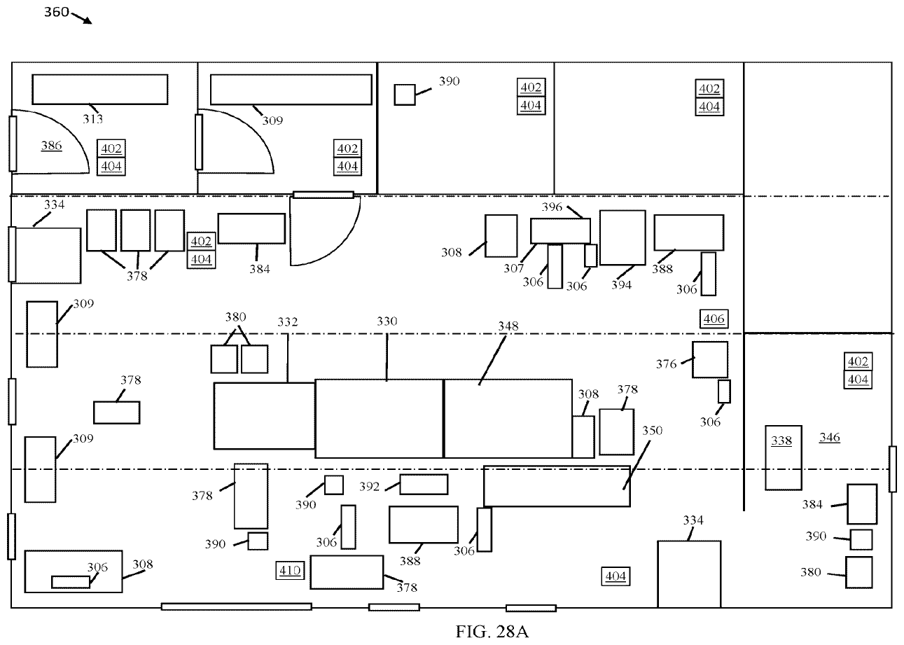

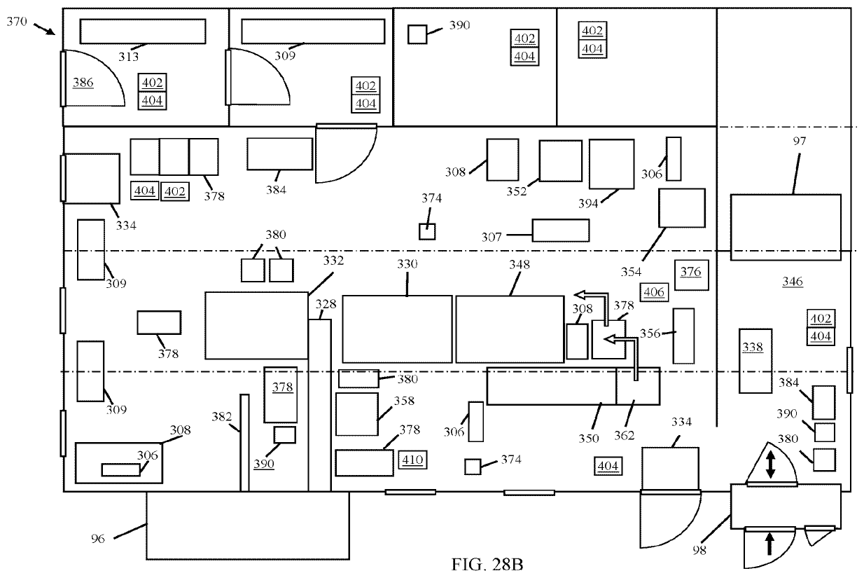

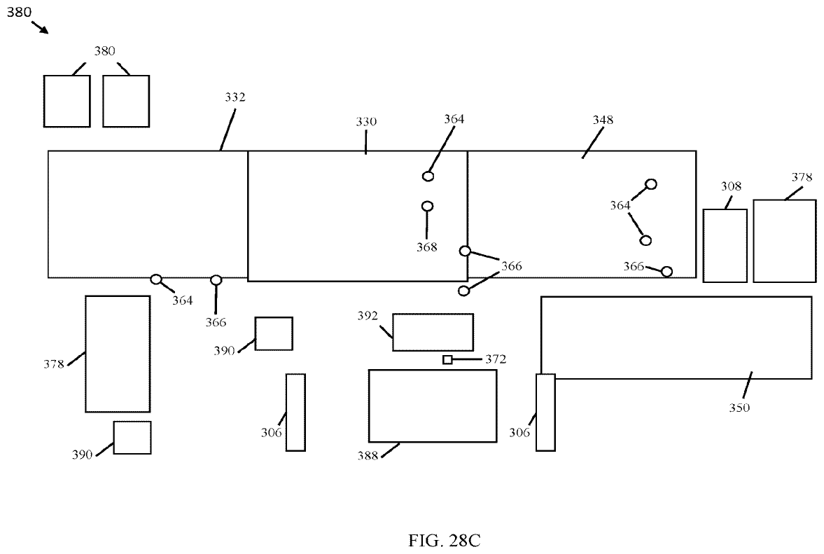

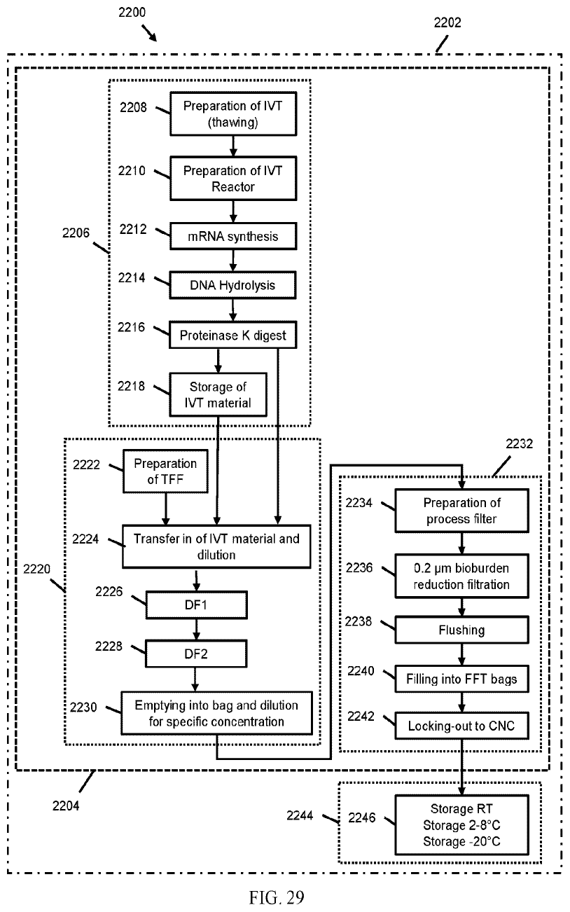

FIG. 28A depicts an overview of an exemplary fill and finish module 360, according to aspects of the present disclosure. The exemplary fill and finish module 360 depicted in FIG. 28A is similar to that of exemplary fill and finish module 90 depicted in FIG. 25, with configuration differences, as described herein. In some embodiments, the exemplary fill and finish module 360 may include debagging machinery 350, a denesting machine 348, a filling machine 330, a capping and crimping machine 332, utility services 306, refrigerator and/or bag testers 307, a desk 308, one or more cabinets 309, a preparation area (“prep area”) 346 and/or dispensing area 346, and/or a glove box tester 338 (in the dispensing area 346), among other equipment and machinery as described herein. In some embodiments, the exemplary fill and finish module 360 includes multiple trolleys or carts 378, 380 for easily transporting various work products and/or materials such as containers (such as carboys) containing hydrogen peroxide used for one or more decontamination processes. In some embodiments, the trolleys or carts 378, 380, may be used for carrying flexible bags, caps, container tops, vials, containers, and/or other supplies used in connection with fill and finish processes, as described herein. In some embodiments, the exemplary fill and finish module 360 includes multiple mobile waste bins or receptacles 390 dispersed throughout the module 360. In some embodiments, the exemplary fill and finish module 360 includes one or more small carts 392 housing a peristaltic pump and/or a filtration rack 394 (for example, a mobile filtration rack 394) used for mounting a single-use filtration set thereto. In some embodiments, the exemplary fill and finish module 360 includes a large cart 396 housing a biowelder and/or a biosealer such that the various components may be selectively fluidly connected and/or disconnected to each other, as needed by the particular production run being performed and/or as a result of process parameters being monitored.

FIG. 28B depicts an overview of an exemplary fill and finish module 370, according to aspects of the present disclosure. The exemplary fill and finish module 370 depicted in FIG. 28B is similar to that of exemplary fill and finish module 360 depicted in FIG. 28A, with configuration differences, as described herein. Whereas the fill and finish module 360 depicted in FIG. 28A may represent more of a normal use case, the fill and finish module 370 depicted in FIG. 28B represents more of a “maximum use” case. In some embodiments, the exemplary fill and finish module 370 may include debagging machinery 350, a denesting machine 348, a filling machine 330, a capping and crimping machine 332, utility services 306, refrigerator and/or bag testers 307, a desk 308, one or more cabinets 309, a prep area 346 and/or dispensing area 346, and/or a glove box tester 338 (in the dispensing area 346), among other equipment and machinery as described herein. In some embodiments, the denesting machine 348 may also be used for delidding. In some embodiments, the debagging machine 350 may also be used for tub-feeding. In some embodiments, the glove box tester 338 may be used for glass integrity testing in the dispensing area 346).

Referring still to FIG. 28B, in connection with the maximum use case depicted in FIG. 28B, the exemplary fill and finish module 370 may also include an autoclave 97, lyophilization equipment 96, dish, glass, ceramic, polymer and/or metallic (i.e., stainless steel) component washing equipment 98, a temperature control unit (TCU) 358, and other equipment 356 (i.e., a drive unit for the magnetic stirrers within the mixing units 352, 354). In some embodiments, for example, in connection with processes that include use of the lyophilization equipment 96, the vials are first semi-stoppered (i.e., rather than fully stoppered). The exemplary fill and finish module 370 may also include a first mixing unit 352 (for example, a ProMixer mixing unit) as well as a second mixing unit 354. In some embodiments, in addition to, or instead of the first mixing unit 352, the exemplary fill and finish module 370 may include one or more bioprocessing vessels that may be cooled or heated, in cases where a single bulk drug product (bDP) batch (for example, 8 L to 10 L batch) needs to be cooled and/or heated, but not mixed. The first and/or second mixing units 352, 354 may be used to pool and subsequently mix up to 5 different types of RNA drug product for use in a single vaccine (for example, for use in a cancer vaccine), i.e., prior to fill and finishing steps. For example, in some embodiments, the first and/or second mixing unit 352, 354 may be used to pool, mix, and/or combine six (6) bags, each with a volume of about eight (8) liters, each containing different RNA (i.e., different mRNA), for us in a single multivalent product. In some embodiments, the bags with different volumes may be used (for example, from about 1 L to about 50 L and other volumes therebetween such as 2 L, 5 L, 10 L, 12 L, 15 L, 20 L, 25 L, 30 L, 40 L, etc.). In some embodiments, the first and/or second mixing unit 352, 354 may be used to pool multiple batches of the same mRNA (for example to pool different batches together to create a larger volume for filling). In some embodiments, the first mixing unit 352 is used for pooling and the second mixing unit 354 (downstream of the first mixing unit 352) is used for sterile filtration of the bulk drug product. In some embodiments, the first and/or second mixing unit 352, 354 may include an integrated (or separate) cooling unit and/or a scale.

Still referring to FIG. 28B, the exemplary fill and finish module 370 may further include an air bathing area 362 (i.e., a Zone 1 area 362) for bathing the bulk drug product bags with Grade A air prior to and during the debagging process (i.e., at or prior to the debagging machine 350) to reduce the likelihood of contamination of the bulk drug product. Conduits, ducts, louvers, and/or vents from the ceiling of the fill and finish module 370 may be used to direct Grade A air over the air bathing area 362 (i.e., the Zone 1 area 362) such that each bulk drug product bag is entirely bathed in Grade A air during the debagging process. In some embodiments, the Grade A air provided in the air bathing 362 (i.e., the Zone 1 area 362) is provided as laminar flow such that the Grade A air does not cause any disturbance to the bulk drug product as it is debagged. In some embodiments, the Grade A air is provided in the air bathing area 362 (i.e., the Zone 1 area 362) to further de-risk processes that are more susceptible to contamination. In some embodiments, Grade A air is circulated throughout the entire fill and finish module 370 (or drug substance/drug product module 300, as the case may be) and an additional volume of Grade A air is directed to the Zone 1/air bathing area 362 as laminar flow for processes that require double bagging (and double debagging). Debagging/removal of the outer bag occurs within the Zone 1/air bathing area 362 while debagging of the inner bag may occur within the debagging machine 350.

Referring still to FIG. 28B, in some embodiments, the exemplary fill and finish module 370 is sanitized manually. In some embodiments, the exemplary fill and finish module 370 includes a sanitizing system (for example, an automated sanitizing system) 374. In some embodiments, the sanitizing system 374 is positioned in one or more locations within the exemplary fill and finish module 370 such that each piece of machinery and/or equipment may be reached by the sanitizing system 374. In some embodiments, the sanitizing system 374 is suspended from the ceiling such that it does not hinder movement of people and materials through and around the exemplary fill and finish module 370. In some embodiments, the sanitizing system 374 includes a misting sanitization system capable of disbursing a sanitizing mist throughout the exemplary fill and finish module 370. In some embodiments, the sanitizing system 374 includes one or more SteraMist units. In some embodiments, the sanitizing system 374 vaporizes hydrogen peroxide and provides a sterilizing mist throughout the exemplary fill and finish module 370. In some embodiments, the sanitizing system 374 provides a sanitizing mist that is able to get inside the machines of the exemplary fill and finish module 370. In some embodiments, the sanitizing system 374 is a sterilization in place (SIP) system. In some embodiments, the sanitizing system 374 provides a sterilizing mist that includes ionized hydrogen peroxide. In some embodiments, the sanitizing system 374 described herein in connection with the fill and finish modules 90, 360, 370, 380 may also be used in connection with the drug substance/drug product module(s) 10, 300. The exemplary fill and finish module 370, as well as drug substance/drug product module(s) 10, 300 as described herein may also be cleaned and/or sanitized manually. In some embodiments, the exemplary fill and finish modules 90, 360, 370 may be used to fill 10,000 vials per batch with 1 mL per vial of drug product. In some embodiments, the exemplary fill and finish modules 90, 360, 370 may be used to fill up to 50,000 vials per batch with 1 mL per vial of drug product. In some embodiments, the exemplary fill and finish modules 90, 360, 370 depicted in FIGS. 25, 28A, and 28B, as well as the drug substance/drug product modules 10, 300 depicted in FIGS. 24 and 27 include a total Clean Room grade C area (i.e., floor area) of about 100 square meters (for example, from about 80 square meters to about 120 square meters). In some embodiments, the exemplary fill and finish modules 90, 360, 370 depicted in FIGS. 25, 28A, and 28B include a drive unit 356 including one or more motors for actuating the magnetic stirrer of the first and/or second mixing units 352, 354 (shown in FIG. 28B). In some embodiments, the drive unit 356 fits between the Zone 1 area 362 (i.e., air bathing area 362) and the wall of the fill and finish module 90, 360, 370.

Referring to FIGS. 28A and 28B, the exemplary fill and finish module 360, 370 may include multiple sensors including pressure sensors 402, individual and/or combined temperature and humidity sensors 404, hydrogen peroxide (H₂O₂) sensors 406, and oxygen (O₂) sensors 410 positioned at various locations, as shown in FIGS. 28A and 28B. The fill and finish module 360, 370 shown in FIGS. 28A and 28B may also include one or more entryways 386, a cabinet area with a sink 313 (for example, within the personnel airlock area 38), one or more marked areas 334, one or more large carts 378, one or more small carts 380, a large cart with a mobile bioreactor coupled thereto 388, one or more mobile HMI docking stations 384, a conveyor system 382 (shown in FIG. 28B, for example, for transporting vials/fluid vessels to the lyophilization equipment 96), and/or a fixed HMI 376.

FIG. 28C depicts an enlarged overview of an exemplary fill and finish module 380, according to aspects of the present disclosure. The exemplary fill and finish module 380 of FIG. 28 C depicts various utility services that are provided in connection with the fill and finish process 380. For example, the exemplary fill and finish module 380 may include two compressed air supplies or conduits 364 providing compressed air to the denesting machine 348, as well as a single compressed air supply 364 providing compressed air to each of the filling machine 330 and the capping and crimping machine 332. In some embodiments, the exemplary fill and finish module 380 may include at least one exhaust air conduit 366 positioned adjacent each of the denesting machine 348, the filling machine 330, and the capping and crimping machine 332. In some embodiments, the exemplary fill and finish module 380 may include at least two exhaust air conduits positioned between (or at the interface between) the denesting machine 348 and the filling machine 330. In some embodiments, the exemplary fill and finish module 380 may include a nitrogen (N₂) supply 368 (i.e., for use as purge gas and/or for establishing and/or maintaining inert conditions) disposed at or above the filling machine 330 thereby supplying nitrogen thereto. In some embodiments, the nitrogen supply comprises substantially pure nitrogen (N₂). In some embodiments, the nitrogen supply includes at least 99% nitrogen (N₂) on a molar basis. In some embodiments, the exemplary fill and finish module 380 may also include one or more Harting connectors 372 and/or other utility services 306 as described herein. The large cart with a mobile bioreactor coupled thereto 388, along with multiple large carts 378 and multiple small carts 380 are also shown in FIG. 28C. Each of the modules depicted in FIGS. 24, 25, 27, and 28A-28C may include a second layer or level of containers for use as HVAC and/or as a technical area (that is, similar to the 2-level embodiments shown in FIG. 4). For example, in some embodiments, one or more containers on the second level may be used for HVAC while one or more containers on the second level may be used as a technical area. In some embodiments, the second level may include a technical area used for post-use integrity testing (i.e., to assess the integrity of the filters (for example, AquaWIT filters from Palltronic)) used in connection with the processes described herein. In some embodiments, the second level may be used for storage of equipment that is not being used for a particular production run. In some embodiments, each of the utilities as described herein is provided with redundancy.

The present disclosed embodiments provide several potential advantages. The modular and/or portable drug production systems, modules, and facilities of the present embodiments allow a variety of drugs to be produced, and enable flexibility in the delivery modality (for example, RNA-based products delivered via LNP, liposome, and/or lipoplex delivery systems). The modular and/or portable drug production systems, modules, and facilities of the present embodiments provide flexibility on being able to be used for drug production, as well as in research and development environments. Because of the layout of drug product manufacturing site and/or module 10 (shown in FIG. 24), drug substance may easily be delivered from the drug substance module 22 to either the LNP formation module 16 or the lipoplex formation module 20 (i.e., the drug substance module 22 is adjacent to, and accessible from, each of the LNP formation module 16 and the lipoplex formation module 20, and vice versa). Similarly, the buffer prep area 18 is also located adjacent to each of the LNP formation module 16 and the lipoplex formation module 20, thereby facilitating the ease with which buffer can be provided to each of the drug production workflows (i.e., LNP module 16 and lipoplex module 20). The shared use area 24 is adjacent to, and accessible from, each of the LNP formation module 16, the lipoplex formation module 20, and the drug substance module 22, and therefore both serves those modules but also enables adaptive workflows such that processes or tasks may seamlessly be moved to and from the shared area 22 as needed to streamline operations of the drug product manufacturing site and/or module 10, which may experience frequent changes in operations and/or workflows. In addition, having a fill and finish module 90 located at the same production facility as the drug substance and drug production modules 22, 16, 20 reduces and/or eliminates the need for intermediate freezing, storages, and/or transport steps between drug formation and fill and finish 90. The airlock area 14 of the modular and/or portable drug production systems, modules, and facilities of the present embodiments also enables both personnel and materials to move or be moved in and out of the modules while maintaining clean room (for example, Grade C clean room) conditions.

As disclosed herein, in some embodiments, the fill and finish module 360 may include an automated filling machine 330. In some embodiments, the automated filling machine 330 may include a VarioSys automated filling machine (for example, a model KSF 5105 machine from VarioSys or a model TDF 9105 machine from VarioSys). In some embodiments, the automated filling machine 330 may include other makes and models of filling machines.

Each of the drug substance/drug product modules 10, 300 and the fill and finish module 90, 360, 370, 380 includes various utilities including electricity (for example, 0-10V service, 110V-240V service, 110V-400V service, 110V-440V service, and/or 4-20 mA service), process air (for example, technical compressed air (AII) and compressed air (AIP) for process use (i.e., product transfer), internet/ethernet connections (including wireless connections, ProfiNet, LAN, WAN, WLAN, and other types of connections), water for injection (WFI), steam (i.e., clean steam for sterilization), wastewater conduits, condensate return lines, nitrogen (N₂) for purge, and/or Harting connections (i.e., providing serial port connections to accommodate multi-system plugs). In some embodiments, the Profibus standard is used in connection with equipment within the modular drug substance/drug product module and the fill and finish as part of a process control system. Each of the utility services including air ducts (air inlets and outlets) and other types of utilities, where applicable, may be routed through the walls, floors, and/or ceilings.

FIG. 29 depicts an overview of an exemplary drug substance workflow (or method) 2200, according to aspects of the present disclosure. The method 2200 may generally include in vitro transcription 2206, tangential flow filtration (TFF) 2220 and bioburden reduction 2232. At step 2208, the method 2200 may include preparation of IVT materials (for example, thawing of stock DNA input material(s)). At step 2210, the method 2200 may include preparation of a bioreactor for in vitro transcription (IVT). At step 2212, the method 2200 may include performing an mRNA synthesis process (for example, using the stock DNA to form mRNA via syntheses within the bioreactor). At step 2214, the method 2200 may include performing a DNA hydrolysis process (for example, to initiate a DNase reaction and to halt the in vitro transcription process). At step 2216, the method 2200 may include digestion of proteinase K (for example, in order to reduce the size of proteins in the reaction mixture). At step 2218, the method 2200 may optionally include storage of the IVT solution. For example, in some embodiments, IVT solution is placed in storage following digestion of proteinase K at step 2216. In some embodiments, IVT solution is delivered directly to the TFF module 2220 following digestion of proteinase K at step 2216. In some embodiments, a chromatography step may also be performed following digestion of the proteinase K (at step 2216) and prior to delivery of the IVT solution to the TFF module 2220.

Referring still to FIG. 29, at step 2222, in connection with tangential flow filtration (TFF) 2220, the method 2200 may include preparation of the TFF module. At step 2224, the method 2200 may include transfer of the IVT material (i.e., IVT solution from the IVT process 2206) into the TFF module 2220 and dilution of the IVT material. At step 2226, the method 2200 may include a first diafiltration process. At step 2228, the method 2200 may include a second dialfiltration process. At step 2230, the method 2200 may include disposing the output of the second dialfitration process into a container (for example, a flexible bag) and diluting the output to a specific concentration. At step 2234, in connection with a bioburden reduction module or process 2232, the method 2200 may include preparation of a process filter. At step 2236, the method 2200 may include performing bioburden reduction filtration (for example, using a 0.2 micron filter). At step 2238, the method 2200 may include a flushing process. At step 2240, the method 2200 may include filling bags with the filtered solution from step 2238. At step 2208, the method 2200 may include locking-out to CNC (i.e., the BioNTainer hall). Locking-out to CNC includes moving the drug substance (i.e., the output of the bioburden reduction process/module 2232) from within a clean room class C (that is “CRC”) area 2204 to outside of the CRC area 2204 to a controlled not-classified (that is, “CNC”) area 2244. In some embodiments, each step of method 2200 (including storage 2246) is performed within a shell building 2202 or module boundary while a subset of the steps are performed within the CRC area 2204.

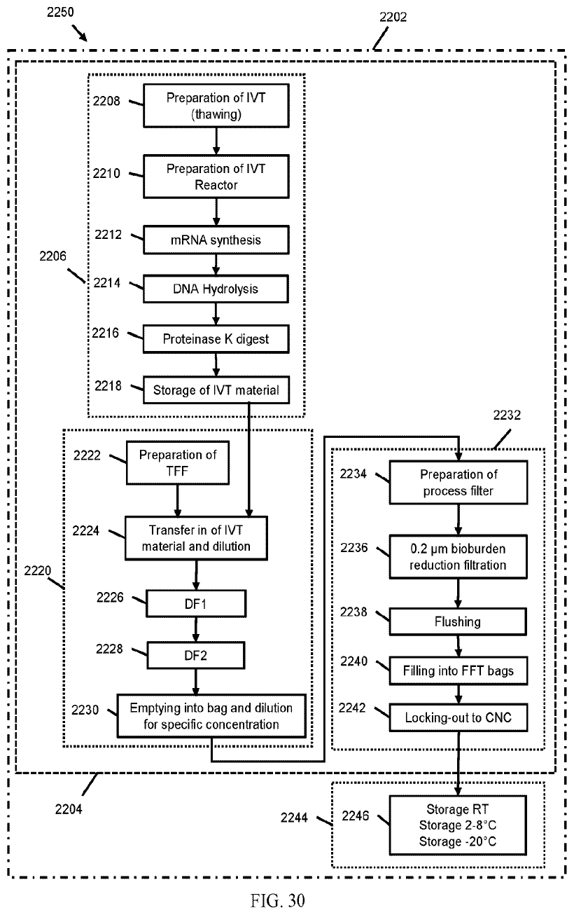

FIG. 30 depicts an overview of an exemplary drug substance workflow 2250, according to aspects of the present disclosure. The embodiment of the dug substance workflow 2250 exemplified in FIG. 30 differs from FIG. 29 only at steps 2216 and 2224 in that in the embodiment of FIG. 30 (which may be more commonly used in connection with cancer vaccines vs. infectious diseases for FIG. 29) the IVT solution is stored after digestion of proteinase K at step 2216 whereas in FIG. 29 the IVT solution may be delivered directly to the TFF module 2220 for immediate use in one or more diafilitration processes 2226, 2228.

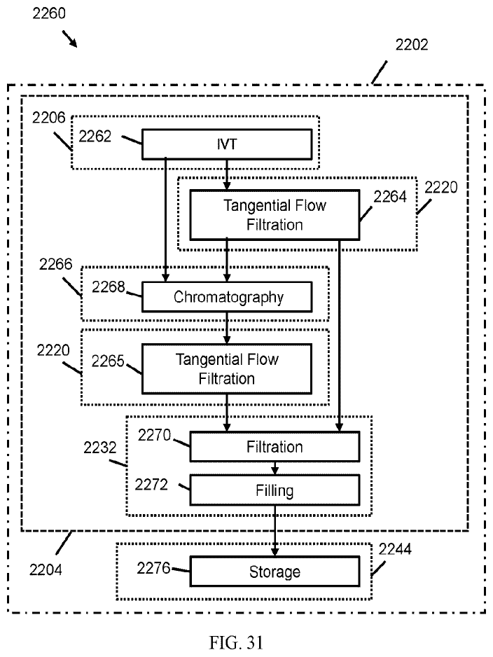

FIG. 31 depicts an overview of an exemplary drug substance workflow 2260, according to aspects of the present disclosure. The drug substance workflow 2260 depicted in FIG. 31 includes the IVT module/workflow 2206, the TFF workflow/module 2220, and the bioburden reduction (BBR) workflow/module 2232 of FIGS. 29 and 30 with additional steps (for example, chromatography 2268) to allow for and/or accommodate various processes (i.e., drug substances to be used for cancer vaccines) that require additional purification. Accordingly, within a single module 2202, multiple workflows are possible. For example, in a first DS workflow, the process is initiated with in vitro transcription (IVT) 2262 and proceeds to chromotagrapy at step 2268, followed by tangential flow filtration at step 2264, followed by filtration 2270, filling 2272 and storage 2276. In a second DS workflow, the process is initiated with in vitro transcription (IVT) 2262 and proceeds to a first TFF process at step 2264, followed by chromotagrapy at step 2268, followed by a second tangential flow filtration (TFF) process at step 2265, followed by filtration 2270, filling 2272 and storage 2276. In a third DS workflow, the process is initiated with in vitro transcription (IVT) 2262 and proceeds to a TFF process at step 2264 and then directly to filtration 2270, filling 2272 and storage 2276 (that is, skipping chromatography 2268 and the second TFF 2265). In summary, some processes include chromatography 2268 and some do not. In addition, of the processes that include chromatography 2268, some may include a TFF process 2264 that is performed before chromatography 2268 in addition to the TFF process 2265 that is performed after chromatography 2268. As shown in FIG. 31, each of the steps of the workflow 2260 is performed within the CRC area 2204 (i.e., clean room grade C) except for storage 2276, which may occur outside of the CRC area 2204 but within the BioNTainer (i.e., within the CNC area 2202 (that is, within the Controlled Not Classified (CNC) area (i.e., the BioNTainer hall))).

Referring still to FIG. 31, according to aspects of the present disclosure, in some embodiments, the method 2260 may include performing filtration 2270 directly after the first TFF step 2220. In some embodiments, the method 2260 may include performing chromatography 2268 directly after the first TFF step 2264, and then performing a second TFF step 2265 after the chromatography step 2268. In some embodiments, the method 2260 may include performing chromatography 2268 directly after the first IVT step 2262 (i.e., not performing TFF 2264 after the IVT step 2262) and performing only a single TFF step 2265 following chromatography 2268.

Still referring to FIG. 31, in some embodiments in which chromatography 2268 is performed, dsRNA (double-stranded RNA), dsDNA (double-stranded DNA), oligo (dT) primers, etc. may be obtained (i.e., removed from the drug substance) as a side product during (or after) in vitro transcription, as described herein. In some embodiments, the process and/or drug substance/drug product being produced may require additional purification steps (for example, bead-based purification (i.e., magnetic bead-based chromatography), etc.). In some embodiments, chromatography may be used to remove residual primers (for example, oligo (dT) primers, i.e., oligonucleotides that include repeating deoxythymidines segments, i.e., used for annealing with the poly A tail of one or more mRNA strings (for example, for use with DNA synthesis from complementary RNA)). Therefore, in some such embodiments, and as described herein, chromatography 2268 may be used in addition to, or instead of, one or more tangential flow filtration 2264 processes, i.e., to remove dsRNA, dsDNA, oligo (dT) primers, and other contaminants. In some embodiments, chromatography 2268 may also be used to enrich mRNA with a long dT tail. For example, in some embodiments, cellulose materials (e.g., microcrystalline cellulose) may be used to remove dsRNA contamination, for example, in some embodiments in a chromatographic format (i.e., in connection with chromatography step 2268). In some embodiments, cellulose materials (e.g., microcrystalline cellulose) can be pretreated to inactivate potential RNase contamination, for example in some embodiments by autoclaving followed by incubation with aqueous basic solution, e.g., NaOH. In some embodiments, cellulose materials may be used to purify RNA molecules according to methods described in WO 2017/182524, the entire content of which is incorporated herein by reference. In some embodiments, testing bulk drug product (bDP) for dsRNA, dsDNA, oligo (dT) primers, etc. occurs in the technical area located on the second level (i.e., above the drug substance/drug product module 300).

Referring still to FIG. 31, in some embodiments, the method/workflow may make use of a biowelder 82 and/or a biosealer 81 (both shown in FIG. 24). Biowelder 82 and/or a biosealer 81 equipment may be used in connection with the drug substance workflows (for example, workflow 2260 shown in FIG. 31) described herein when additional filtration or purification is determined to be required while a workflow is being performed. For example, biowelder 82 and/or a biosealer 81 equipment may be used to selectively route flow (i.e., flow of a drug substance solution) “on the fly” (for example, in real time or near real time) from an IVT module 2262 or a TFF module 2264 to a chromatography module 2268 and/or a TFF module 2264, 2265 in an aseptic manner that minimizes that chances of contamination. For example, if a batch of drug substance fails to meet one or more purification metrics (i.e., acceptance criteria) the batch may be asseptically routed to a chromatography unit using biowelder 82 and/or a biosealer 81 equipment. The biowelder 82 may include one or more single use blades and multiple tube holders within an enclosure to cut a first tube (for example, from the upstream process (i.e., a TFF or IVT unit)) and a second tube (for example, from a downstream process (i.e., TFF and/or chromatography)) and then to establish a connection between the first tube and the second tube within the enclosure, all while maintaining aseptic conditions, (i.e., such that from a bioprocessing perspective, the system is maintained as a “closed system” even while flows are being rerouted). In some embodiments, a biosealer may also be used to ensure aseptic conditions are maintained while flows are being selectively rerouted. In some embodiments, the biowelder is fully automated and accomodates tube outer diameters in a range from about 0.25 inches to about 1 inch. By using biowelder 82 and/or a biosealer 81 equipment, sterile connections can be made while maintaining a “closed system,” even when performed on tubing that contains drug substance and/or other biological solutions.

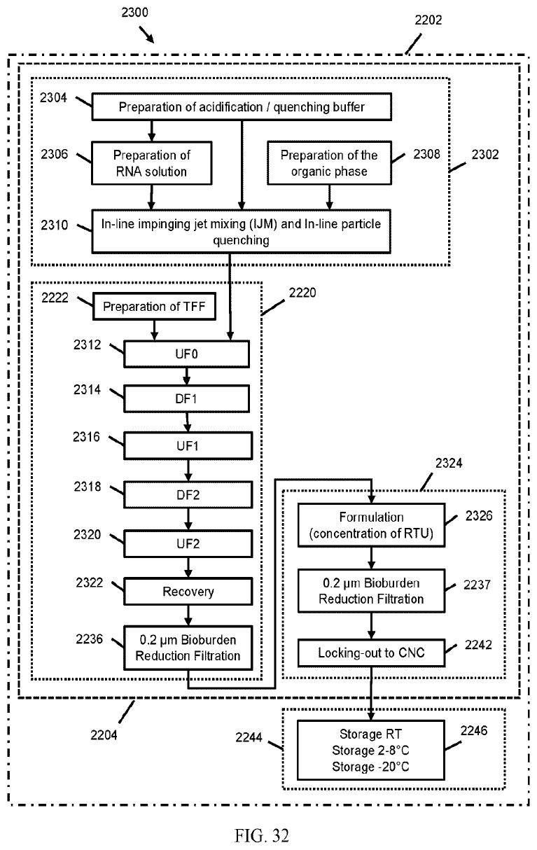

FIG. 32 depicts an overview of an exemplary drug product workflow 2300, according to aspects of the present disclosure. The drug product workflow 2300 (or method 2300) may generally include impingement jet mixing 2310 performed within an LNP formation module 2302, tangential flow filtration (TFF) performed within a TFF module 2220, bioburden reduction (BBR) 2236 performed within a BBR module 2324, and storage 2246 in a storage area 2244. At step 2304, the method 2300 may include preparation of an acidification and/or quenching buffer, which may be used (optionally) at step 2306 (preparation of an RNA solution) and/or directly at step 2310 (in-line impingement jet mixing and in-line particle quenching). Preparation of the RNA solution at step 2306 employs the RNA (i.e., in some embodiments, being in the form of an RNA solution (i.e., drug substance)) that was formed via the drug substance workflow 2200 described in connection with FIG. 29. At step 2308, the method 2300 may include preparation of the organic phase, which is also used at the LNP step 2310 (i.e., during impingement jet mixing 2310). Once lipid nanoparticles (LNP) (i.e., RNA-LNPs, i.e., mRNA-LNPs) are formed within the LNP formation module 2302, the RNA-LNP solution is delivered to the TFF module 2220 for tangential flow filtration. At step 2222, the method 2300 may include preparation of the TFF equipment. At step 2312, the method 2300 may include a first ultrafiltration step. At step 2314, the method 2300 may include a first diafiltration step. At step 2316, the method 2300 may include a second ultrafiltration step. At step 2318, the method 2300 may include a second diafiltration step. At step 2320, the method 2300 may include a third ultrafiltration step. At step 2322, the method 2300 may include a recovery step (for example, including a buffer (i.e., a Tris buffer) in some embodiments) to extract tangential flow filtrated drug product from the TFF equipment/cassette(s). In some embodiments, recovery 2322 may include use of a diaphragm pump and/or a 50 L flexible bag. At step 2236, the method 2300 may include 0.2 micron bioburden reduction filtration (i.e., while the drug product solution is still within the TFF module 2220). Following tangential flow filtration, the drug product solution is delivered to the bioburden reduction module 2324 where a formulation step 2326 is performed (to arrive at a ready to use (RTU) concentration of the drug product). At step 2237, the method 2300 may include performing another 0.2 micron bioburden reduction filtration process after which the drug product is removed from the CRC (clean room grade C) area 2204 and is stored in a storage area 2244 (step 2246), which is outside the CRC area 2204, but within the CNC (“controlled not classified”) area 2202. At step 2246, storage of the drug product may include storage at room temperature, storage in a range from about 2° C. to about 8° C., and/or storage at about −20° C.

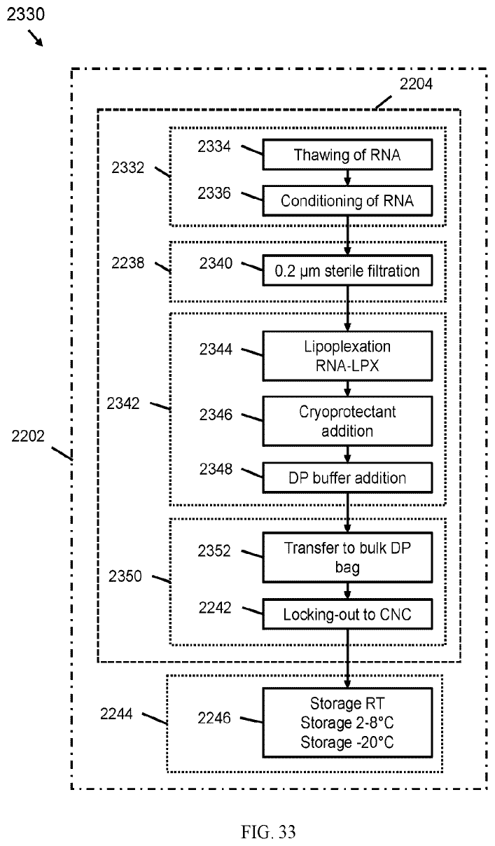

FIG. 33 depicts an overview of an exemplary drug product workflow 2330 (or method 2330), according to aspects of the present disclosure. In contrast to the drug product workflow 2300 depicted in FIG. 32 (which may be used to form RNA-LNP drug products, for example, for use in infectious disease vaccines), the drug product workflow 2330 depicted in FIG. 33 may be used to form lipoplexes (i.e., RNA lipoplexes, for example, for use with cancer vaccines, among other types of drug products that employ lipoplexes as the delivery modality). The method 2330 may generally include preparing the RNA 2332, filtration 2238, lipoplex formation 2342, transport of the drug product 2350, and storage 2244. At step 2332, the method 2330 may include preparing the RNA (i.e., the RNA, in some embodiments, being in the form of an RNA solution/drug substance (for example, formed via the drug substance workflow 2250 described in connection with FIG. 30)). In some embodiments, preparing the RNA includes thawing the RNA 2334 and/or conditioning the RNA 2336, as described herein (for example, in connection with step 724 and FIG. 23). At step 2340, the method 2330 may include performing 0.2 micron sterile filtration on the conditioned RNA from step 2336. At step 2344, the method 2330 may include lipoplexation (i.e., to form lipoplexes as described herein, for example, in connection with step 726 and FIG. 23). At step 2346, the method 2330 may include the addition of cryoprotectant. At step 2348, the method 2330 may include the addition of buffer (for example, drug product buffer). At step 2352, the method 2330 may include transferring the bulk drug product/solution to one or more bags (for example, a 12 L bag, a 25 L bag, a 50 L bag, and/or other suitably sized bags). At step 2242, the method 2330 may include locking the drug substance out of the CRC (clean room grade C) area 2204 to the CNC (controlled not classified) area 2202 (i.e., the BioNTainer hall 2202). At step 2246, storage of the drug product may include storage at room temperature, storage in a range from about 2° C. to about 8° C., and/or storage at −20° C.

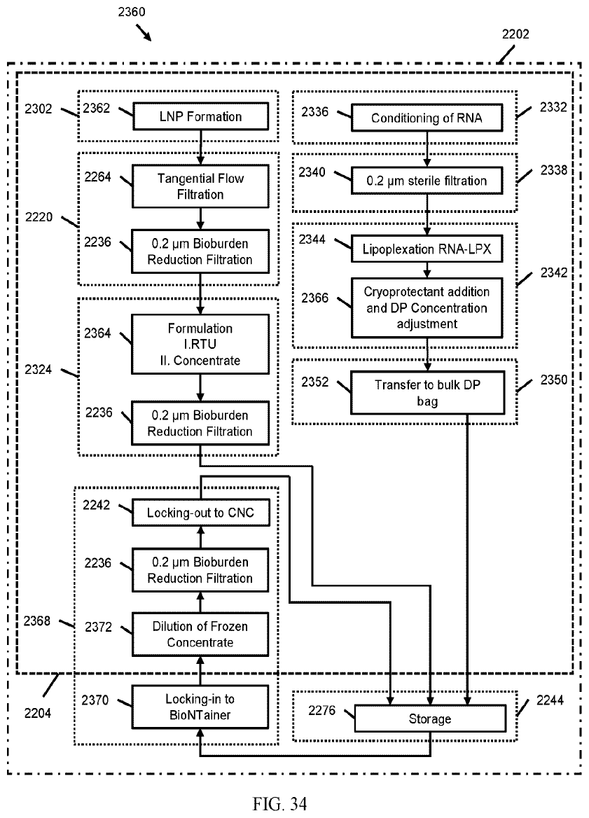

FIG. 34 depicts an overview of an exemplary drug product workflow 2360, according to aspects of the present disclosure. The drug product workflow 2360 (or method 2360) shown in FIG. 34 includes both the lipid nanoparticle (LNP) formation method 2300 illustrated in FIG. 32 as well as the lipoplex formation method 2330 illustrated in FIG. 33. FIG. 34 also illustrates various paths materials can take into and out of the clean room grade C area 2204, for example to and from the storage area 2244. Accordingly, according to aspects of the present disclosure, the combined drug substance and drug product workflows depicted in FIGS. 31 and 34 (which themselves collectively cover each of the workflows shown in FIGS. 29, 30, 32, and 33) are configured to fit into (i.e., to be disposed within) the modular drug production systems/units depicted in FIGS. 24 and/or 27.

Referring still to FIG. 34, the method 2360 may include LNP formation 2362, TFF 2264, bioburden reduction 2236, formulation and concentration 2364, and another bioburden reduction filtration step 2236 as part of the LNP formation workflow. The method 2360 may also include conditioning of RNA 2336, 0.2 micron sterile filtration 2340, lipoplexation 2344, cryoprotectant addition and drug product (i.e., bulk drug product (bDP)) concentration 2366, and transferring of drug product (i.e., bulk drug product) to bags 2352, as part of the lipoplex formation workflow. At step 2276, the method 2360 may include sending each of the LNP and/or lipoplex drug products to the storage area 2244. The storage area 2244 may be located outside the clean room grade C (CRC) area 2204 but still within the container/building/drug production area (i.e., CNC area) 2202 (i.e., the BioNTainer hall 2202). Any materials that are brought into the drug production area (i.e., CRC area) 2204 from storage 2276 at step 2370 may be locked into the BioNTainer (i.e., into the drug production area) via the material air lock (MAL) 42, 44 shown in FIG. 24 such that clean room/aseptic conditions may be maintained. At step 2372, the method 2360 may include dilution of frozen concentrate. At step 2236, the method may include 0.2 micron bioburden reduction filtration followed by locking out to the CNC area (step 2242).

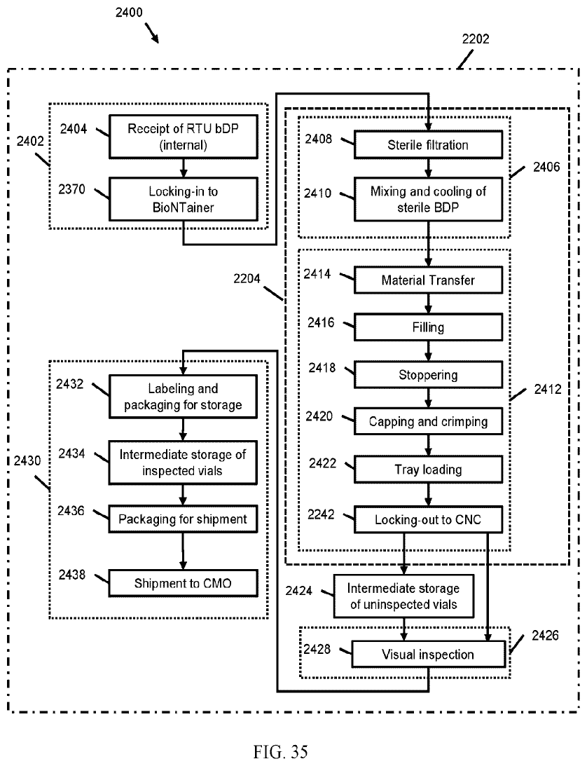

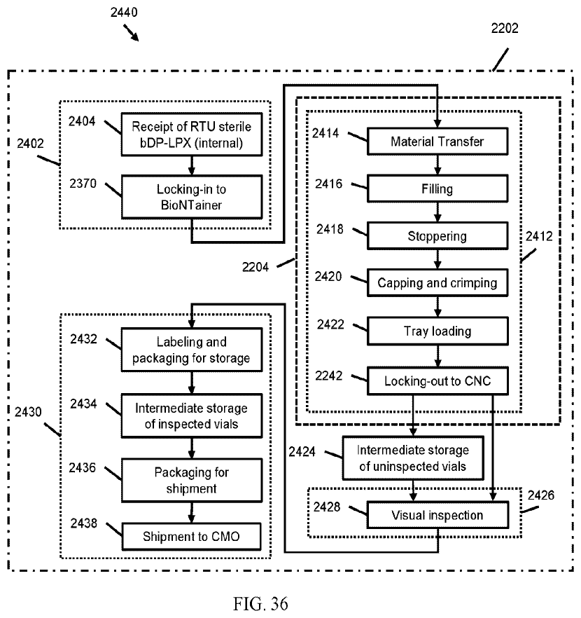

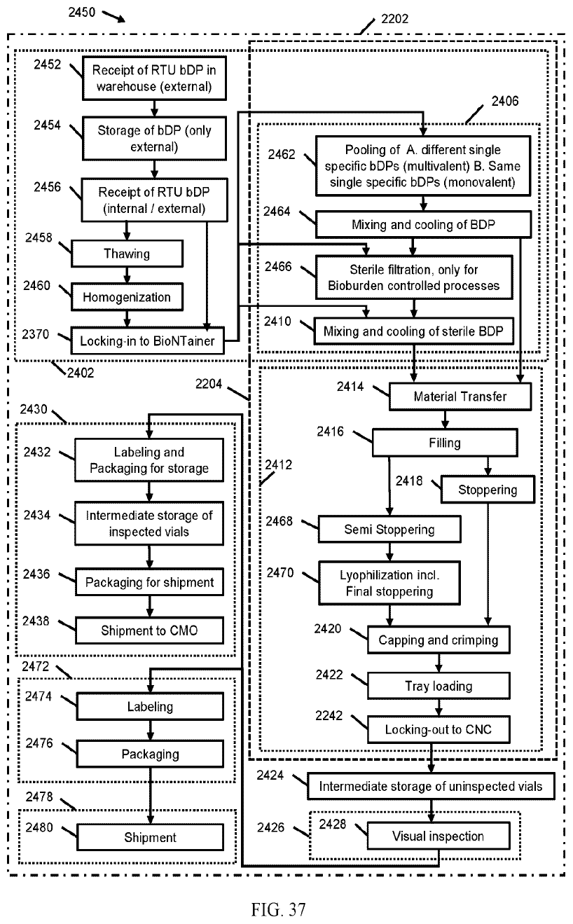

FIGS. 35-37 depict an overview of exemplary fill and finish workflows 2400, 2440, 2450, according to aspects of the present disclosure. The fill and finish workflow 2400 (or method 2400) of FIG. 35 primarily focuses on methods used in connection with infectious disease vaccines and/or for drug products that include a lipid nanoparticle (LNP) delivery modality. The fill and finish workflow 2440 of FIG. 36 primarily focuses on methods used in connection with cancer vaccines and/or for drug products that include a lipoplex (LPX) delivery modality. The fill and finish workflow 2450 of FIG. 37 includes both the LNP (FIG. 35) and the LPX (FIG. 36) workflows and is reflective of the workflows that are possible to execute or perform in connection with the fill and finish modules shown in FIGS. 25 and 28. Stated otherwise, the workflows depicted in FIGS. 35-37 may be carried out in the single fill and finish modules/units depicted in FIGS. 25 and/or 28. The method 2400 may include multiple receiving/handling steps 2402. For example, at step 2404, the method 2400 may include receiving ready-to use (RTU) drug product (i.e., bulk drug product in bags (i.e., rather than in ready-to-use/ready to ship vials), i.e., bDP) from the drug product module or workflow as described herein. At step 2370, the method 2400 may include locking the drug product into the BioNTainer (i.e., into the fill and finish BioNTainer, i.e., into the fill and finish container/module/unit) such that clean room grade C/aseptic conditions are maintained. The method 2400 may generally include preparation of the drug product 2406, followed by filling of the drug product into containers 2412 followed by intermediate storage of uninspected vials 2424 followed by visual inspection 2426, followed by finishing steps 2430. The steps following filling 2412 may be performed in the CNC area 2202 (i.e., controlled not classified area 2202) while the preparation 2406 and filling 2412 steps may be performed within the clean room grade C (CRC) area 2204.

Referring still to FIG. 35, at steps 2408 and 2410, the method 2400 (specifically, the drug product preparation steps 2406) may include sterile filtration, mixing, and cooling of the bulk drug product (bDP) respectively. At steps 2414-2422 and 2242 (that is, the filling 2412 steps) the method 2400 may include material transfer 2414 (for example, of the bulk drug product into the filling machine), filling 2416 of the drug product into containers, stoppering 2418, capping and crimping 2420, tray loading 2422, and locking out of the CRC (clean room grade C) area 2204 to the CNC area 2202. At steps 2432-2438 (i.e., the finishing 2430) the method may include labeling and packaging the filled containers for storage 2432, intermediate storage of the inspected vials 2434, packaging the inspected vials for shipment 2436, and shipping the packaged vials 2438 to one or more CMOs (contract manufacturing organizations), hospitals, clinics, and/or health care providers. In some embodiments, filling 2416 may include bathing the bulk drug product in laminar Grade A, debagging, denesting, and/or delidding in connection with the equipment in the fill and finish modules 90, 360, 370, 380 described herein.

FIG. 36 depicts an overview of an exemplary fill and finish workflow 2440 (or method 2440), according to aspects of the present disclosure. The embodiment of FIG. 36 may be used primarily in connection with drug products that use lipoplexes as the delivery modality (for example, with cancer vaccines). By contrast, the embodiment of FIG. 35 is used primarily with lipid nanoparticle delivery modalities (for example, in connection with infectious disease vaccines). Accordingly, the fill and finish workflow 2240 of FIG. 36 differs from that of FIG. 35 only in that in does not include the drug product preparation steps (2406, 2408, 2410) prior to the fill and finish steps. Referring to FIGS. 34-36, in some embodiments, following locking out to the CNC 2242, the workflow 2360, 2400, 2440 may include visual inspection 2428 (i.e., thereby skipping the intermediate storage step 2424).

FIG. 37 depicts an overview of an exemplary fill and finish workflow 2450 (or method 2450), according to aspects of the present disclosure. The embodiment of FIG. 37 includes various options that are possible using the equipment and configuration of the fill and finish modules described herein (for example, at least in connection with FIGS. 25 and 28). The workflow 2450 generally includes receiving/handling steps 2402, drug product preparation steps 2406, filling steps 2412, and finishing steps such as intermediate storage of vials 2424, 2434, visual inspection 2426, and labeling, packaging, and shipment steps 2432, 2436, 2438, 2474, 2476, 2480, as described herein. In some embodiments, intermediate storage of inspected vials 2434 occurs in the middle of labeling, packaging, and shipment steps (i.e., during the steps included in process steps 2430). In some embodiments, after visual inspection 2428, labeling and packaging 2472 occur, followed by shipment 2478, without any intermediate storage steps. Receiving and handling steps 2402 may include receipt of RTU (ready to use) bulk drug product (bDP) from an external warehouse 2452 (which may be stored 2455) or from an internal source 2456 (for example, internal to the worksite/drug production facility). The RTU drug product may be thawed 2458 and/or homogenized 2460 before being locked into the BioNTainer 2370 (i.e., into the modular fill and finish clean room grade C area 2204). In some embodiments, following homogenization 2460, (for example, in embodiments where the RTU drug product is in the form of a bDP concentrate) a dilution step (not shown) may also be performed.

Referring still to FIG. 37, at step 2462, the method 2450 may include pooling of different single specific bulk drug products (i.e., for use in multivalent embodiments) and/or pooling of the same single specific bulk drug products (i.e., monovalent embodiments). For example, in some embodiments, different drug products are used in connection with a single vaccine or treatment. For example, in some embodiments (for example, when used in connection with cancer vaccines) as many as six (6) different drug substances or drug products, each including different RNA or mRNA lipoplexes, may be included in 6 different drug products, which then need to be pooled into a single drug product prior to fill and finish steps. Accordingly, the fill and finish equipment included in the module/workflow/fill and finish unit 2450 of FIG. 37 includes equipment that enables multiple (for example, six (6)) different drug products to be pooled together prior to fill and finish steps. By way of example, the method 2450 may include mixing five (5) 10 L bags in an mRNA-LNP production run (i.e., for a total volume of 50 L within the mixing unit) or alternatively six (6) 8 L bags in an mRNA lipoplex production run (i.e., for a total volume of 48 L within the mixing unit). In addition, in some embodiments, pooling 2462 includes the pooling of multiple batches of the same drug product prior to fill and finish. At step 2464, the method 2450 may include mixing and/or cooling of the bulk drug product (bDP). At step 2466, the method 2450 may include sterile filtration for processes that are bioburden controlled. At step 2410, the method 2450 may include mixing and/or cooling of the sterile bulk drug product (bDP). As shown in FIG. 37, in some embodiments, pooling and mixing of multiple different types or batches of bulk drug product is not required and homogenized drug product (from step 2460) may proceed directly to the sterile filtration step 2466 after locking-in to the fill and finish BioNTainer (i.e., the fill and finish clean room grade C area 2204). In some embodiments, preparation 2406 of the drug products includes only mixing and cooling of the sterile bulk drug product after locking-in to the clean room area 2204 and before performing the filling steps 2412. In some embodiments, after the mixing and cooling 2464 of bulk drug product that follows the pooling step 2462, the mixed and cooled drug product is ready for filling 2412.

Still referring to FIG. 37, at step 2414, as part of the filling process 2412, the method 2450 may include transferring of drug product material to the filling machine 2414. In some embodiments, transferring of drug product material to the filling machine 2414 includes using one or more filling lines in combination with one or more peristaltic pumps (and or piston pumps) which may be fluidly coupled to the filling line(s) via one or more ports disposed within the filling line(s). At step 2416, the method 2450 may include filling containers (i.e., vials) with drug product followed by stoppering 2418 or semi-stoppering 2468. In embodiments that employ full stoppering at step 2418, the stoppered containers are then capped and crimped (step 2420), loaded onto trays (step 2422) and locked out of the BioNTainer (i.e., the fill and finish grade C clean room (CRC) 2204). In embodiments that employ semi-stoppering at step 2468, the method 2450 may include lyophilization (for example, using the lyophilization equipment 96 shown in FIG. 25) and final stoppering at step 2470, prior to capping and crimping 2420, tray loading 2422 and locking out to the CNC area 2202 (i.e., from the CRC area 2204).

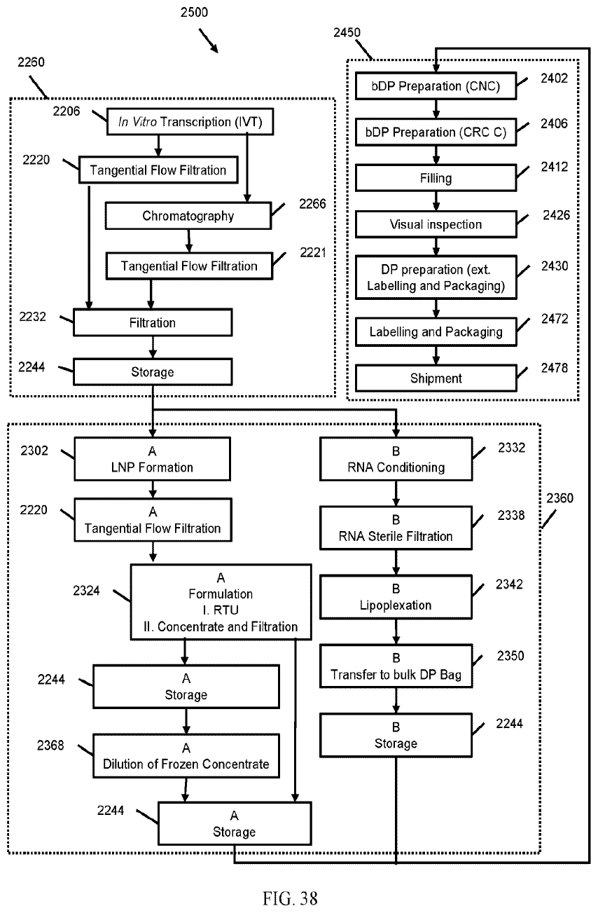

FIG. 38 depicts an overview of an overall, combined workflow 2500, according to aspects of the present disclosure. The workflow generally includes a combined drug substance module/workflow 2260, a combined drug product module/workflow 2360, and a fill and finish module 2450. For example, in connection with the combined drug product module/workflow 2360, the process flow can follow the “A” steps (shown on the left side of box 2360 in FIG. 38) or the “B” steps (right side of box 2360). The “A” steps correspond with LNP formation while the “B” steps correspond with lipoplex formation. In some embodiments, as discussed herein, the “A” steps may be used in connection with the production of infectious disease (e.g., Sars-CoV-2) vaccines while the “B” steps may be used in connection with the production of cancer vaccines. In some embodiments, the drug substance module/workflow 2260 includes optionally performing the filtration step 2232 in connection with LNP drug products. In some embodiments, the drug substance module/workflow 2260 includes performing the chormatography step 2266 (for example, hydrophobic interaction chromatography (HIC)) in connection with lipoplex drug products. The combined drug substance module/workflow 2260 includes options to selectively perform chromatography 2266 and single or multiple TFF steps 2220, 2221 as described herein and as illustrated in FIG. 38. In addition, the combined drug substance module/workflow 2260 includes IVT 2206, filtration 2232 and (optionally) storage 2244. For example, in some embodiments, following filtration 2232, the drug substance/solution is delivered directly to the relevant process flow (i.e., either “A” or “B” shown in the bottom half of FIG. 38) rather than going to storage 2244. The combined drug product module/workflow 2360 includes options for both LNP formation 2302 (including TFF 2220, formulation/concentration/filtration 2324, storage 2244, and/or dilution 2368) and lipoplex formation including RNA conditioning 2332, RNA sterile filtration 2338, lipoplexation 2342, transfer to bulk drug product bags/containers 2350, and/or storage 2244, as described herein and as shown in FIG. 38. The fill and finish module 2450 may include bulk drug product preparation 2402 (in CNC area), further bulk drug product preparation 2406 (in the CRC area) filling 2412, visual inspection 2426, drug product preparation 2430 (i.e., post filling), labelling and packaging 2472, and shipment 2478. In some embodiments, following filling 2412 and visual inspection 2426, drug product preparation 2430 includes transporting the filled vials to a contract manufacturing organization (CMO)/facility where the labelling and packaging 2472 are performed. In certain embodiments, any of the methods and/or workflows presented herein may include additional steps. In certain embodiments, any of the methods and/or workflows presented herein may include doing steps in a different order than what is presented and/or in doing multiple steps at the same time (rather than sequentially). In certain embodiments, any of the methods and/or workflows presented herein may include omitting one or more steps.

According to aspects of the present disclosure, for example, in embodiments where lipoplexes (i.e., lipoplex particles) are produced for the purpose of delivering one or more cancer vaccines or therapies, positively charged liposomes may be mixed with RNA molecules such that the lipids (for example, cationic lipids) and RNA are present at a charge ratio of 1.3 to 2 to facilitate delivery of the resulting lipoplex to a target location. In some embodiments, the lipoplex particles include a phospholipid bilayer structure that encapsulates the RNA molecules. In some embodiments, the lipoplex particles include a cationic lipid, an ionizable aminolipid (for example, including DOPE), and/or a helper lipid. In some embodiments, the lipoplex particle includes a cationic lipid or an ionizable aminolipid in a 2:1 molar ratio with a helper lipid. In some embodiments, the lipoplex particle (i.e., nano particle) comprises a particle size (for example, a Z-average) in a range from about 250 nm to about 700 nm. In some embodiments, the RNA encapsulated and/or contained within the lipoplex particle encodes at least one of a carcinoma antigen, a melanoma-associated antigen, a tyrosinase antigen, and a transmembrane phosphatase with tensin homology (TPTE) antigen. In some embodiments, RNA lipoplex particles that are useful for delivering RNA molecules are described in WO 2023/006920, the entire contents of which are incorporated herein by reference for all purposes.

### Early Warning System

In one or more aspects, the present embodiments are directed to a localized Early Warning System (EWS) for identifying disease Variants of Concern (VOC). In one or more aspects, the present embodiments are directed to systems and method for determining and producing vaccines that target and help to treat local strains of a disease. In some embodiments, Early Warning Systems are used to identify Variants of Concern (i.e., in the context of worldwide spread) based on one or more of: the transmissibility of a strain (i.e., viral fitness or infectivity), the rate of growth of a strain within a human being, the ability of strain to evade existing vaccines, boosters and/or inherent immune responses (i.e., an immune escape score), and the severity of the strain to humans. (See: Early Computational Detection of Potential High Risk SARS-CoV-2 Variants; Beguir et al, (doi: https://doi.org/10.1101/2021.12.24.474095), the disclosure of which is incorporated herein by reference, in its entirety.).

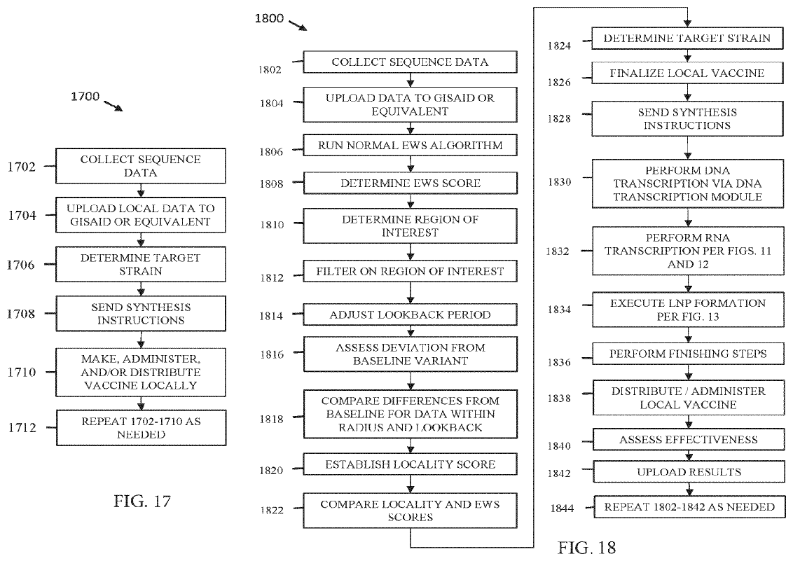

FIG. 17 illustrates a process (or method) 1700 for making vaccines, according to aspects of the present disclosure. At step 1702, the method 1700 may include collecting sequence data (for example, using local sequencing equipment based on test samples taken from a local area). At step 1704, the method 1700 may include uploading the local data to a public database such as GISAID, NCBI, an EWS database, and/or other databases. At step 1706, the method 1700 may include analyzing the data (i.e., data local to the area of interest) to determine a target strain. The target strain may be an actual strain that is prevalent in the area and/or may be a hybrid that combines different segments of sequences of interest to target a hybrid or “synthetic” strain that is representative of one or more local strains. At step 1708, the method 1700 may include sending synthesis instructions from the database and/or cloud-based computing system to a local computing system, the synthesis instructions including sequencing information such that DNA can be created via DNA synthesis. At step 1710, the method 1700 may include making, administering, and/or distributing a vaccine locally, as described herein, the vaccine being based on the synthesis instructions. At step 1712, the method 1700 may include repeating steps 1702-1710 as needed.

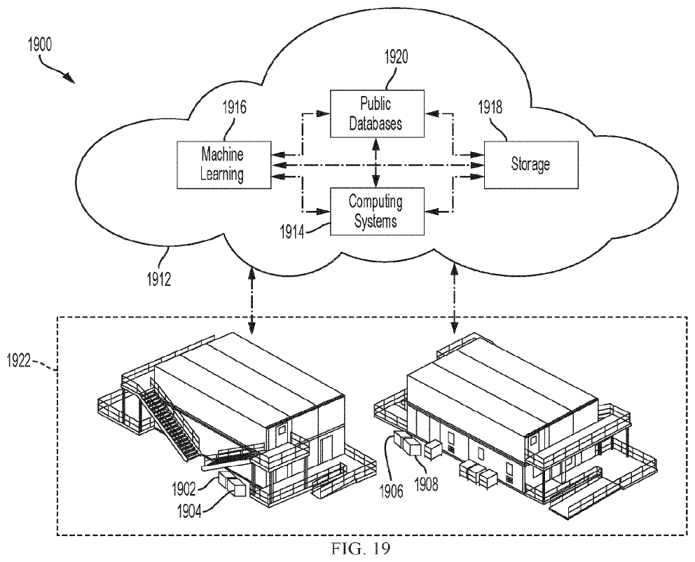

FIG. 18 illustrates a process (or method) 1800 for making vaccines, according to aspects of the present disclosure. At step 1802, the method 1800 may include collecting sequence data. At step 1804, the method 1800 may include uploading the local data to a public database such as GISAID, NCBI, an EWS database, and/or other databases. At step 1806, the method 1800 may include running a conventional EWS algorithm on the collected data to identify and assess potential Variants of Concern based on one or more of the transmissibility of a strain, the rate of growth of a strain within a human, the ability of strain to evade existing vaccines and/or boosters, and the severity of the strain to humans. In some embodiments, running the EWS algorithm includes a lookback period of 1-week (i.e., evaluating data collected in the most recent week). At step 1808, the method 1800 may include determining an EWS score (i.e., quantifying and assigning a score to each evaluated strain in order to determine if a Variant of Concern has been identified for further study, and on which potential future action may be taken). At step 1810, the method 1800 may include determining a region of interest (for example, the local area in which the site is located and/or potential adjacent or proximal areas within which particular outbreak activity has been occurring). In some embodiments, a region of interest may include a simple radius around a geographic area (for example, within 50 miles, 100 miles, 500 miles, 1000 miles, etc. of the site). In some embodiments, a region of interest may include certain population centers that do not fall neatly within a certain radius (since population density is rarely uniform, and transport between regions is also typically non-uniform (and well-traveled routes may be conduits for disease spread)).

Referring still to FIG. 18, at step 1812, the method 1800 may include filtering the data on the public database based on the region of interest (i.e., so as to include only sequence data from strains from within the region of interest). At step 1814, the method 1800 may include adjusting the lookback period (for example, filtering the data based on a particular time period of interest). Adjusting the lookback period may include looking back more than a single week (for example, looking back a month, or several weeks, etc. as may be the case). Adjusting the lookback period may include selecting a range of dates that coincides with the dates that correspond to local outbreaks, etc. At step 1816, the method 1800 may include assessing deviations from the filtered data and one or more baseline variants. Assessing deviations may include creating a deviation between each sequence within the filtered data set and a baseline variant. Assessing deviations may also include creating a deviation between certain subsets of sequences within the filtered data set and a baseline variant. For example, sequences within the filtered data set may be put into one or more bins or subsets, and averaged such that an average or representative sequence may be determined for each subset. Deviations between each representative sequence and the baseline sequence may then be determined. In some embodiments, assessing deviations may include identifying regions of the representative sequence that deviate from the baseline variant. In some embodiments, assessing deviations may also include the specific mutation associated with each nucleotide that deviates from the baseline variant. In some embodiments, the baseline variant may include a previously identified strain that is or was prevalent within the region of interest. In some embodiments, the baseline variant may include a target strain upon which an existing vaccine is based.

Referring still to FIG. 18, at step 1818, the method 1800 may include comparing deviations from the baseline variant for each subset of data within the radius and lookback period (i.e., within the filtered data set) to determine if there are commonalities between the regions of the sequence where deviations occur and/or between the specific mutations for each of the deviations. At step 1820, the method 1800 may include establishing locality scores for each subset within the filtered data subset, the locality scores being based on commonalities that each subset shares with other subsets within the filtered data (thereby identifying the most common strains that are specific to the location and time period of interest). At step 1822, the method 1800 may include comparing locality and EWS scores to assess and/or inform a future direction for Variants of Concern and/or local vaccine decision-making. For example, if the EWS scores are low, it means there likely have not been any Variants of Concern identified from a worldwide outbreak point of view. On the other hand, if the locality score is high, it means that the strains in the region of interest and in the time period of interest vary greatly from a baseline variant, meaning that existing vaccines may not be particularly well-suited for treating local strains within the region of interest, and/or the potential for future mutation is higher since the local strains have already deviated significantly from the baseline variant.

At step 1824, the method 1800 may include determining a target strain based on the EWS score, the locality score, and/or a combination thereof. At step 1826, the method 1800 may include finalizing one or more vaccines (i.e., based on the target strain for the given region of interest, the target strain being an actual strain and/or a hybrid or synthetic strain that prioritizes aspects of sequences resulting from both the EWS and the local strain analyses). At step 1828, the method 1800 may include sending synthesis instructions from the database (or network, or cloud, and/or command center) to the site. At step 1830, the method 1800 may include performing DNA synthesis based on the synthesis instructions using a DNA synthesizer located within the transcription module (and/or within a different module) according to the present disclosure. DNA synthesis may be followed by transcription, according to aspects of the present embodiments, and as described herein. At step 1832, the method 1800 may include preforming RNA transcription per FIGS. 11 and 12. At step 1834, the method 1800 may include performing LNP formation per FIG. 13. At step 1836, the method 1800 may include performing finishing steps (i.e., fill and finish) as described herein. At step 1838, the method 1800 may include distributing and/or administering the local vaccine within the region of interest. At step 1840, the method 1800 may include assessing the effectiveness of the local vaccine for treating the local strain of the disease. At step 1842, the method 1800 may include uploading the effectiveness results to the one or more public databases. At step 1844, the method 1800 may include repeating any of steps 1802-1842 as needed.

FIG. 19 depicts an overview of an exemplary drug product manufacturing enterprise and/or process, according to aspects of the present disclosure. In the embodiment of FIG. 19, certain aspects of the enterprise may be located or occur at the site 1922 or alternatively, on the cloud (or network) 1912. According to aspects of the present embodiments, the site 1922 may include one or more sequencers 1902 to produce sequences of local strain. The site 1922 may also include one or more DNA synthesizers 1906 for synthesizing DNA (upon which vaccines may be generated, according to the present embodiments), the DNA synthesis being based on target strains, as determined from the cloud or network 1912. The cloud or network, which is communicatively coupled to the site 1922 and able to both transmit data to, and receive data from, the site 1922, may include several functions and components including (but not limited to) machine learning 1916, public databases 1920, data storage 1918, and computing systems 1914.

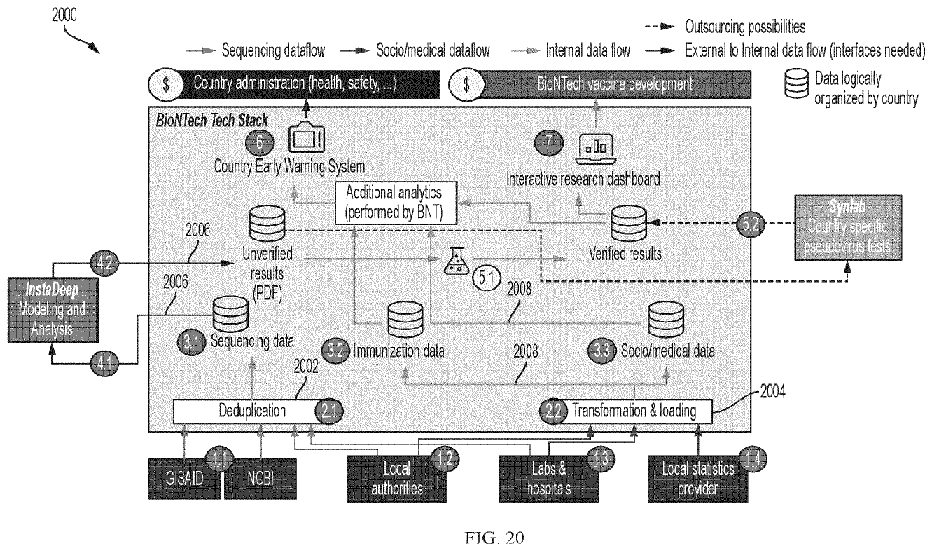

FIG. 20 depicts an overview of another exemplary drug product manufacturing enterprise and/or process 2000, according to aspects of the present disclosure. In the embodiment of FIG. 20, sequencing dataflows (i.e., to deduplication 2002), socio-economic and medical dataflows (i.e., to transformation and loading 2004), external dataflows (i.e., to modelling and analysis), and internal data flows 2008 are illustrated, according to aspects of the present embodiments.

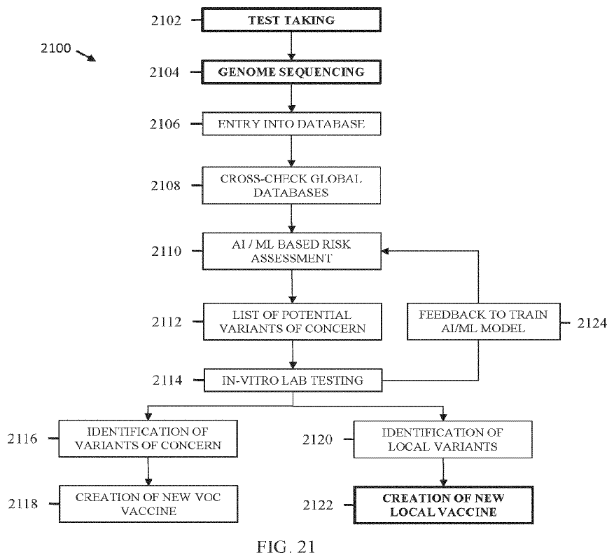

FIG. 21 depicts an overview of another exemplary drug product manufacturing enterprise and/or process (or method) 2100, according to aspects of the present disclosure. At step 2102, the method 2100 may include taking a test (for example, at the site). At step 2104, the method 2100 may include genome sequencing one or more samples from the test. At step 2106, the method 2100 may include entering genome sequencing data into a database. At step 2108, the method 2100 may include cross-checking the entered genome sequencing data against one or more global databases. In some embodiments, cross-checking the entered genome sequencing data against one or more global databases may include comparing the entered genome sequencing data against other local genome sequencing data and/or genome sequencing data from a global sequence repository. If the entered genome sequencing data is not in the repository, it may be added, according to aspects of the present embodiments. At step 2110, the method 2100 may include using machine learning or artificial intelligence to perform one or more risk assessments on the genome sequencing data. At step 2112, the method 2100 may include identifying a list of potential variants of concern based on the one or more AI-based risk assessments. At step 2114, the method 2100 may include performing in-vitro lab testing to verify and confirm the prediction(s) from the AI-based risk assessments. At step 2124, the method 2100 may include providing feedback from the in-vitro lab testing to the AI or machine learning model, in order further refine the AI or machine learning model.

Referring still to FIG. 21, at step 2116, the method 2100 may include identifying one or more variants of concern (VOC) following the confirmation and/or verification during in-vitro lab testing. At step 2118, the method 2100 may include creation of a new VOC vaccine to treat the one or more variants of concern (VOC). At step 2120, the method 2100 may include identifying one or more local variants following the confirmation and/or verification during in-vitro lab testing. At step 2122, the method 2100 may include creation of a new local variant vaccine to treat the one or more local variants. In some embodiments, the bolded steps shown in FIG. 21 (i.e., steps 2102, 2104, and 2122) may occur at the local site while the un-bolded steps (i.e., the remaining steps of FIG. 21) may occur at the enterprise and/or cloud level.

According to the present disclosed embodiments, variant and/or local strain sequencing data from surveillance field testing can be processed in real time (or near real time) without needing to ship samples to centralized testing locations, which could add days to the turnaround time. AI and machine learning running on a supercomputing infrastructure (for example, leveraging a cloud-based network) allows results and analysis to be made available on site. In some embodiments, the disclosed systems and methods rank all known variants for infectivity and immune escape risk potential, allowing a live monitoring of the worldwide pandemic and local outbreaks. Novel variant sequences can be scored and ranked in silico (i.e., via computer modelling) in mere hours (for example, less than 4 hours, less than 3 hours, less than 2 hours, etc.). The present embodiments allow new variants to be identified and added to the global pool or repository more quickly and thoroughly. Similarly, the present disclosure enables the most infectious variants, as well as the most immune escaping variants, to be detected. The present embodiments also provide a ranked list of variants that should be monitored and tested in-vitro, which helps to prioritize how laboratory resources may most effectively be utilized.

The present embodiments provide the ability to produce mRNA drug products (for example, vaccines) at locations worldwide. By incorporating a distributed network of modular drug production installations into a worldwide Early Warning System (EWS), the present embodiments provide infrastructure and on-the-ground capabilities to help battle ongoing and future pandemics. The present disclosure provides systems and methodologies that allow greater access to vaccine and disease treatment, regardless of socio-economic status and other factors. In addition, localized production facilities are well-suited to provide tailored and real-time (or near real-time) response to local outbreaks. The provided ecosystem also increases testing and research capacity and encourages collaboration. The EWS of the present embodiments enables potential identification of pathogens that pose a pandemic threat via AI-powered risk assessment and confirmatory lab testing. In addition, by having a worldwide network of on-the-ground installations, increased transparency of pandemic threats may be realized through faster detection of new variants and/or pathogens. The local modular mRNA vaccine production units of the present embodiments also reduce the dependency of states and countries on external drug supplies and suppliers, thereby ensuring rapid and local responses to potential future pandemic situations.

### EQUIVALENTS

Those skilled in the art will recognize, or be able to ascertain using no more than routine experimentation, many equivalents to the specific embodiments of the invention described herein. Therefore, the scope of the present invention is not intended to be limited to the above Description.

We claim:

1. A drug production facility comprising:
a first module comprising drug substance and drug product manufacturing equipment comprising equipment for producing RNA-based drug substance, lipid nanoparticle (LNP)-based drug products and lipoplex-based drug products; and
a second module comprising fill and finish process equipment.

2. The facility of claim 1, wherein the first module contains:
an RNA-based drug substance; and
at least one of an LNP-based drug product and a lipoplex-based drug product.

3. The facility of claim 1, wherein each of the first module and the second module comprises a Grade C Clean Room area of from about 80 square meters to about 120 square meters.

4. The facility of claim 1, wherein the second module comprises a first machine for debagging, a second machine for denesting, a third machine for filling and a fourth machine for capping and crimping.

5. The facility of claim 4, wherein the second module further comprises at least one conduit supplying laminar Grade A air to an air bathing area, the air bathing area being located in a vicinity of the first machine for debagging is located.

6. The facility of claim 4, wherein the second module further comprises:
a first mixing unit for pooling multiple drug products, each drug product of the multiple drug products comprising different RNA, the multiple drug products being used in a single, multivalent vaccine; and
a second mixing unit for performing sterile filtration.

7. The facility of claim 4, wherein the second module further comprises at least one of: lyophilization equipment, an autocalve, glass washing machinery, sterilizing misting equipment, glove box testing equipment, and a wall pass through.

8. The facility of claim 4, wherein the second module further comprises at least one conduit supplying nitrogen to the filling machine.

9. The facility of claim 7, wherein the second module comprises sterilizing misting equipment, and
wherein the sterilizing misting equipment is configured to provides a sterilizing mist comprising ionized hydrogen peroxide.

10. The facility of claim 4, wherein the first module comprises a chromatograph comprising at least one of a cellulose chromatograph, a chromatograph configured to remove oligo (dT) primers, and a chromatograph configured to perform hydrophobic interaction chromatography (HIC).

11. The facility of claim 4, wherein the second module comprises multiple sensors comprising:
at least one pressure sensor;
at least one combined temperature and humidity sensor
at least one hydrogen peroxid sensor;
at least one ethanol sensor; and
at least one oxygen sensor.

12. The facility of claim 1, wherein the first module contains a lipoplex-based drug product comprising lipoplex particles that comprise a phospholipid bilayer structure that encapsulates RNA molecules of the RNA-based drug substance.

13. The facility of claim 1, wherein the first module contains a lipoplex-based drug product comprising lipoplex particles that comprise a cationic lipid or an ionizable aminolipid in a 2:1 molar ratio with a helper lipid.

14. The facility of claim 1, wherein the first module contains a lipoplex-based drug product comprising lipoplex particles that comprise a particle size in a range from about 250 nm to about 700 nm.

15. The facility of claim 1, wherein the first module contains a lipoplex-based drug product encapsulating the RNA-based drug substance, wherein RNA of the RNA-based drug substance encodes at least one of a carcinoma antigen, a melanoma-associated antigen, a tyrosinase antigen, and a transmembrane phosphatase with tensin homology (TPTE) antigen.

16. A method of selectively producing a filled container containing an RNA-based drug product at a production facility comprising a first module and a second module, the method comprising:
producing an RNA-based drug substance within the first module, wherein producing drug substance comprises:
performing in vitro transcription (IVT);
performing at least one of a tangential flow filtration (TFF) step and a hydrophobic interaction chromatography (HIC) step; and
performing at least one filtration step following TFF and/or HIC;
producing, within the first module, a lipid-based drug product that comprises the RNA-based drug substance, the lipid-based drug product comprising at least one of a lipoplex particle and a lipid nanoparticle (LNP); and
performing a filling process, within the second module, comprising filling at least one fluid container with the lipid-based drug product.

17. The method of claim 16, wherein producing an RNA-based drug substance comprises performing chromatography prior to a TFF step of the at least one tangential flow filtration (TFF) step.

18. The method of claim 16, wherein performing a filling process comprises:
bathing a bag holding the RNA-based drug product in laminar Grade A air while a debagging process occurs to remove the bag; and
filling the container with the RNA-based drug product using an automated filling machine.

19. The method of claim 16, wherein the lipid-based drug product comprises a lipid nanoparticle (LNP), and
wherein producing a lipid-based drug product comprises:
performing an LNP formation step comprising impingement jet mixing;
performing a tangential flow filtration (TFF) step following the LNP formation step; and
performing at least one formulation step following the TFF step.

20. The method of claim 16, wherein the lipid-based drug product comprises a lipoplex particle, and
wherein producing a lipid-based drug product comprises:
conditioning RNA from the RNA-based drug substance;
performing sterile filtration on the conditioned RNA; and
performing a lipoplexation step to produce the lipoplex particle.

21. A portable system for producing a formulation comprising an mRNA-based product, the system comprising:
a first sub-system comprising a drug substance formulation module, the first sub-system comprising:
transcription equipment for forming an RNA solution via in vitro transcription; and
a second sub-system operatively downstream of the first sub-system comprising multiple drug product formation modules, the second sub-system comprising:
an LNP formation module for producing an RNA-LNP preparation from the RNA solution;
a lipoplex formation module for producing an RNA lipoplex preparation from the RNA solution; and
a buffer preparation module,
wherein the portable system is contained within an area no larger than about 75 square meters.

22. The system of claim 21, wherein the lipoplex formation module comprises continuous automated lipoplex formation (CALF) equipment.

23. The system of claim 22, wherein the lipoplex formation module comprises at least one of a mixing unit, a biowelder, a biosealer, a refrigerator, a sonicator, and a bag tester.

24. The system of claim 21, wherein the second sub-system further comprises a shared use area.

25. The system of claim 24, wherein the shared use area comprises at least one of a charging station, a mobile HMI, a magnetic stirrer, and a filter holder.

26. The system of claim 21, wherein the buffer preparation module comprises at least one of a magnetic stirrer, a buffer mixing unit, a pH sensor, and a conductivity sensor.

27. A facility comprising the system of any of the previous claims, the facility further comprising a portable fill and finish module.

28. The facility of claim 27, wherein the fill and finish module comprises lyophilization equipment.

29. The facility of claim 27, wherein the fill and finish module comprises autoclave equipment (e.g., one or more autoclaves).

30. The facility of claim 27, wherein the fill and finish module comprises machine washing equipment.

31. A method for making liposomes in a modular and/or portable production facility, the method comprising:
adjusting a lipid concentraction within a first solution;
adding the lipid concentration to, and mixing it with, ethanol;
sterilizing the mixture via filtration;
injecting the filtered mixture into a spinner flask;
stirring the filtered mixture within the spinner flask;
continuing injection of the mixture into the spinner flask until a desired lipid concentration is reached;
filtering the mixture, thereby forming filtered liposomes; and
diluting the filtered liposomes.

32. The method of claim 31, comprising storing the diluted liposomes in bags.

33. The method of claim 31, wherein filtration comprises filtering with a cellulose acetate filter.

34. The method of claim 31, wherein filtration comprises filtering with a pore size in a range from about 0.1 micrometers to about 0.5 micrometers.

35. The method of claim 31, comprising, prior to adjusting the lipid concentraction within a first solution, providing filtration equipment, injection equipment, and at least one spinner flask to the modular and/or portable production facility.

36. A method for making lipoplexes in a modular and/or portable production facility, the method comprising:
providing an RNA solution and a liposome solution to the modular and/or portable production facility;
adjusting a concentration of RNA in the RNA solution to a concentration of liposomes in the liposome solution;
adding NaCl to the RNA solution for condensation of the RNA; and
mixing the RNA solution and the liposome solution, thereby forming a lipoplex solution.

37. The method of claim 36, wherein mixing the RNA solution and the liposome solution comprises:
transferring both the RNA solution and the liposome solution into large-volume syringes;
mounting both large-volume syringes onto a single syringe pump; and
simultaneously driving the pistons of each of the respective large-volume syringes, thereby enabling lipoplex formation.

38. The method of claim 37, further comprising:
adding a cryoprotectant solution to the lipoplex solution; and
adjusting a concentration of the lipoplex solution to arrive at a final concentration.

39. The method of claim 38, further comprising:
filling containers (for example, glass vials or bottles) with the lipoplex solution;
freezing the filled containers, thereby forming a lipoplex concentrate; and
storing the frozen and filled containers in a temperature-controlled environment.

40. The method of claim 36, wherein adjusting a concentration of RNA in the RNA solution comprises adjusting the RNA concentration in the RNA solution to allow for mixing of identical volumes of RNA and liposomes.

41. The method of claim 36, further comprising providing a pre-sterilized single use fluid path to the modular and/or portable production facility prior to providing the RNA solution and the liposome solution to the modular and/or portable production facility, to allow for safe aseptic handling of the materials (e.g., solutions).

42. The method of claim 36, further comprising providing in-line cleaning systems to the modular and/or portable production facility prior to providing the RNA solution and the liposome solution to the modular and/or portable production facility, to allow for aseptic conditions within at least one multi-use fluid path.

43. The method of claim 36, wherein the single syringe pump comprises a single perfusor pump.

44. The method of claim 36, comprising: providing continuous automated lipoplex formation (CALF) equipment to the modular and/or portable production facility prior to mixing the RNA solution and the liposome solution, wherein mixing the RNA solution and the liposome solution comprises mixing the RNA solution and the liposome solution using the continuous automated lipoplex formation (CALF) equipment.

45. The method of claim 36, wherein mixing the RNA solution and the liposome solution comprises one or more ethanol injection techniques.

46. The method of claim 36, wherein mixing the RNA solution and the liposome solution comprises one or more sonication steps.

47. The method of claim 36, wherein mixing the RNA solution and the liposome solution comprises mixing using at least one of a Y-type mixing element and a T-type mixing element, and
wherein each of the Y-type mixing element and the T-type mixing element comprises an inner diameter in a range from about 1.2 mm and to about 50.0 mm.

48. The method of claim 36, comprising providing at least one of a mixing unit, a biowelder, a biosealer, a refrigerator, a sonicator, and a bag tester to the modular and/or portable production facility prior to mixing the RNA solution and the liposome solution.

49. A portable system for producing a formulation comprising an mRNA-based product, the system comprising:
a first sub-system comprising a drug substance formulation module, the first sub-system comprising:
equipment for forming an RNA solution; and
a second sub-system comprising multiple drug product formation modules, the second sub-system comprising:
an LNP formation module for producing an RNA-LNP preparation from the RNA solution and a lipid solution; and
a lipoplex formation module for producing an RNA-lipoplex preparation from the RNA solution and a liposome solution;
wherein the portable system is configured to selectively produce each of the RNA-LNP preparation and the RNA-lipoplex preparation using the RNA solution.

50. A method for making RNA lipid nanoparticles (LNP) in a modular production facility, the method comprising:
providing an acidified aqueous phase, an RNA solution, and a lipid solution to the modular production facility;
mixing the acidified aqueous phase with the lipid solution, thereby forming a mixture;
performing at least one of filtration, bioburden reduction, freezing, and dilution on the mixture, thereby forming preformed LNPs; and
mixing the RNA solution and the preformed LNPs, thereby forming the RNA lipid nanoparticles.

51. The system of claim 21, comprising at least one in-process monitoring and/or control sensor.

52. The system of claim 51, wherein the at least one in-process monitoring and/or control sensor comprises a sensor for measuring bacteria count and/or a sensor for detecting at least one visible property or characteristic of a liquid.

53. The system of claim 52, comprising a sensor for measuring bacteria count comprising at least one of a spectrophotometer, a dip tester, and an optical sensor configured to measure an optical density within a specific wavelength spectrum.

54. The system of claim 51, comprising a sensor for detecting at least one visible property or characteristic of a liquid,
wherein the at least one property or characteristic comprises a vortex.

55. The system of claim 51, comprising a sensor for detecting at least one visible property or characteristic of a liquid,
wherein the at least one property or characteristic comprises the existence of at least one subvisible particle (SVP).

56. The method of claim 38, wherein the cryoprotectant comprises:
a sucrose buffer in a weight percent range from about 13% to about 18%;
a HEPES buffer at a concentration of about 3.8 mM to about 4.6 mM;
an EDTA butter at a concentration of about 0.8 mM to about 1.6 mM; and
a pH of about 6.3 to about 7.1.

57. A modular drug production facility (or module) comprising a combined process flow capable of producing both lipid nanoparticle (LNPs) and lipoplexes, the facility (or module) comprising:
an LNP formation module;
a lipoplex formation module;
a lipid supply; and
a drug substance/RNA formation module;
wherein each of the LNP formation module and the lipoplex formation module are fluidly coupled downstream of both the lipid supply and the drug substance/RNA formation module.

58. The facility of claim 57, further comprising a buffer exchange/purification module fluidly coupled downstream of both the lipid supply and the drug substance/RNA formation module.

59. An RNA mixture comprising:
from about 42% to about 54% RNA solution by volume;
from about 8% to about 28% elution buffer by volume; and
from about 30% to about 38% dilution buffer by volume.

60. The mixture of claim 59, wherein the elution buffer comprises a chelating agent.

61. The mixture of claim 59, wherein the elution buffer comprises at least one of an 18 mM HEPES buffer at a pH of 7 and an 18 mM ETDA buffer at a pH of 7.

62. The mixture of claim 59, wherein the dilution buffer comprises ammonium sulphate.

63. The mixture of claim 59, wherein the RNA mixture comprises drug substance.

64. A process of making lipid nanoparticles (LNP) comprising impingement jet mixing the RNA solution of claim 59 with an ethanol solution comprising lipids.

65. A bioburden reduction module comprising the RNA mixture of claim 59 disposed within a filter comprising a 0.2 μm pore diameter.

66. The module of claim 65, wherein the RNA mixture comprises mRNA, and wherein the module comprises an mRNA mass to filter surface area ratio in a range from about 1.0 mg/cm\hat{ }2 to about 2.0 mg/cm\hat{ }2.

67. The method of claim 36, wherein the liposome solution comprises:
about 70% by weight phospholipon 100H loaded with sodium hyaluronic acid;
about 20% by weight cholesterol; and
about 10% by weight stearylamine.

68. A mixing unit comprising:
multiple fluid inlets comprising a first fluid inlet and a second fluid inlet, the first fluid inlet comprising a first fluid flowing therethrough, the second fluid inlet comprising a second fluid flowing therethrough,
wherein the second fluid comprises the RNA mixture of claim 39.

69. The mixing unit of the claim 68, wherein the second fluid inlet further comprises an ethanol solution comprising lipids.

70. The system of claim 22, wherein the CALF equipment comprises a low-pulsation peristaltic pump.

71. The system of the claim 70, wherein the low-pulsation peristaltic pump comprises from about 2 to about 4 rollers.

72. The system of claim 70, wherein the low-pulsation peristaltic pump comprises at least one flow rate sensor in a feedback-loop configuration for online-control and real-time adjustment of a fluid flow rate, the fluid comprising at least one of a liposome solution and a lipoplex solution.

73. The system of claim 70, wherein the low-pulsation peristaltic pump comprises two or more channels to reduce pulsations.

74. The system of claim 71, wherein the rollers are angularly offset from one another around an inner circumference of the low-pulsation peristaltic pump such that pulsations are minimized or eliminated.

75. The system of claim 70, wherein the low-pulsation peristaltic pump comprises a dual head,
wherein the low-pulsation peristaltic pump operates at a flow rate in a range from about 1 mL/min to about 500 mL/min, and
wherein the low-pulsation peristaltic pump operates at a delta pressure range (i.e., the pressure difference between a pump discharge and a pump suction) in a range from about 0.05 bar to about 1 bar.

76. The system of claim 22, wherein the CALF equipment comprises a rocker mixer to aid in lipoplexation.

77. The facility of claim 57, further comprising a liposome formation module disposed fluidly downstream of the lipid supply and fluidly upstream of the lipoplex formation module.

78. The facility of claim 77, wherein the drug substance/RNA formation module produces an RNA solution.

79. A method of forming an RNA-lipid solution comprising:
providing the facility of claim 78; and
selectively flowing lipids and RNA solution to the LNP formation module and/or the lipoplex formation module, thereby forming the RNA-lipid solution.

80. The method of claim 79, wherein selectively flowing lipids to the lipoplex formation module comprises:
flowing lipids to the liposome formation module; and
flowing liposomes from the liposome formation module to the lipoplex formation module.

81. The method of claim 36, wherein adjusting a concentration of RNA in the RNA solution comprises mixing positively charged liposomes in the liposome solution with RNA molecules in the RNA solution such that the positively charged lipids and RNA are present at a charge ratio of 1.3 to 2 to facilitate delivery of the resulting lipoplexes in the lipoplex solution to a target location.

82. The method of 36, wherein the liposome solution comprises cationic lipids.

83. The method of 36, wherein the liposome solution comprises at least one of a cationic lipid, an ionizable aminolipid, and a helper lipid.

84. The system of claim 23, comprising a biowelder, wherein the biowelder comprises at least one single use blade and multiple tube holders.
